(19) United States
(12) Patent Application Publication
(10) Pub. No.: US 2023/0323485 A1
(43) Pub. Date: Oct. 12, 2023

(54) METHOD FOR DETERMINING A VIRALLY-INFECTED SUBJECT'S RISK OF DEVELOPING SEVERE SYMPTOMS

(71) Applicant: The Board of Trustees of the Leland Stanford Junior University, Stanford, CA (US)

(72) Inventors: Purvesh KHATRI, Menlo Park, CA (US); Aditya Manohar RAO, Stanford, CA (US); Michele DONATO, Stanford, CA (US); Denis Dermadi BEBEK, San Francisco, CA (US); Hong ZHENG, Stanford, CA (US); Lara JONES, Stanford, CA (US); Jia Ying TOH, Stanford, CA (US)

(21) Appl. No.: 18/026,823

(22) PCT Filed: Sep. 23, 2021
(86) PCT No.: PCT/US2021/051830

§ 371 Date: Mar. 16, 2023

Related U.S. Application Data
(60) Provisional application No. 63/083,692, filed on Sep. 25, 2020.

(51) Int. Cl. C12Q 1/70; A61K 45/06

(52) U.S. Cl. CPC C12Q 1/701; A61K 45/06; C12Q 2600/158

(57) ABSTRACT

This disclosure provides a gene expression-based method for determining a virally-infected subject's risk of developing severe symptoms. In some embodiments, the method may comprise measuring the amount of RNA transcripts encoded by at least two genes in a sample of RNA obtained from the subject, to obtain gene expression data; and based on the gene expression data, providing a report indicating the subject's risk of developing severe symptoms. Kits and methods of treatment are also provided.

### CROSS-REFERENCING

This application claims the benefit of U.S. provisional application Ser. No. 63/083,692, filed on Sep. 25, 2020, which application is incorporated by reference herein.

### GOVERNMENT RIGHTS

This invention was made with Government support under contract AI109662 awarded by the National Institutes of Health. The Government has certain rights in the invention.

### BACKGROUND

Outbreaks of infectious diseases globally have been increasing steadily over the last 40 years (Christiansen, 2018). The first two decades of the 21st century have been marked by seven outbreaks of novel viral infections that include severe acute respiratory syndrome coronavirus (SARS-CoV-1; 2002), H1 N1 influenza (2009), Middle East Respiratory Syndrome Coronavirus (MERS-CoV; 2012), chikungunya (2014), Ebola (2014), Zika (2015), and severe acute respiratory syndrome coronavirus 2 (SARS-CoV-2). Five of these outbreaks were in the last decade, of which four resulted in pandemics (David M Morens and Anthony S Fauci, 2020). In each viral outbreak, there is an urgent need for diagnostic and prognostic tests for accurately diagnosing patients at high risk of severe outcome who should be admitted to hospitals, and those with mild infection who can recover at home. The ongoing SARS-CoV-2 pandemic, where approximately 80% of infected patients have mild infection, and 20% have severe illness requiring hospitalization and critical care (Wu and McGoogan, 2020), has acutely demonstrated the need for such a test to reduce the risk of hospital overrun, shortage of supplies, and the resulting socioeconomic costs.

The current armamentarium for identifying high-risk patients is comprised of lab tests (e.g., white blood cell count differentials, Procalcitonin, interleukin-6 and -8, lactate dehydrogenase, C-reactive protein) and standardized severity of illness scores designed for predicting mortality among the critically ill (e.g., PRISM, SOFA, APACHE). In a triage setting during viral outbreaks, these have limited clinical utility as they are non-specific markers of inflammation and late predictors of mortality (Falcão et al., 2019; Liu et al., 2020; Rast et al., 2014).

### SUMMARY

Based on transcriptomic data, a virally-infected subject's risk of developing severe symptoms can be determined. Put another way, the method provides a way to determine the risk of a patient of developing severe symptoms, where the patient is infected by a virus.

In some embodiments, the method may comprise:
(a) measuring the amount of RNA transcripts encoded by at least two of HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFITM1, DDB1, BCL2L11, LAPTM4A, KIF23, TYK2, PIK3R1, BANF1, TRIM28, SOCS6, LRBA, ANXA2, IFITM3, CREG1, and NAPA in a sample of RNA obtained from the subject, to obtain gene expression data; and
(b) based on the gene expression data, providing a report indicating the subject's risk of developing severe symptoms.

In these embodiments: (i) increased expression of BCL6, NQO2, ORM1, DEFA4, CTSG, LCN2, AZU1, TXN, CCL2, CEACAM8, AQP9, GRN, CAMP, TLR2, ANXA3, SLPI, KLHL2, CEP55, SRGN, TRIP13, PRC1, TCEAL9, BCAT1, FURIN, ACSL1, HMMR, UBE2L6, CASP7, OLR1, SCAND1, DOK3, KIF15, ATP8B4, IGFBP2, IFITM2, PFKFB4, VAMP5, ELL2, POMP, H1-0, ADM, VRK2, MAFB, CDT1, OASL, TMEM123, TLN1, NUCB1, FAM8A1, BTBD7, ATG3, BCL2A1, IFITM1, BCL2L11, KIF23, SOCS6, ANXA2, IFITM3, CREG1 and NAPA; and (ii) decreased expression of HLA-DPB1, KLRB1, DOK2, KLRG1, KLRD1, EPHX2, EXOC2, PRF1, PRSS23, TRIB2, EZH1, BUB3, ITGB7, SIDT1, RAD23B, ARHGAP45, MAP3K4, USP11, SMYD2, SSR2, IL7R, FBLN5, TRAF5, TRAF31P3, CCR7, LTBP3, CHMP7, PITPNC1, RBM15B, DDB1, LAPTM4A, TYK2, PIK3R1, BANF1, TRIM28 and LRBA increases the risk of the subject will have severe symptoms.

In some embodiments, the method may be for treating a subject having a viral infection. In these embodiments, the method may comprise:
(a) receiving a report indicating the subject's risk of developing severe symptoms, wherein the report is based on the gene expression data obtained by measuring the amount of RNA transcripts encoded by at least two of HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFfTM1, DDB1, BCL2L11, LAPTM4A, K1F23, TYK2, PIK3R1, BANF1, TRIM28, SOCS6, LRBA, ANXA2, IFITM3, CREG1, and NAPA in a sample of RNA obtained from the subject; and
(b) treating the subject based on whether the subject has a high risk of developing severe symptoms.

In these embodiments: (i) increased expression of BCL6, NQO2, ORM1, DEFA4, CTSG, LCN2, AZU1, TXN, CCL2, CEACAM8, AQP9, GRN, CAMP, TLR2, ANXA3, SLPI, KLHL2, CEP55, SRGN, TRIP13, PRC1, TCEAL9, BCAT1, FURIN, ACSL1, HMMR, UBE2L6, CASP7, OLR1, SCAND1, DOK3, KIF15, ATP8B4, IGFBP2, IFITM2, PFKFB4, VAMP5, ELL2, POMP, H1-0, ADM, VRK2, MAFB, CDT1, OASL, TMEM123, TLN1, NUCB1, FAM8A1, BTBD7, ATG3, BCL2A1, IFfTM1, BCL2L11, K1F23, SOCS6, ANXA2, IFITM3, CREG1 and NAPA; and (ii) decreased expression of HLA-DPB1, KLRB1, DOK2, KLRG1, KLRD1, EPHX2, EXOC2, PRF1, PRSS23, TRIB2, EZH1, BUB3, ITGB7, SIDT1, RAD23B, ARHGAP45, MAP3K4, USP11, SMYD2, SSR2, IL7R, FBLN5, TRAF5, TRAF31P3, CCR7, LTBP3, CHMP7, PITPNC1, RBM15B, DDB1, LAPTM4A, TYK2, PIK3R1, BANF1, TRIM28 and LRBA increases the risk of the subject will have severe symptoms.

In some embodiments, the method may comprise the risk to a threshold, determining that the risk is above a threshold, and administering intensive care or an antiviral therapy to the patient.

Kits for performing the method are also provided.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1: Conserved host response to viral infections, represented by the MVS, is associated with severity. a) Overview of datasets used for analysis (left), and criteria for assigning viral infection severity categories to samples (right). The 4,780 bulk transcriptome samples from 26 datasets were divided into discovery and validation cohorts. The “No symptoms” category includes both individuals with asymptomatic viral infection or convalescents. b) ROC curves for distinguishing patients with viral infection of varying severity from healthy controls using the MVS score in 1,674 samples across 19 datasets. The AUROC increased with severity of viral infection. c) Distribution of the MVS scores across the severity of viral infection in 1,674 samples in 19 datasets. Each point in the violin plot represents a blood sample. Jonckheere-Terpstra (JT) trend test was used to assess the significance of the trend of the MVS score over severity of infection. P-values for the comparison of MVS scores in two groups were computed using Mann-Whitney U test.d) Validation of correlation between the MVS score severity of viral infection in 4 independent RNA-seq datasets from patients with SARS-CoV-2, chikungunya, or Ebola infection. None of these viruses were used in the discovery, demonstrating generalizability of the MVS to previously unseen viruses. e) Positive correlation between the MVS score and the number of viral reads detected in blood samples in 3 independent RNA-seq datasets. Each point represents a sample. The X axis represents the number of viral reads, and the Y axis represents the MVS score for each sample.

FIG. 2: Single-cell RNA-seq identifies monocytes as the primary source of the MVS. a-d) The MVS score was higher in myeloid cells from hospitalized patients with viral infection (i.e., moderate, serious, critical, and fatal). UMAP visualization of 264,224 immune cells from 71 PBMC samples in three independent scRNA-seq datasets colored by a) cohort, b) cell type, c) MVS score, and d) severity of viral infection. e) Circle map depicting the average MVS score in each cell type in each category of viral infection severity. The color of the circle represents the average MVS score in each cell type. The size of the circle describes the variability of MVS score in the cell type, with larger size representing lower variability. The barplot on the right of the circle map shows the mean proportion of each cell type in each severity category. A detailed sample-level MVS score distribution across cell types can be found in Figure S2A. f-h) Proportions of myeloid cells and the MVS score at a single cell level increase with severity of viral infection predominantly driven by CD14+ monocytes. f) MVS score in each cell in myeloid cells, CD14+ monocytes, and CD16+ monocytes, g) Proportion of myeloid cells, CD14+ monocytes, and CD16+ monocytes in each individual, and h) correlation between mean the MVS score and cellular proportion in each individual. i-k) In silico deconvolution of bulk transcriptome profiles from healthy controls and patients with viral infection validates increased CD14+ monocytes and reduced CD16+ monocytes with increased severity in 2,027 blood samples from 32 independent cohorts. Forest plots for change in proportions of monocytes, CD14+ monocytes, and CD16+ monocytes in i) non-severe patients compared to healthy controls, j) severe patients compared to healthy controls, and k) severe patients compared to non-severe patients. The x axes represent standardized mean difference between two groups, computed as Hedges' g, in log 2 scale. The size of the rectangles is proportional to the standard error of mean difference in the study. Whiskers represent the 95% confidence interval. The diamonds represent overall, combined mean difference for a given cell type in a given comparison. Width of the diamonds represents the 95% confidence interval of overall mean difference.

FIG. 3: The MVS identifies distinct clusters of patients with non-severe and severe viral infections. a-c) UMAP visualizations of 1,674 samples in 19 datasets colored by a) virus b) MVS score, and c) severity of viral infections show distinct clusters of healthy controls and patients with non-severe and severe viral infections. d-e) Projection of independent cohorts on the UMAP space obtained from the discovery cohort: d) 2,518 samples from seven challenge studies using influenza, RSV, or HRV in GSE73072 and e) 86 samples from the SARS-CoV-2 cohort.

FIG. 4: Patients with non-severe and severe viral infections follow divergent disease trajectories. a) Trajectory analysis of 3,183 samples from 25 cohorts using dSpace. The first two principal components of dSpace distinguish the samples by severity category. b) Clustering of samples using dSpace. c) Proportion of samples for each severity category in each cluster. Clusters 1-5 were predominantly composed of healthy controls and patients with asymptomatic viral infection or convalescents, clusters 6-10 and 13-20 were predominantly composed of patients with non-severe and severe viral infection. d) A principal line on dSpace coordinates identified by trajectory analysis. The red and purple colors of the line ends indicate the severe and non-severe trajectories, respectively.

FIG. 5: Immune responses from NK cells, myeloid cell-derived suppression, and hematopoiesis are associated with severity of viral infection. Violin plots: each dot represents a sample, and the Y-axis represents expression of the corresponding gene in a sample. Box plots: each dot represents a sample, and the Y-axis represents proportion of the corresponding cell type in a sample. Forest plots: represent comparison of change in proportions between two groups for a given immune cell type, obtained by in silico cellular deconvolution of blood samples, where the X-axis represents standardized mean difference between two groups, computed as Hedges' g, in log 2 scale. The size of the rectangles is proportional to the standard error of mean difference in the study. Whiskers represent the 95% confidence interval. The diamonds represent overall, combined mean difference for a given cell type in a given comparison. Width of the diamonds represents the 95% confidence interval of overall mean difference. a) Gene expression heatmap of the 96 severity trajectory-defining genes. Rows represent genes and columns represent samples, ordered by position along the disease trajectory. The dendrogram represents hierarchical clustering performed on the rows of the heatmap. Colors of the dendrograms indicate clusters of genes based on the relationship between each gene expression profile and severity of infection. b) Effect size of each gene, computed as Hedge's g, in a given cell type compared to all other cell types and correlation of each gene with severity of viral infection. c-d) Expression of NK cell-specific genes is negatively correlated with the severity of viral infection in c) 3,183 samples across 25 cohorts used for discovery and d) an independent cohort of 86 samples from healthy controls and patients with SARS-CoV-2 infection used for validation. e) Proportions of NK cells were significantly lower in patients with severe viral infections compared to healthy controls (top panel) and non-severe viral infections (bottom panel). f) Proportions of NK cells reduce with severity of viral infection in three independent scRNA-seq cohorts. g-i) Proportions of MDSCs are higher in patients with severe viral infection. g-h) Expression of CEACAM8, a marker of PMN-derived MDSCs, and IL4R, a marker of monocyte-derived MDSC, increases with severity of viral infection in g) 3,183 samples across 25 cohorts used for discovery and h) an independent cohort of 86 samples from healthy controls and patients with SARS-CoV-2 infection used for validation. i) In silico cellular deconvolution analysis showed proportions of pro-inflammatory macrophages (M1) increase during viral infection irrespective of severity, but proportions of anti-inflammatory macrophages (M2) decrease in patients with non-severe viral infection, and increase in patients with severe viral infection. j-k) Expression of HSPC-specific genes is positively correlated with the severity of viral infection in j) 3,183 samples across 25 cohorts used for discovery and k) an independent cohort of 86 samples from healthy controls and patients with SARS-CoV-2 infection used for validation. 1) Proportions of HSPCs were significantly higher in patients with severe viral infections compared to healthy controls (top panel) and non-severe viral infections (bottom panel). m) Proportions of HSPCs increase with severity of viral infection in three independent scRNA-seq cohorts. n-o) Genes expressed at higher level in patients with mild or moderate viral infection compared to healthy controls and those with severe viral infection in n) 3,183 samples across 25 cohorts used for discovery and o) an independent cohort of 86 samples from healthy controls and patients with SARS-CoV-2 infection used for validation.

FIG. 6: Coordinated protective and deleterious host response modules associated with severity of viral infection. a) The module score for 3,183 samples from 25 cohorts in the four modules. The module score of a sample is defined as the geometric mean of expression of genes in each module in the sample. b-c) Pairwise Spearman's rank correlation coefficient between genes in each module in healthy controls and patients with mild or severe viral infection. b) The width and color of a line connecting two genes represents a correlation value between two genes. The width of the line indicates strength of correlation; red and blue color indicate positive and negative correlation, respectively. c) Genes in modules 1, 2 and 4 are more correlated with each other in severe than mild infections, whereas genes in module 3 are more correlated with each other in mild than severe infections. Each dot in the violin plots represents the correlation between a pair of genes. P-values for the comparisons in the violin plots were computed using Wilcoxon signed-rank test.

FIG. 7: Host response modules improve classification of patients with severe and non-severe viral infections. a) Each of the four module scores across the 3,183 dSpace samples. b) The SoM score is calculated by taking the sum of the module 1 and 2 scores divided by the sum of the module 3 and 4 scores. c-d) The SoM score distinguishes mild and severe viral infections in the c) discovery cohort and d) validation cohort. Each point in the violin plots represents a sample. P-values for the comparisons of SoM scores between groups were computed using Mann-Whitney U test.

FIG. 8: MVS score is associated with severity of viral infection in each dataset in discovery and validation cohorts. a) ROC curves for the MVS score in each dataset in the discovery cohorts. AUROC values varied from 0.859 (95% Cl 0.69-1) to 1 (95% CI 1-1). b) ROC curves for the MVS score in 4 independent cohorts profiled using RNAseq. AUROC values varied from 0.84 (95% Cl 0.76-0.92) to 0.972 (95% Cl 0.932-1). c) Violin plots of the MVS score for all samples across 19 datasets.

FIG. 9: MVS score is predominantly from the myeloid cells and Neutrophil proportion is higher in patients with severe viral infection. a) MVS score for cell types in each sample of the three scRNA-seq cohorts. Red color indicates high score and blue indicates low score. The size of the circle is proportional to the variability of the MVS score across all cells of a specific cell type in a sample. The bar plot on the right of this panel shows the cell type proportions of each sample. b) Forest plots for the comparisons between proportions of neutrophils in bulk transcriptomic profiles. Each row represents an effect size of the comparison of non-severe patients vs healthy controls (top), severe patients vs healthy controls (center), and severe vs non-severe patients (bottom).

FIG. 10: Samples from viral challenge studies almost exclusively cluster with samples from non-severe viral infection within dSpace. a) dSpace trajectory analysis of 3,183 samples from 25 cohorts, including 1,509 samples from 4 viral challenge studies (2 influenza, 1 HRV, 1 RSV). Each point represents a sample; viral challenge study samples are demarcated as triangles. b) Proportion of samples for each severity category or challenge study group within each cluster.

FIG. 11: Expression levels of genes associated with NK cells, myeloid cell-derived suppression, HSPCs, and an overall protective host response correlate with severity of viral infection. a-b) KLRD1 and PIK3R1 expression levels in a) the discovery cohort and b) SARS-CoV-2 infection. c-d) Expression levels of myeloid cell-associated genes, including MDSC markers and ORM1 in c) the discovery cohort and d) SARS-CoV-2 infection. e-f) Expression levels of genes over-expressed in patients with severe viral infection but not in those with non-severe viral infections compared to healthy controls, and preferentially expressed in circulating HSPCs in e) the discovery cohort and f) SARS-CoV-2 infection. g-h) Expression levels of genes identified to be significantly higher in patients with mild viral infection compared to those with serious, critical, or fatal viral infection, or healthy controls, in g) the discovery cohort and h) SARS-CoV-2 infection.

FIG. 12: The interferon-induced genes (IFITM1, IFITM2, IFITM3) and type I and II interferon receptors are highly correlated with protective response genes in mild but not severe viral infection. a) Expression of IFITM1, IFITM2, IFITM3, and type I and II interferon receptors across severity categories in patients with different viral infections including SARS-CoV-2. Each point in the violin plots represents a sample. b) Boxplots representing the correlation between IFITMs and type I and II interferon receptors, and the genes belonging to the protective response module. Each point represents a correlation between a gene pair, and lines connect the same pair across severity categories. P-values for the comparison between severity categories were computed using Wilcoxon signed-rank test. c) Correlation between protective response genes and interferon receptor genes.

FIG. 13: SoM score distinguishes mild and severe viral infection in discovery and validation cohorts with higher accuracy than the MVS score. a-b) The ROC curves of the MVS score for differentiating between severity categories of viral infection in a) discovery and b) validation cohorts. c-d) The ROC curves of the SoM score for differentiating between severity categories of viral infection in c) discovery and d) validation cohorts.

FIG. 14: shows the SoM score from nasal swab samples correlates with severity of viral infection (A), and differentiate ICU patients from outpatients (B).

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that, as used in this specification and the appended claims, the singular forms “a”, “an” and “the” include plural referents unless the content clearly dictates otherwise. Thus, for example, reference to “an agonist” includes a mixture of two or more such agonists, and the like.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Diagnostic Methods

As noted above, a method for determining a virally-infected subject's risk of developing of severe symptoms is provided. In these embodiments, the method may comprise: (a) measuring the amount of RNA transcripts encoded by at least two (e.g., at least 3, at least 4, at least 5, at least 10, at least 20, at least 30, at least 40 or all of) of HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFfTM1, DDB1, BCL2L11, LAPTM4A, K1F23, TYK2, PIK3R1, BANF1, TRIM28, SOCS6, LRBA, ANXA2, IFITM3, CREG1, and NAPA in a sample of RNA obtained from the subject, to obtain gene expression data; and (b) based on the gene expression data, providing a report indicating the risk of the subject developing severe symptoms.

As noted above, in these embodiments: (i) increased expression of BCL6, NQO2, ORM1, DEFA4, CTSG, LCN2, AZU1, TXN, CCL2, CEACAM8, AQP9, GRN, CAMP, TLR2, ANXA3, SLPI, KLHL2, CEP55, SRGN, TRIP13, PRC1, TCEAL9, BCAT1, FURIN, ACSL1, HMMR, UBE2L6, CASP7, OLR1, SCAND1, DOK3, K1F15, ATP8B4, IGFBP2, IFITM2, PFKFB4, VAMP5, ELL2, POMP, H1-0, ADM, VRK2, MAFB, CDT1, OASL, TMEM123, TLN1, NUCB1, FAM8A1, BTBD7, ATG3, BCL2A1, IFfTM1, BCL2L11, K1F23, SOCS6, ANXA2, IFfTM3, CREG1 and NAPA; and (ii) decreased expression of HLA-DPB1, KLRB1, DOK2, KLRG1, KLRD1, EPHX2, EXOC2, PRF1, PRSS23, TRIB2, EZH1, BUB3, ITGB7, SIDT1, RAD23B, ARHGAP45, MAP3K4, USP11, SMYD2, SSR2, IL7R, FBLN5, TRAF5, TRAF31P3, CCR7, LTBP3, CHMP7, PITPNC1, RBM15B, DDB1, LAPTM4A, TYK2, PIK3R1, BANF1, TRIM28 and LRBA increases the risk of the subject will have severe symptoms.

The following table shows the individual AUROC for individual genes listed above. In the table, the first column is the gene name. Second column is AUROC value as a measure of distinguishing whether a patient with viral infection will have a non-severe or severe outcome using a single gene. Third column is AUROC value as a measure of predicting whether a hospitalized patient with viral infection will have a severe outcome or not. Genes with positive correlation to severe outcomes annotated as being “up” and genes with negative correlation to severe outcomes are annotated as being “down”. As is known, the AUROC indicates how capable a model is of distinguishing between classes. The higher the AUROC, the better the model is at predicting different categories of subject. For example, the higher the AUROC (i.e., the closer the score is to 1) the better the model is at distinguishing between different types of patients.

| | Severe vs. | Severe vs. | Correlation | |
|---|---|---|---|---|
| | Non-Severe | Moderate | with | |
| Gene | (AUROC) | (AUROC) | severity | Up/Down |
| HLA-DPB1 | 0.8127 | 0.7307 | −0.41 | Down |
| BCL6 | 0.7963 | 0.7241 | 0.35 | Up |
| NQO2 | 0.7980 | 0.7030 | 0.23 | Up |
| ORM1 | 0.7837 | 0.6810 | 0.16 | Up |
| DEFA4 | 0.7798 | 0.6623 | 0.29 | Up |
| KLRB1 | 0.7735 | 0.6681 | −0.49 | Down |
| CTSG | 0.7749 | 0.6448 | 0.28 | Up |
| LCN2 | 0.7680 | 0.6341 | 0.35 | Up |
| AZU1 | 0.7684 | 0.6320 | 0.27 | Up |
| TXN | 0.7557 | 0.6291 | 0.49 | Up |
| DOK2 | 0.6807 | 0.6866 | −0.26 | Down |
| CCL2 | 0.7375 | 0.6272 | 0.26 | Up |
| CEACAM8 | 0.7426 | 0.6173 | 0.31 | Up |
| AQP9 | 0.7268 | 0.6264 | 0.41 | Up |
| KLRG1 | 0.6801 | 0.6643 | −0.38 | Down |
| KLRD1 | 0.6630 | 0.6813 | −0.27 | Down |
| EPHX2 | 0.6746 | 0.6647 | −0.46 | Down |
| GRN | 0.7210 | 0.6118 | 0.51 | Up |
| CAMP | 0.7640 | 0.5652 | 0.4 | Up |
| TLR2 | 0.7168 | 0.6049 | 0.25 | Up |
| ANXA3 | 0.6804 | 0.6403 | 0.52 | Up |
| SLPI | 0.7093 | 0.6108 | 0.36 | Up |
| KLHL2 | 0.6867 | 0.6330 | 0.2 | Up |
| CEP55 | 0.7396 | 0.5724 | 0.45 | Up |
| SRGN | 0.6467 | 0.6540 | 0.38 | Up |
| TRIP13 | 0.7216 | 0.5713 | 0.39 | Up |
| PRC1 | 0.7250 | 0.5643 | 0.37 | Up |
| TCEAL9 | 0.6769 | 0.6092 | 0.35 | Up |
| EXOC2 | 0.6720 | 0.6127 | −0.22 | Down |
| BCAT1 | 0.6880 | 0.5934 | 0.41 | Up |
| PRF1 | 0.6152 | 0.6625 | −0.24 | Down |
| PRSS23 | 0.6537 | 0.6195 | −0.33 | Down |
| TRIB2 | 0.6574 | 0.6155 | −0.29 | Down |
| FURIN | 0.6804 | 0.5911 | 0.17 | Up |
| ACSL1 | 0.6689 | 0.6014 | 0.38 | Up |
| EZH1 | 0.6849 | 0.5773 | −0.29 | Down |
| HMMR | 0.7058 | 0.5558 | 0.46 | Up |
| UBE2L6 | 0.6887 | 0.5713 | 0.29 | Up |
| CASP7 | 0.6720 | 0.5848 | 0.09 | Up |
| OLR1 | 0.6570 | 0.5995 | 0.33 | Up |
| BUB3 | 0.6500 | 0.6057 | −0.4 | Down |
| SCAND1 | 0.6870 | 0.5687 | 0.2 | Up |
| ITGB7 | 0.6214 | 0.6304 | −0.25 | Down |
| DOK3 | 0.6877 | 0.5626 | 0.44 | Up |
| SIDT1 | 0.6462 | 0.6017 | −0.44 | Down |
| RAD23B | 0.6536 | 0.5924 | −0.04 | Down |
| KIF15 | 0.6993 | 0.5441 | 0.43 | Up |
| ARHGAP45 | 0.5833 | 0.6588 | −0.38 | Down |
| MAP3K4 | 0.6019 | 0.6401 | −0.42 | Down |
| ATP8B4 | 0.6452 | 0.5915 | 0.48 | Up |
| IGFBP2 | 0.6407 | 0.5915 | 0.26 | Up |
| IFITM2 | 0.7087 | 0.5228 | 0.49 | Up |
| USP11 | 0.6014 | 0.6271 | −0.4 | Down |
| SMYD2 | 0.6375 | 0.5852 | −0.39 | Down |
| PFKFB4 | 0.6572 | 0.5631 | 0.43 | Up |
| VAMP5 | 0.6381 | 0.5785 | 0.46 | Up |
| ELL2 | 0.6402 | 0.5720 | 0.41 | Up |
| POMP | 0.6016 | 0.6099 | 0.31 | Up |
| H1-0 | 0.6461 | 0.5599 | 0.45 | Up |
| ADM | 0.6268 | 0.5771 | 0.56 | Up |
| SSR2 | 0.5890 | 0.6063 | −0.34 | Down |
| VRK2 | 0.6372 | 0.5576 | 0.25 | Up |
| IL7R | 0.5946 | 0.5957 | −0.4 | Down |
| FBLN5 | 0.6042 | 0.5855 | −0.3 | Down |
| MAFB | 0.6083 | 0.5736 | 0.32 | Up |
| TRAF5 | 0.5785 | 0.5999 | −0.54 | Down |
| CDT1 | 0.6703 | 0.5072 | 0.42 | Up |
| OASL | 0.6147 | 0.5563 | 0.54 | Up |
| TRAF3IP3 | 0.5544 | 0.6153 | −0.38 | Down |
| TMEM123 | 0.6537 | 0.5128 | 0.16 | Up |
| TLN1 | 0.6205 | 0.5353 | 0.16 | Up |
| CCR7 | 0.5830 | 0.5708 | −0.4 | Down |
| LTBP3 | 0.5681 | 0.5786 | −0.43 | Down |
| CHMP7 | 0.5747 | 0.5705 | −0.47 | Down |
| PITPNC1 | 0.5489 | 0.5954 | −0.51 | Down |
| NUCB1 | 0.5625 | 0.5774 | 0.38 | Up |
| RBM15B | 0.5717 | 0.5610 | −0.37 | Down |
| FAM8A1 | 0.5901 | 0.5314 | 0.08 | Up |
| BTBD7 | 0.5472 | 0.5641 | 0.22 | Up |
| ATG3 | 0.5761 | 0.5260 | 0.18 | Up |
| BCL2A1 | 0.5527 | 0.5454 | 0.33 | Up |
| IFITM1 | 0.5851 | 0.5099 | 0.54 | Up |
| DDB1 | 0.5213 | 0.5721 | −0.41 | Down |
| BCL2L11 | 0.5903 | 0.4960 | 0.39 | Up |
| LAPTM4A | 0.5902 | 0.4882 | −0.12 | Down |
| KIF23 | 0.5830 | 0.4890 | 0.47 | Up |
| TYK2 | 0.6407 | 0.4275 | −0.02 | Down |
| PIK3R1 | 0.5642 | 0.4942 | −0.33 | Down |
| BANF1 | 0.5210 | 0.5328 | −0.21 | Down |
| TRIM28 | 0.4973 | 0.5488 | −0.36 | Down |
| SOCS6 | 0.5015 | 0.5436 | 0.23 | Up |
| LRBA | 0.5276 | 0.5014 | −0.41 | Down |
| ANXA2 | 0.4742 | 0.5518 | 0.13 | Up |
| IFITM3 | 0.4907 | 0.5325 | 0.59 | Up |
| CREG1 | 0.5191 | 0.4854 | 0.33 | Up |
| NAPA | 0.4415 | 0.5408 | 0.16 | Up |

The method can be practiced with a number of different gene combinations. In some embodiments, the RNA transcripts analyzed may include the transcripts of the top two, top 3, top 4, top 5, top 6 or top 7 of the genes from table shown above.

In some embodiments, the RNA transcripts analyzed may include the transcripts of any of the gene combinations shown below, although several combinations of genes that have an AUROC value of at least 0.8 could be used. For example, the assay may use any of the following combinations of genes: HLA-DPB1 and BCL6, HLA-DPB1 and NQO2, HLA-DPB1 and ORM1, HLA-DPB1 and DEFA4, HLA-DPB1 and KLRB1, HLA-DPB1 and CTSG, HLA-DPB1 and LCN2, HLA-DPB1 and AZU1, HLA-DPB1 and TXN, BCL6 and HLA-DPB1, BCL6 and NQO2, BCL6 and ORM1, BCL6 and DEFA4, BCL6 and KLRB1, BCL6 and CTSG, BCL6 and LCN2, BCL6 and AZU1, BCL6 and TXN, NQO2 and HLA-DPB1, NQO2 and BCL6, NQO2 and ORM1, NQO2 and DEFA4, NQO2 and KLRB1, NQO2 and CTSG, NQO2 and LCN2, NQO2 and AZU1, NQO2 and TXN, ORM1 and HLA-DPB1, ORM1 and BCL6, ORM1 and NQO2, ORM1 and DEFA4, ORM1 and KLRB1, ORM1 and CTSG, ORM1 and LCN2, ORM1 and AZU1, ORM1 and TXN, DEFA4 and HLA-DPB1, DEFA4 and BCL6, DEFA4 and NQO2, DEFA4 and ORM1, DEFA4 and KLRB1, DEFA4 and CTSG, DEFA4 and LCN2, DEFA4 and AZU1, DEFA4 and TXN, KLRB1 and HLA-DPB1, KLRB1 and BCL6, KLRB1 and NQO2, KLRB1 and ORM1, KLRB1 and DEFA4, KLRB1 and CTSG, KLRB1 and LCN2, KLRB1 and AZU1, KLRB1 and TXN, CTSG and HLA-DPB1, CTSG and BCL6, CTSG and NQO2, CTSG and ORM1, CTSG and DEFA4, CTSG and KLRB1, CTSG and LCN2, CTSG and AZU1, CTSG and TXN, LCN2 and HLA-DPB1, LCN2 and BCL6, LCN2 and NQO2, LCN2 and ORM1, LCN2 and DEFA4, LCN2 and KLRB1, LCN2 and CTSG, LCN2 and AZU1, LCN2 and TXN, AZU1 and HLA-DPB1, AZU1 and BCL6, AZU1 and NQO2, AZU1 and ORM1, AZU1 and DEFA4, AZU1 and KLRB1, AZU1 and CTSG, AZU1 and LCN2, AZU1 and TXN, TXN and HLA-DPB1, TXN and BCL6, TXN and NQO2, TXN and ORM1, TXN and DEFA4, TXN and KLRB1, TXN and CTSG, TXN and LCN2 or TXN and AZU1.

In any embodiment, the levels of the transcripts measured in the assay can be integrated to produce a score, referred to as a “sever or mild” (SoM) infection score, upon which a diagnosis and/or treatment decision may be based. The results can then be integrated to produce a SoM infection score, and the diagnosis/treatment decisions can be based on the score. In some embodiments, the higher the score, the more likely it is that the patient will develop severe symptoms.

In some embodiments, the difference between the geometric mean of the over-expressed genes and the geometric mean of the under-expressed genes can be calculated to provide a score.

In some embodiments, the genes can be placed into modules and the expression of one or at least two (e.g., 3, 4, or 5) or all genes from each module may be tested. Exemplary modules are shown below:
Module 1: NQO2, SLPI, ORM1, KLHL2, BCL2A1, ANXA3, SRGN, TXN, ACSL1, AQP9, ADM, BCL6, TLR2, TLN1, NUCB1, PFKFB4, DOK3, GRN and TYK2.
Module 2: ATP8B4, KIF23, TCEAL9, IGFBP2, BCAT1, BCL2L11, SOCS6, BTBD7, CEP55, HMMR, PRC1, KIF15, TRIP13, CDT1, ELL2, CAMP, OLR1, DEFA4, CEACAM8, LCN2, CTSG and AZU1.
Module 3: MAFB, ANXA2, SCAND1, IFITM1, IFITM3, IFITM2, OASL, UBE2L6, VAMP5, CCL2, CREG1, H1-0, NAPA, FURIN, LAPTM4A, SSR2, RAD23B, FAM8A1, ATG3, VRK2, TMEM123, CASP7 and POMP.
Module 4: HLA-DPB1, DOK2, BANF1, RBM15B, DDB1, LRBA, TRIM28, LTBP3, USP11, ITGB7, EZH1, ARHGAP45, TRAF5, BUB3, SMYD2, TRAF31P3, MAP3K4, CHMP7, PITPNC1, SIDT1, EXOC2, PIK3R1, CCR7, IL7R, EPHX2, TRIB2, FBLN5, KLRB1, KLRG1, PRF1, KLRD1 and PRSS2.

If the genes are divided into modules, then a score can be calculated by summing the scores for module 1 and 2 and then divided by the sum of the scores for module 3 and 4. Other calculations that provide a similar result are envisioned. In some embodiments, the geometric means of the expression of genes from each module can be calculated. A SoM score can be calculated by taking the sum of the geometric means of modules 1 and 2 and dividing that by the sum of the geometric means of modules 3 and 4.

The table below provides several examples of subsets of the genes that can discriminate between severe and mild infections.

| | Severe vs. Non-Severe | Severe vs. Moderate |
|---|---|---|
| Genes | (AUROC) | (AUROC) |
| NQO2, SLPI, ORM1, KLHL2, ANXA3, TXN, AQP9, BCL6, | 0.878 (95% CI 0.855-0.901) | 0.711 (95% CI 0.674-0.748) |
| DOK3, PFKFB4, TYK2, BCL2L11, BCAT1, BTBD7, CEP55, | | |
| HMMR, PRC1, KIF15, CAMP, CEACAM8, DEFA4, LCN2, | | |
| CTSG, AZU1, MAFB, OASL, UBE2L6, VAMP5, CCL2, | | |
| NAPA, ATG3, VRK2, TMEM123, CASP7, DOK2, HLA- | | |
| DPB1, BUB3, SMYD2, SIDT1, EXOC2, TRIB2, KLRB1 | | |
| BCL6, NQO2, DEFA4, CEP55, HMMR, ATG3, VAMP5, | 0.924 (95% CI 0.906-0.943) | 0.801 (95% CI 0.769-0.832) |
| KLRB1, HLA-DPB1, DOK2 | | |
| BCL6, NQO2, DEFA4, CEP55, HMMR, ATG3, VAMP5, | 0.925 (95% CI 0.907-0.943) | 0.798 (95% CI 0.766-0.83) |
| KLRB1, HLA-DPB1 | | |
| BCL6, NQO2, DEFA4, CEP55, HMMR, VAMP5, KLRB1, | 0.922 (95% CI 0.903-0.941) | 0.796 (95% CI 0.764-0.828) |
| HLA-DPB1 | | |
| NQO2, DEFA4, CEP55, HMMR, VAMP5, KLRB1, HLA- | 0.918 (95% CI 0.898-0.937) | 0.791 (95% CI 0.759-0.823) |
| DPB1 | | |
| NQO2, CEP55, HMMB, VAMP5, KLRB1, HLA-DPB1 | 0.914 (95% CI 0.894-0.933) | 0.786 (95% CI 8.753-0.818) |
| NQO2, HMMR, VAMP5, KLRB1, HLA-DPB1 | 0.911 (95% CI 0.898-0.937) | 0.785 (95% CI 0.753-0.818) |
| NQO2, HMMR, VAMP5, HLA-DPB1 | 0.898 (95% CI 0.877-0.919) | 0.776 (95% CI 0.742-0.809) |
| BCL6, NQO2, ORM1, DEFA4, AQP9, GRN, CEP55, | 0.913 (95% CI 0.893-0.932) | 0.808 (95% CI 0.777-0.839) |
| TRIP13, SCAND1, IFITM2, POMP, BTBD7, SOCS6, HLA- | | |
| DPB1, KLRB1, DOK2, ARHGAP45, SSR2, LAPTM4A, | | |
| TYK2 | | |
| NQO2, SCAND1, BCL6, TCEAL9, TRIP13, HLA- | 0.895 (95% CI 0.874-0.917) | 0.793 (95% CI 0.761-0.825) |
| DPB1, MAFB, ATG3, DOK2, EXOC2 | | |
| TXN, NQO2, BCL6, LCN2, ORM1, HLA- | 0.886 (95% CI 0.864-0.908) | 0.786 (95% CI 0.753-0.818) |
| DPB1, DOK2, KLRD1 | | |
| BCL6, POMP, SCAND1, HLA-DPB1, MAFB, DOK2 | 0.871 (95% CI 0.848-0.894) | 0.783 (95% CI 0.751-0.816) |

For example, the severity of infection can be reliably and accurately predicted using: A 42-gene module signature composed of Module 1: NQ2, SLPI, ORM1, KLHL2, ANXA3, TXN, AQP9, BCL6, DOK3, PFKFB4 and TYK2; Module 2: BCL2L11, BCAT1, BTBD7, CEP55, HMMR, PR5C, KIF15, CAMP, CEACAM 8, DEFA4, LCN2, CTSG and AZUL; Module 3: MAFB, OASL, UBE2L6, VAMP5, CCL2, NAPA, ATG3, VRK2, TMEM123, CASP7; Module 4: DOK2, HLA-DPB1, BUB3, SMYD2, SIDT1, EXOC2, TRIB2 and KLRB1.

A 10-gene module signature composed of Module 1: BCL6, NQO2; Module 2: DEFA4, CEP55, HMMR; Module 3: ATG3, VAMP5; Module 4: KLRB1, HLA-DPB1, DOK2.

A 9-gene module signature composed of Module 1: BCL6, NQO2; Module 2: DEFA4, CEP55, HMMR; Module 3: ATG3, VAMP5; Module 4: KLRB1, HLA-DPB1.

An 8-gene module signature composed of: Module 1: BCL6, NQ2; Module 2: DEFA4, CEP55, HMMR; Module 3: VAMP5; Module 4: KLRB1, HLA-DPB1.

A 7-gene module signature composed of: Module 1: NQO2; Module 2: DEFA4, CEP55, HMMR; Module 3: VAMP5; Module 4: KLRB1, HLA-DPB1.

A 6-gene module signature composed of: Module 1: NQO2; Module 2: CEP55, HMMR; Module 3: VAMP5; Module 4: KLRB1, HLA-DPB1.

A 5-gene module signature composed of: Module 1: NQO2; Module 2: HMMR; Module 3: VAMP5; Module 4: KLRB1, HLA-DPB1.

A 4-gene module signature: Module 1: NQO2; Module 2: HMMR; Module 3: VAMP5; Module 4: HLA-DPB1.

A 20-gene signature composed of: upregulated composed of: BCL6, NQO2, ORM1, DEFA4, AQP9, GRN, CEP55, TRIP13, SCAN D1, IFITM2, POMP, BTBD7, SOCS6; downregulated: HLA-DPB1, KLRB1, DOK2, ARHGAP45, SSR2, LAPTM4A, TYK2.

A 10-gene signature composed of: upregulated: NQO2, SCAND1, BCL6, TCEAL9, TRIP13; downregulated: HLA-DPB1, MAFB, ATG3, DOK2, EXOC2.

A 9-gene signature composed of: upregulated: TXN, NQO2, BCL6, LCN2, ORM1; downregulated: HLA-DPB1, DOK2, KLRD1.

A 6-gene signature composed of upregulated: BCL6, POMP, SCAND1; downregulated: HLA-DPB1, MAFB, DOK2.

As noted above, the method should be practiced on RNA obtained from a sample of a patient that has already been infected by a virus. The method can be practiced without knowing exactly which virus the subject has been infected by. However, the subject should be known to be infected by a virus in order for the method to work. In some embodiments, the subject may have been diagnosed as being infected by a virus. The diagnosis may be done by viral isolation and culture, antibody detection (by ELISA, EIA, CLIA, IF, IC IB or IgG avidity testing, etc.), electron microscopy, or through analysis of nucleic acids (e.g., by sequencing, conventional PCR, real-time PCR, RT-PCT, or using an isothermal method such as TMA, NASBA or LAMP). In these embodiments, the patient may be known to be infected by a particular virus (e.g., SARS-CoV-2, Ebola, chikungunya, avian flu, MERS, Zika or dengue, etc.).

The measuring step can be done using any suitable method. For example, the amount of the RNA transcripts in the sample may be measured by RNA-seq (see, e.g., Morin et al BioTechniques 2008 45: 81-94; Wang et al 2009 Nature Reviews Genetics 10: 57-63), RT-PCR (Freeman et al BioTechniques 1999 26:112-22, 124-5), or by labeling the RNA or cDNA made from the same and hybridizing the labeled RNA or cDNA to an array. An array may contain spatially-addressable or optically-addressable sequence-specific oligonucleotide probes that specifically hybridize to transcripts being measured, or cDNA made from the same. Spatially-addressable arrays (which are commonly referred to as “microarrays” in the art) are described in, e.g., Sealfon et al (see, e.g., Methods Mol Biol. 2011; 671:3-34). Optically-addressable arrays (which are commonly referred to as “bead arrays” in the art) use beads that internally dyed with fluorophores of differing colors, intensities and/or ratios such that the beads can be distinguished from each other, where the beads are also attached to an oligonucleotide probe. Exemplary bead-based assays are described in Dupont et al (J. Reprod Immunol. 2005 66:175-91) and Khalifian et al (J Invest Dermatol. 2015 135: 1-5). The abundance of transcripts in a sample can also be analyzed by quantitative RT-PCR or isothermal amplification method such as those described in Gao et al (J. Virol Methods. 2018 255: 71-75), Pease et al (Biomed Microdevices (2018) 20: 56) or Nixon et (Biomol. Det. and Quant 2014 2: 4-10), for example. Many other methods for measuring the amount of an RNA transcript in a sample are known in the art.

The sample of RNA obtained from the subject may comprise RNA isolated from whole blood, white blood cells, PBMCs, neutrophils or buffy coat, for example. In alternative embodiments, the RNA from a nasal swab, a throat swab, or nasal mucous may be analyzed. Methods for making total RNA, polyA+RNA, RNA that has been depleted for abundant transcripts, and RNA that has been enriched for the transcripts being measured are well known (see, e.g., Hitchen et al J Biomol Tech. 2013 24: S43-S44). If the method involves making cDNA from the RNA, then the cDNA may be made using an oligo(d)T primer, a random primer or a population of gene-specific primers that hybridize to the transcripts being analyzed.

In measuring the transcript, the absolute amount of each transcript may be determined, or the amount of each transcript relative to one or more control transcripts, e.g., one or more constitutively expressed transcripts, may be determined. Whether the amount of a transcript is increased or decreased may be in relation to the amount of the transcript (e.g., the average amount of the transcript) in control samples (e.g., in equivalent samples collected from a population of at least 100, at least 200, or at least 500 subjects that do not have severe symptoms).

In some embodiments, the method may comprise providing a report indicating the risk of a subject having severe symptoms, where the subject has been infected by a virus. In some embodiments, this step may involve calculating one or more scores based on the weighted amounts of each of the transcripts, where the one or more scores correlate with the phenotype and can be a number such as a probability, likelihood or score out of 10, for example. In these embodiments, the method may comprise inputting the amounts of each of the transcripts into one or more algorithms, executing the algorithms, and receiving a score for each phenotype based on the calculations. In these embodiments, other measurements from the subject, e.g., whether the subject is male, the age of the subject, white blood cell count, neutrophils count, band count, lymphocyte count, monocyte count, whether the subject is immunosuppressed, and/or whether there are Gram-negative bacteria present, etc., may be input into the algorithm.

In some embodiments, the method may involve creating a report that shows the risk score of the subject, e.g., in an electronic form, and forwarding the report to a doctor or other medical professional to help identify a suitable course of action, e.g., to identify a suitable therapy for the subject. The report may be used along with other metrics as a diagnostic to determine whether the subject has a disease or condition.

In any embodiment, report can be forwarded to a “remote location”, where “remote location,” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like. In certain embodiments, the report may be analyzed by an MD or other qualified medical professional, and a report based on the results of the analysis of the image may be forwarded to the subject from which the sample was obtained.

In computer-related embodiments, a system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining a risk score. The computer processor is coupled to the storage component and configured to execute the instructions stored in the storage component in order to receive patient data and analyze patient data according to one or more algorithms. The display component may display information regarding the diagnosis of the patient.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

### Therapeutic Methods

Therapeutic methods are also provided. In some embodiments, the method may involve identifying a patient as being at high risk of having or developing severe symptoms and then treating the patient accordingly. In these embodiments, the method may comprise:
(b) receiving a report indicating the risk of a subject that been infected by a virus of having severe symptoms, wherein the report is based on the gene expression data obtained by measuring the amount of RNA transcripts encoded by at least two of (e.g., at least 2, at least 3, at least 5, at least 10, at least 15, at least 20, at least 30, at least 40, at least 50 or all of) HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFfTM1, DDB1, BCL2L11, LAPTM4A, K1F23, TYK2, PIK3R1, BANF1, TRIM28, SOCS6, LRBA, ANXA2, IFITM3, CREG1, and NAPA in a sample of RNA obtained from the subject; and
(b) treating the subject based on whether the subject has a high risk of having or developing severe symptoms.

As noted above: (i) increased expression of BCL6, NQO2, ORM1, DEFA4, CTSG, LCN2, AZU1, TXN, CCL2, CEACAM8, AQP9, GRN, CAMP, TLR2, ANXA3, SLPI, KLHL2, CEP55, SRGN, TRIP13, PRC1, TCEAL9, BCAT1, FURIN, ACSL1, HMMR, UBE2L6, CASP7, OLR1, SCAND1, DOK3, KIF15, ATP8B4, IGFBP2, IFITM2, PFKFB4, VAMP5, ELL2, POMP, H1-0, ADM, VRK2, MAFB, CDT1, OASL, TMEM123, TLN1, NUCB1, FAM8A1, BTBD7, ATG3, BCL2A1, IFITM1, BCL2L11, KIF23, SOCS6, ANXA2, IFITM3, CREG1 and NAPA; and (ii) decreased expression of HLA-DPB1, KLRB1, DOK2, KLRG1, KLRD1, EPHX2, EXOC2, PRF1, PRSS23, TRIB2, EZH1, BUB3, ITGB7, SIDT1, RAD23B, ARHGAP45, MAP3K4, USP11, SMYD2, SSR2, IL7R, FBLN5, TRAF5, TRAF31P3, CCR7, LTBP3, CHMP7, PITPNC1, RBM15B, DDB1, LAPTM4A, TYK2, PIK3R1, BANF1, TRIM28 and LRBA increases the risk of the subject will have severe symptoms.

As described above, the levels of these transcripts may be used to calculate a single score, e.g., a number, where the score indicates whether the subject will develop severe symptoms.

As would be apparent, this method may be practiced with any of the subsets of the genes listed above.

In some embodiments, the method may comprise comparing the risk to a threshold or curve, determining that the risk is above a threshold, and administering intensive care or an antiviral therapy to the patient. This care/therapy may be preemptive in some cases since the patient may not yet display severe symptoms at the point at which the test is done.

In some embodiments, the treatment may be administering intensive care to the patient, where the intensive care may comprises one or more of providing supplemental oxygen to the patient, putting the patient on mechanical ventilation, connecting the patient with a device to monitor a bodily function selected from one or more of heart and pulse rate, air flow to the lungs, blood pressure, blood flow, central venous pressure, amount of oxygen in the blood, and body temperature, and adding an intravenous line to the patient. The patient may be admitted to an ICU (intensive care unit).

In some embodiments, the anti-viral therapy may include administering a therapeutic dose of camostat mesylate, nafamostat mesylate, chloroquine phosphate, hydroxychloroquine, cepharanthine/selamectin/mefloquine hydrochloride, remdesivir, N4, hydroxyctidine, lopinavir/ritonavir, umifenovir, favipiravir, oseltamivir or N3 to the subject, e.g., if the patient has COVID-19.

In other embodiments, the antiviral therapy may comprises administering a therapeutic dose of broad-spectrum antiviral agent, an antiviral vaccine, a neuraminidase inhibitor (e.g., zanamivir (Relenza) and oseltamivir (Tamiflu)), a nucleoside analogue (e.g., acyclovir, zidovudine (AZT), and lamivudine), an antisense antiviral agent (e.g., phosphorothioate antisense antiviral agents (e.g., Fomivirsen (Vitravene) for cytomegalovirus retinitis), morpholino antisense antiviral agents), an inhibitor of viral uncoating (e.g., Amantadine and rimantadine for influenza, Pleconaril for rhinoviruses), an inhibitor of viral entry (e.g., Fuzeon for HIV), an inhibitor of viral assembly (e.g., Rifampicin), or an antiviral agent that stimulates the immune system (e.g., interferons). Exemplary antiviral agents include Abacavir, Aciclovir, Acyclovir, Adefovir, Amantadine, Amprenavir, Ampligen, Arbidol, Atazanavir, Atripla (fixed dose drug), Balavir, Cidofovir, Combivir (fixed dose drug), Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type Ill, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), or Zidovudine to the patient.

“Severe” symptoms are well known to medical practitioners. These symptoms may vary from virus to virus, and may include high fever, severe cough, and shortness of breath, which often indicates pneumonia, neurological symptoms, and or gastrointestinal (GI) symptoms (COVID-19), high fever, rash, debilitating headache, joint and muscle pain (Zika), difficulty breathing and shortness of breath, persistent pain or pressure in the chest or abdomen, persistent dizziness, confusion, inability to arouse, seizures, severe muscle pain, and/or severe weakness or unsteadiness (flu), debilitating headache, muscle pain, joint swelling, and/or a rash (chikungunya) and high fever. severe aches and pains (such as severe headache, muscle and joint pain, and abdominal pain) debilitating weakness and fatigue and gastrointestinal symptoms such as diarrhea and vomiting (Ebola).

Methods for administering and dosages for administering the therapeutics listed above are known in the art or can be derived from the art.

### Kits

Also provided by this disclosure are kits for practicing the subject methods, as described above. In some embodiments, the kit may reagents for measuring the amount of RNA transcripts encoded by at least 2, at least 3, at least 5, at least 10, at least 15, at least 20, at least 30 or all of HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFfTM1, DDB1, BCL2L11, LAPTM4A, K1F23, TYK2, PIK3R1, BANF1, TRIM28, SOCS6, LRBA, ANXA2, IFfTM3, CREG1, and NAPA. In some embodiments, the kit may comprise, for each RNA transcript, a sequence-specific oligonucleotide that hybridizes to the transcript. In some embodiments, the sequence-specific oligonucleotide may be biotinylated and/or labeled with an optically-detectable moiety. In some embodiments, the kit may comprise, for each RNA transcript, a pair of PCR primers that amplify a sequence from the RNA transcript, or cDNA made from the same. In some embodiments, the kit may comprise an array of oligonucleotide probes, wherein the array comprises, for each RNA transcript, at least one sequence-specific oligonucleotide that hybridizes to the transcript. The oligonucleotide probes may be spatially addressable on the surface of a planar support, or tethered to optically addressable beads, for example.

The various components of the kit may be present in separate containers or certain compatible components may be precombined into a single container, as desired.

In addition to the above-mentioned components, the subject kit may further include instructions for using the components of the kit to practice the subject method.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

The four pandemic viral outbreaks in the last decade have underscored the lack of a generalizable diagnostic and prognostic tests in our pandemic preparedness. Tests that are readily usable in clinical practice, irrespective of novel or re-emerging virus, for distinguishing patients at higher risk of severe outcome from those with mild infection could help to avoid overwhelming healthcare systems worldwide. The following data were integrated: 4,780 blood transcriptome profiles from patients (<12 months to 73 years) with one of 16 viral infections across 34 independent cohorts from 18 countries, and scRNA-seq profiles of 264,000 immune cells from 71 samples across 3 independent cohorts to identify host response modules associated with severity of viral infection irrespective of virus. Despite the biological, clinical, and technical heterogeneity across these cohorts, it was found that a myeloid cell-dominated conserved host immune response to viral infection is associated with severity, and identifies distinct trajectories for mild or severe outcomes in patients with viral infection, irrespective of infecting virus. Analyses of these trajectories showed increased hematopoiesis, myelopoiesis, and myeloid-derived suppressor cells, and reduced NK and T cells are associated with increased severity of viral infections across all cohorts, irrespective of the infecting virus. It was found that interferon response is decoupled from protective host response module in patients with severe viral infection, but not in those with mild infection. Finally, a SoM score was defined using these modules that accurately distinguish patients with mild or moderate viral infections from those with severe outcomes. Together, these findings offer crucial insights into the underlying immune dynamics of severity of viral infection, and identify factors that may influence infection outcomes.

### Results

### Data Collection, Curation, and Preprocessing

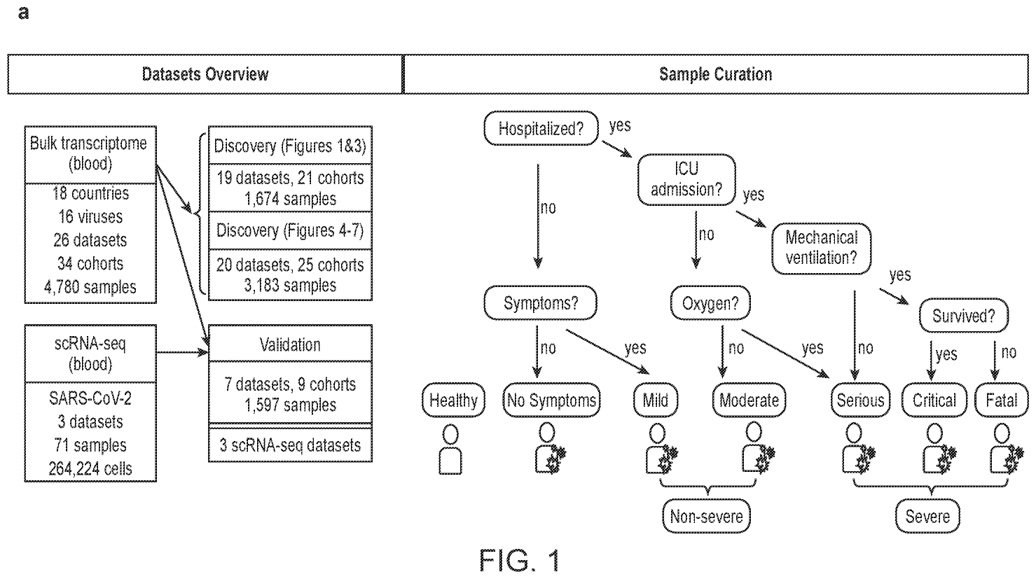

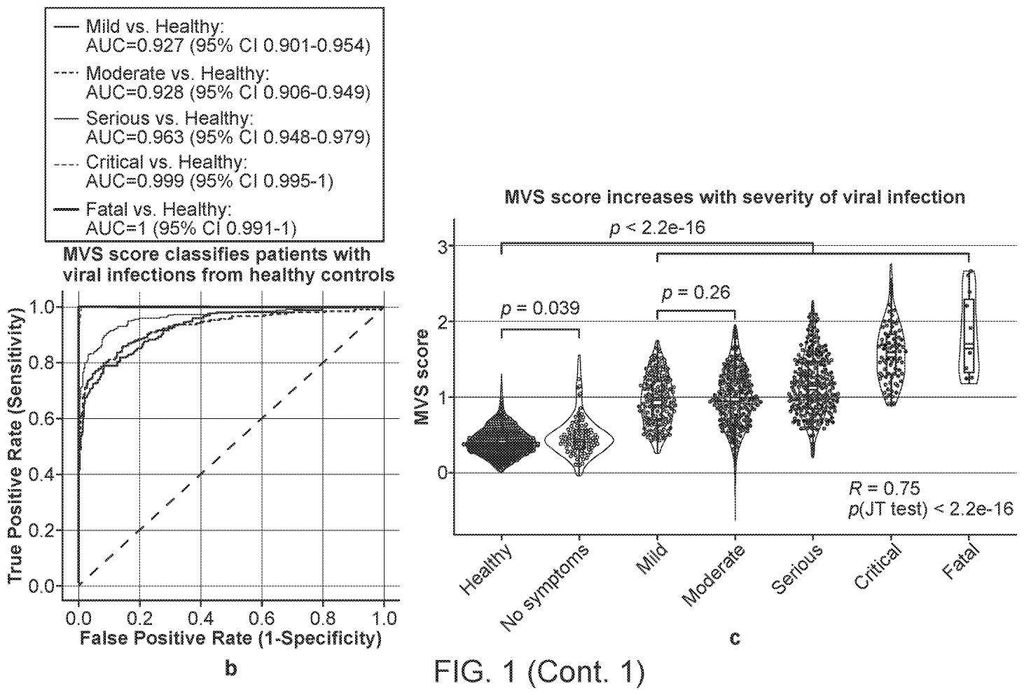

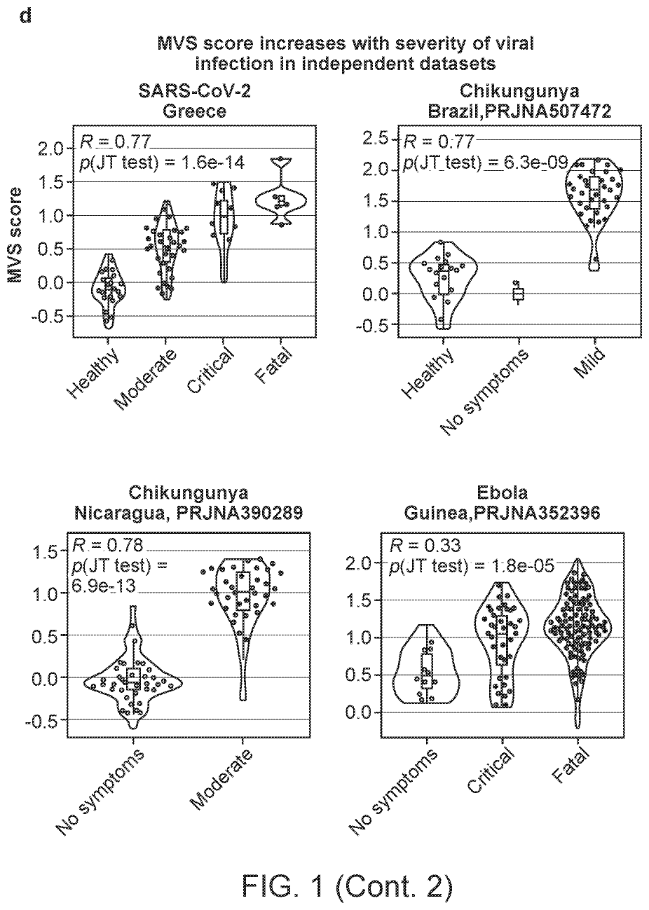

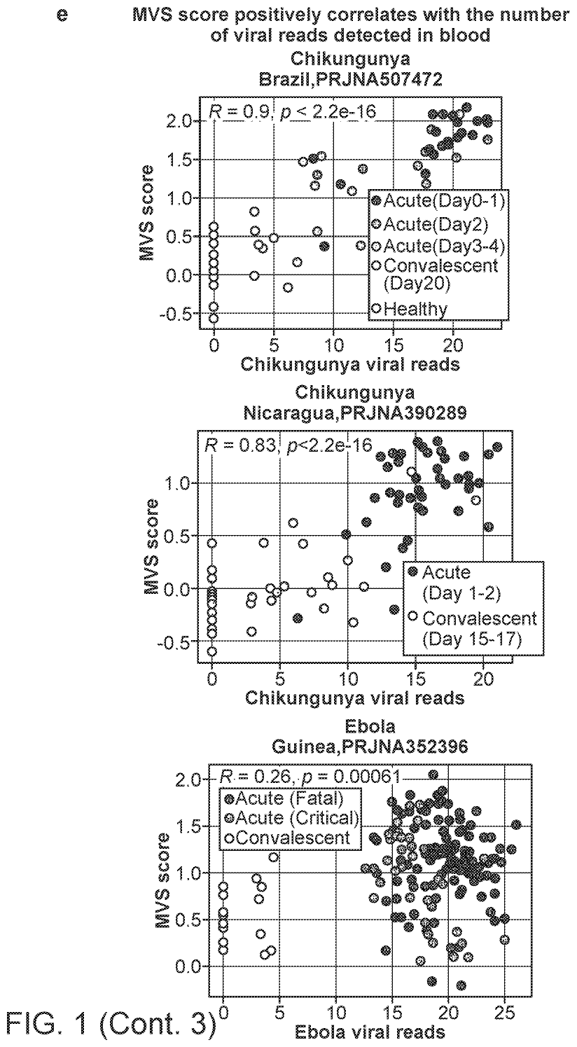

Public repositories (Gene Expression Omnibus, ArrayExpress, European Nucleotide Archive, and Sequence Read Archive) for transcriptome profiles of peripheral blood samples from patients with viral infection were searched. All datasets used to discover the MVS previously were excluded to ensure all cohorts analyzed in the current study were independent. Identified 34 independent cohorts within 26 datasets composed of 4,780 samples from patients across 18 countries infected with at least one of 16 viruses (FIG. 1) were identified. A cohort was defined as a comparable group of individuals within a dataset, where each dataset has a unique GEO identifier and may contain multiple cohorts. For example, the dataset GSE73072 contains seven cohorts of individuals challenged with one of three viruses. Overall, these cohorts included a broad spectrum of biological, clinical, and technical heterogeneity represented by blood samples profiled from children and adults infected with a virus using either microarray or RNA sequencing (Methods).

A standardized severity category was assigned to each of the 4,780 samples (FIG. 1A and Methods). Briefly, samples from individuals with no viral infection and no other disease were assigned to the “healthy” category. Samples from asymptomatic individuals with confirmed acute or convalescent viral infection were assigned to the “no symptoms” category. Next, the symptomatic patients with viral infections were divided into those who were hospitalized and those who were not. Patients with viral infection that were not hospitalized and managed as outpatients were categorized as “mild.” The hospitalized patients with viral infections were further divided based on whether they were admitted to the intensive care unit (ICU) or not. Hospitalized patients admitted to the general wards without requiring supplemental oxygen were assigned to the “moderate” category; those on the general wards who required oxygen or those admitted to the ICU without mechanical ventilation or inotropic support were assigned to the “serious” category, and those admitted to the ICU with mechanical ventilation or inotropic support were assigned to the “critical” category. Patients who died were assigned to the “fatal” category. For cohorts that lacked sample-level severity data, the same severity category was assigned to each sample based on the cohort description. Finally, two additional broader categories were defined: “non-severe,” which included patients with mild and moderate viral infection, and “severe,” which included patients with serious, critical, and fatal viral infection (FIG. 1A).

MVS Represents a Conserved Host Response to Viral Infections and is Associated with Severity

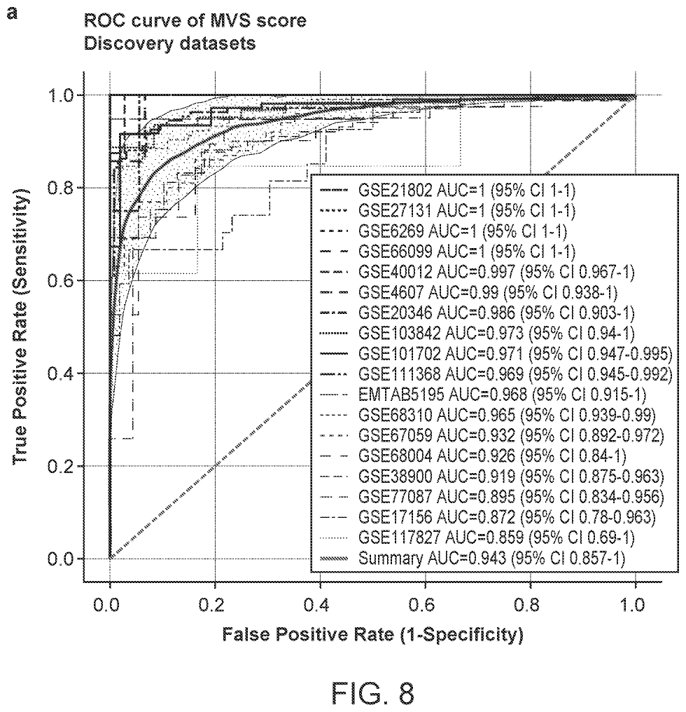

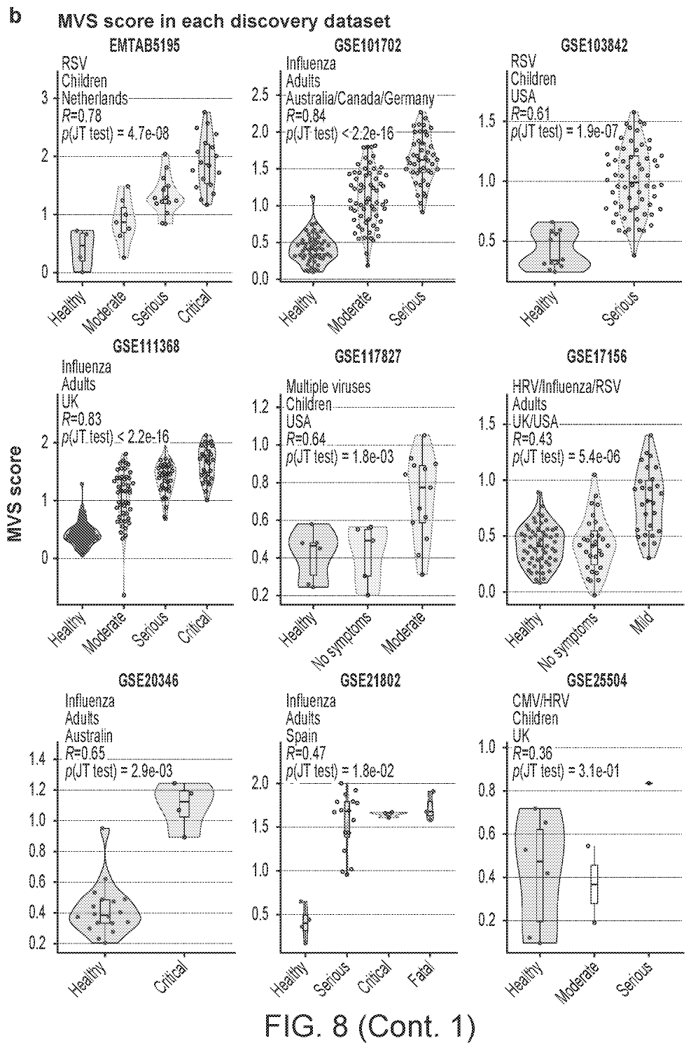

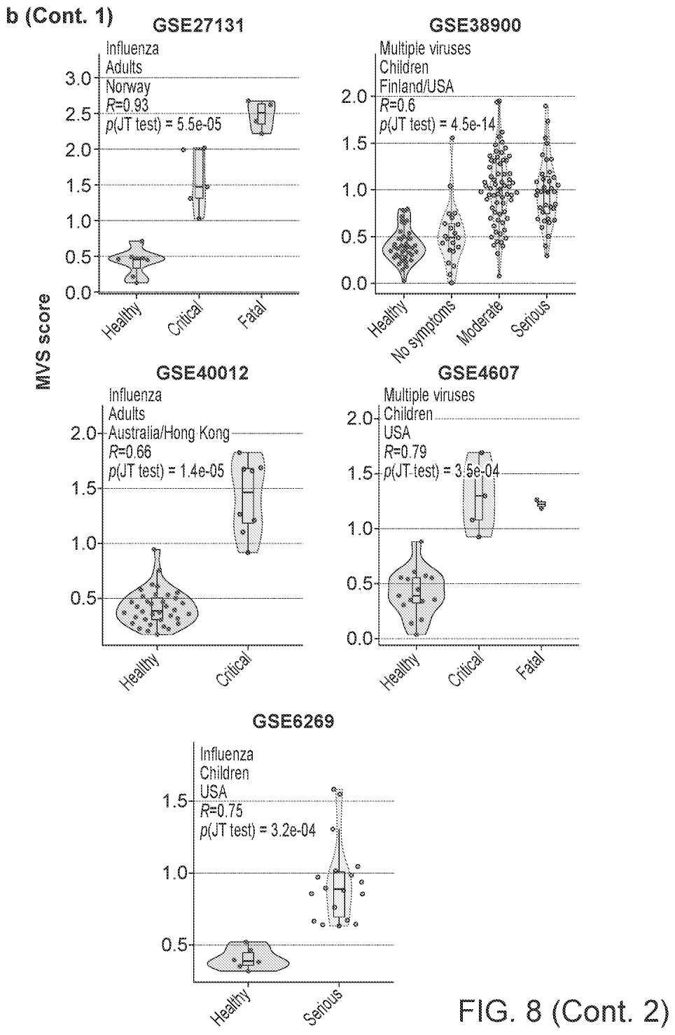

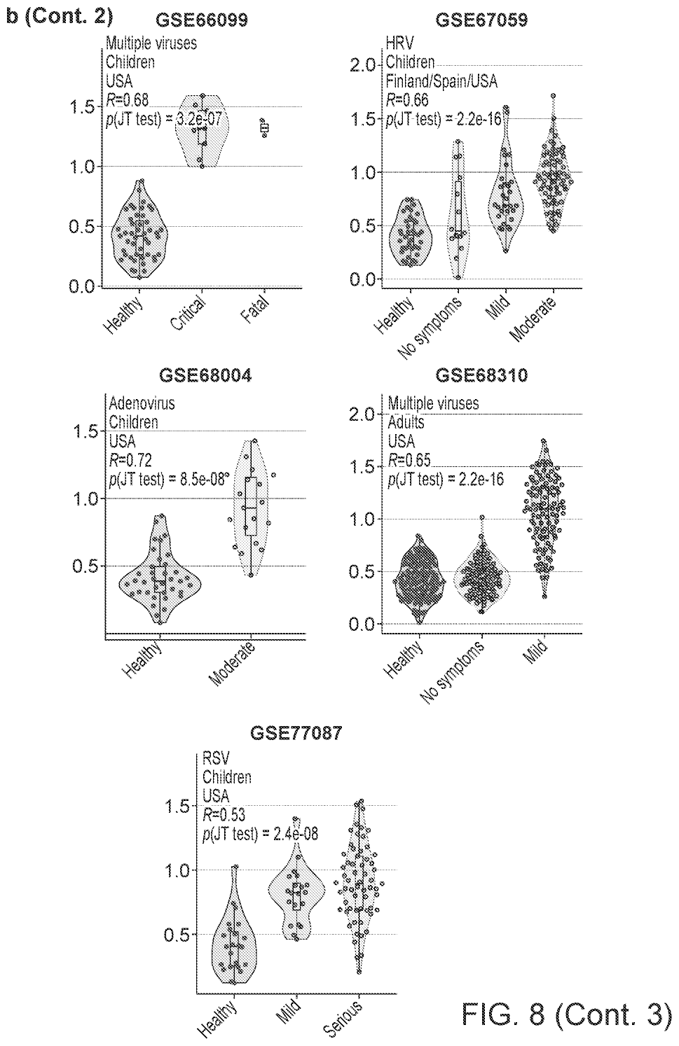

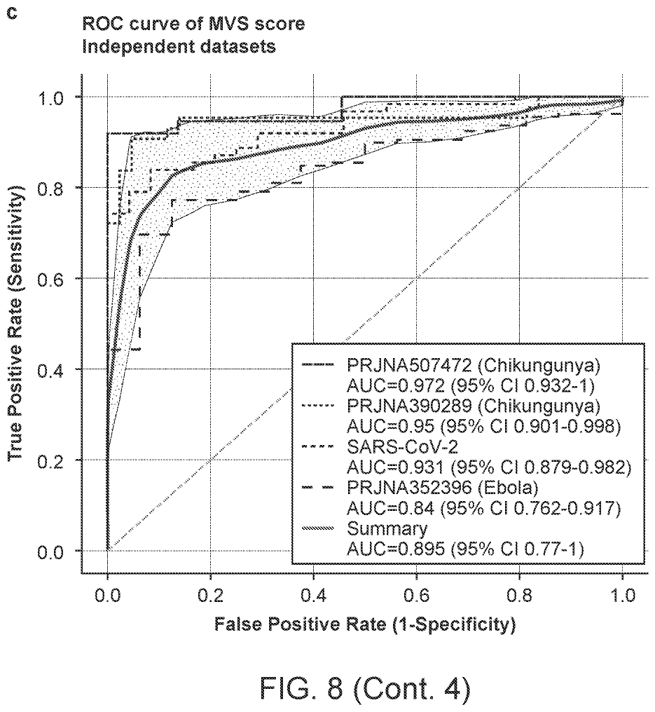

To test the hypothesis that a conserved host response to viral infections, represented by the MVS score, is associated with severity, transcriptome profiles of 1674 blood samples (663 healthy, 167 asymptomatic or convalescent, 181 mild, 286 moderate, 286 serious, 80 critical, and 11 fatal) from 21 cohorts across 19 independent datasets were co-normalized using COCONUT, which removes inter-dataset batch effects while remaining unbiased to the diagnosis of the diseased patients (FIG. 1A and Methods) (Sweeney et al., 2016b). The majority of patients in these 19 datasets were infected with adenovirus, influenza, rhinovirus (HRV), or respiratory syncytial virus (RSV). The MVS score accurately distinguished patients with viral infections from healthy controls across all datasets as well as in individual datasets (FIG. 1B and FIG. 8A). The area under the receiver operating characteristics (AUROC) curves increased with severity (0.925<AUROC≤1), further suggesting that a conserved host response is associated with the severity of viral infections.

Therefore, the MVS score was correlated with standardized severity categories and found a significant correlation between the MVS score and the severity of viral infection (r=0.75, p<2.2e-16; FIG. 1C). The MVS score was significantly higher in all infected patients compared to healthy controls (p<2.2e-16), irrespective of symptoms, severity, and virus (FIG. 1C). In asymptomatically infected or convalescent patients, the MVS score was marginally higher than in healthy controls (p=0.039). The MVS score was not statistically different between patients with mild and moderate severity (p=0.26). Importantly, across all datasets except one, irrespective of virus, geography, or age, the MVS score was significantly correlated with viral infection severity (0.43≤R≤0.93; p<0.02) (FIG. 8B). Furthermore, in 405 samples from patients infected with one of three viruses (SARS-CoV-2, Ebola, chikungunya) across 4 datasets that were not COCONUT co-normalized, the MVS score was correlated with severity (0.33≤R≤0.78; P≤1.8e-05; FIG. 1D) and accurately distinguished patients with viral infections from healthy controls in each dataset (0.84≤R≤0.972; FIG. 8C). In three independent datasets of blood samples from patients with either chikungunya or Ebola infection, profiled using RNA-seq, sequencing reads from the corresponding viral RNA were detected (Methods). In each of these three datasets, the MVS score significantly correlated with the number of viral reads detected in blood (p≤6.1e-4; FIG. 1E). Further, in each dataset, both the number of viral reads in blood and the MVS score decreased as patients progressed from acute infection to convalescence.

Collectively, these results demonstrate that a conserved host response to viral infections, represented by the MVS, is correlated with the severity of viral infection and the number of viral reads detected in blood samples from patients, irrespective of clinical, biological, or technical heterogeneity or the infecting virus.

Myeloid Cells are the Primary Source of MVS that Correlate with the Severity of Viral Infection

Next, to gain a mechanistic understanding of the conserved host response to a viral infection, whether the MVS score is associated with specific immune cell types was investigated. Three single-cell RNA-seq (scRNA-seq) datasets consisting of 264,224 immune cells from 71 PBMC samples (50 SARS-CoV-2, 17 healthy, 2 influenza, 2 RSV) from 54 individuals across three independent datasets (Seattle, Atlanta, Stanford) (Arunachalam et al., 2020; Su et al., 2020; Wilk et al., 2020) were integrated. The Seattle Cohort profiled 135,420 immune cells from 39 PBMC samples of healthy controls and patients with SARS-CoV-2 infection (6 healthy, 1 mild, 8 moderate, 17 serious, 7 critical) using CITE-seq. The patients with SARS-CoV-2 infection in the Seattle Cohort were profiled at two time points: (1) near the time of a positive clinical diagnosis and (2) a few days later. Collectively, these three cohorts included clinical, biological, and technical heterogeneity at a single-cell level. The Atlanta Cohort profiled 84,083 immune cells from 18 PBMC samples of healthy controls and patients infected with one of 3 viruses (5 healthy, 1 moderate influenza, 1 serious influenza, 2 serious RSV, 2 convalescent SARS-CoV-2, 3 moderate SARS-CoV-2, 3 serious SARS-CoV-2, 1 fatal SARS-CoV-2) using CITE-seq. Finally, the Stanford Cohort profiled 44,721 immune cells from 14 PBMC samples of healthy controls and patients with SARS-CoV-2 infection (6 healthy, 1 moderate, 3 serious, 3 critical, 1 fatal) using Seq-Well.

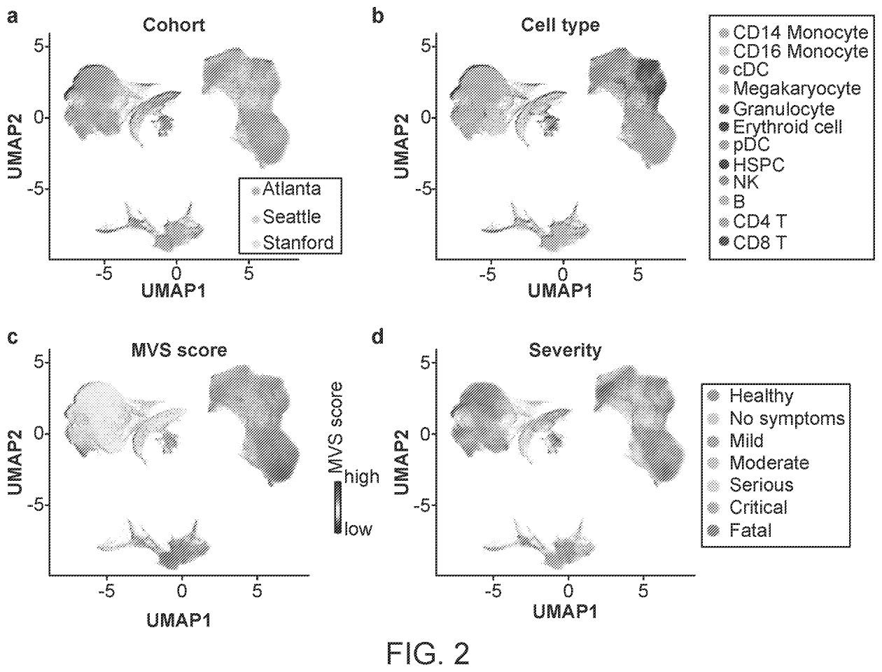

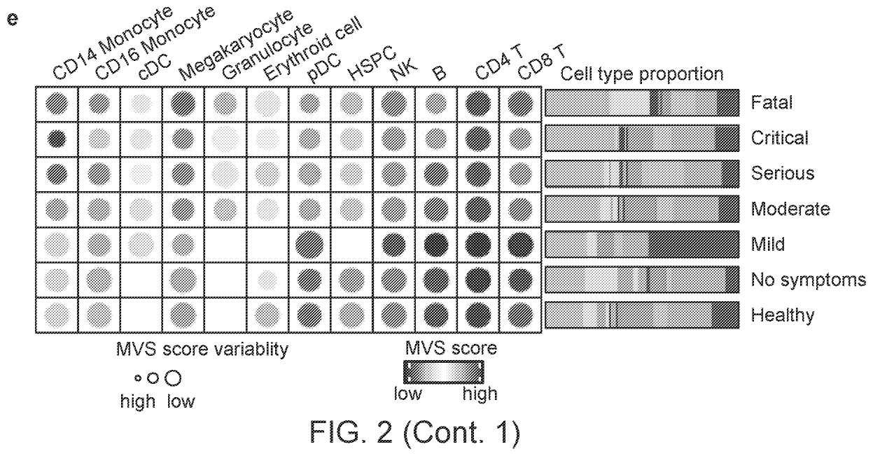

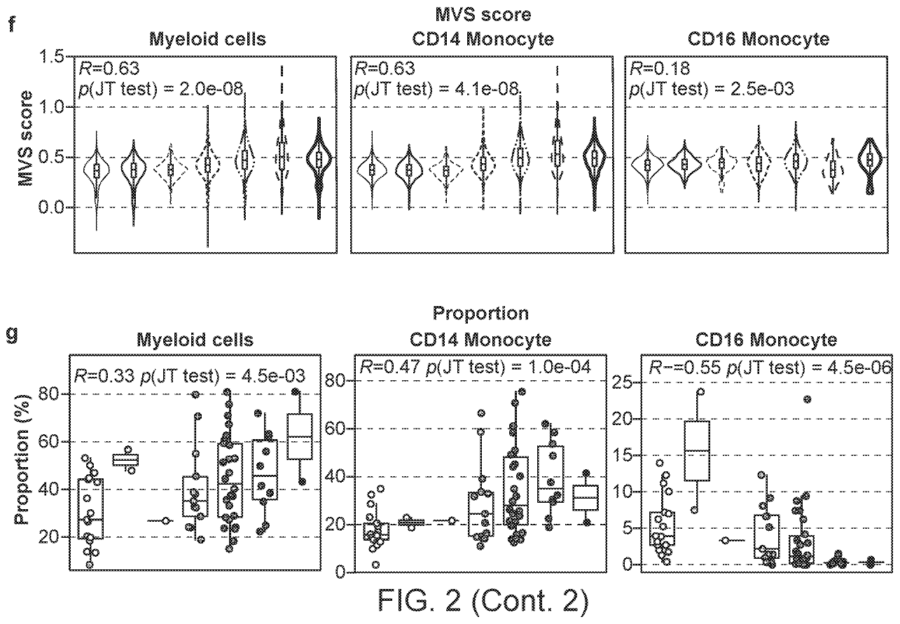

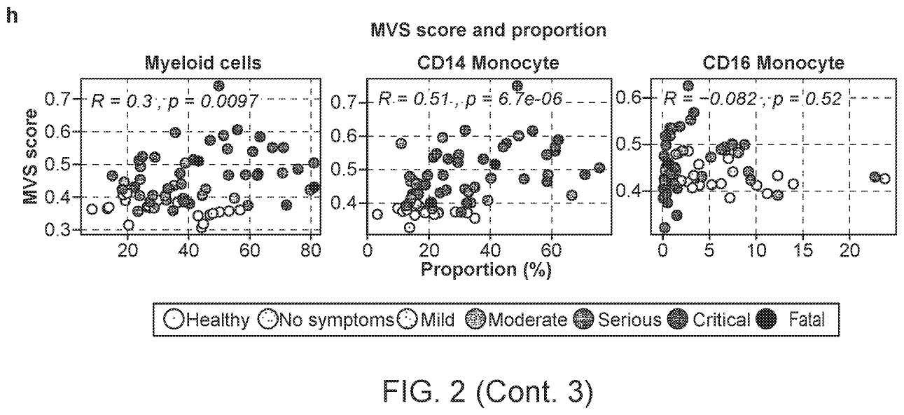

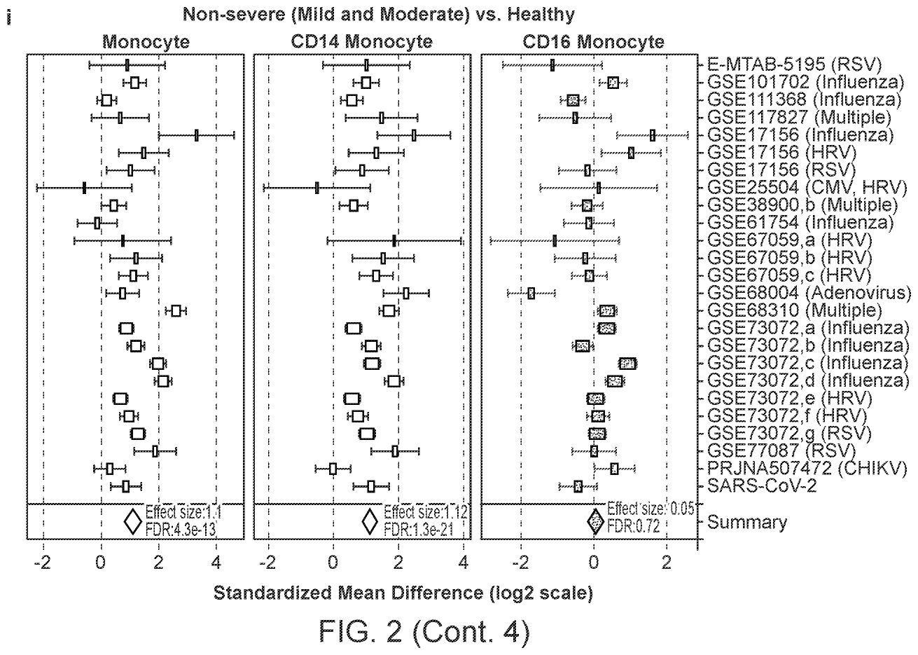

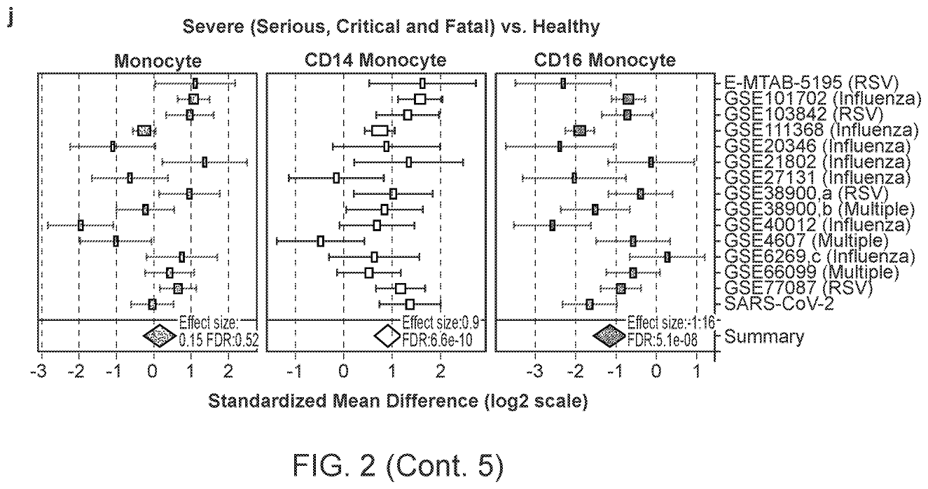

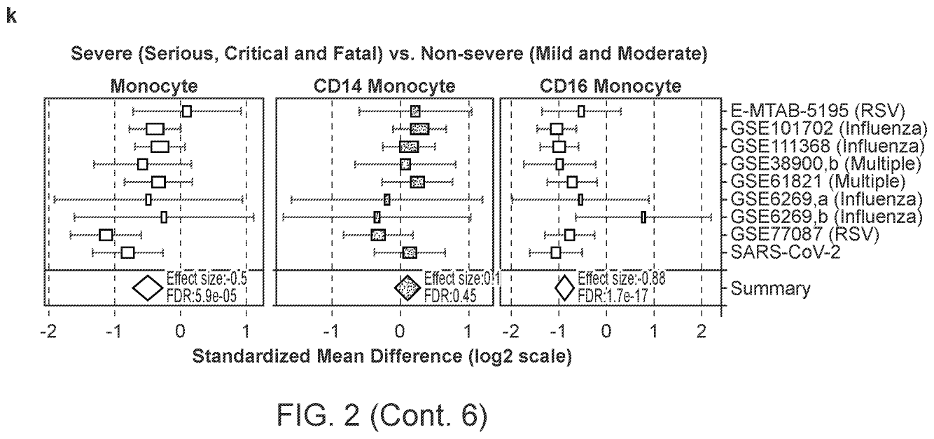

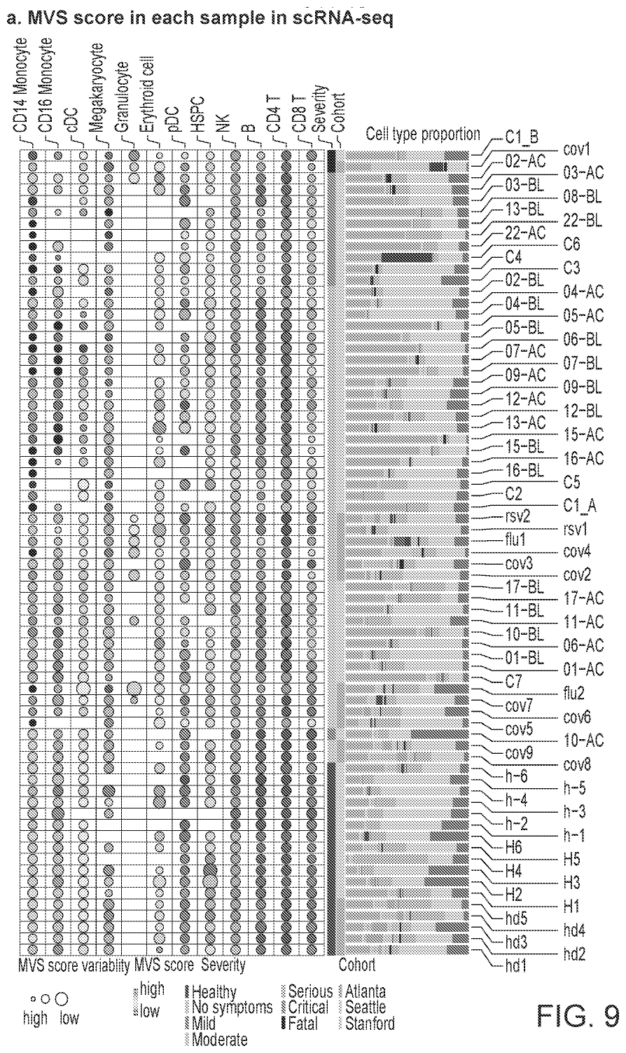

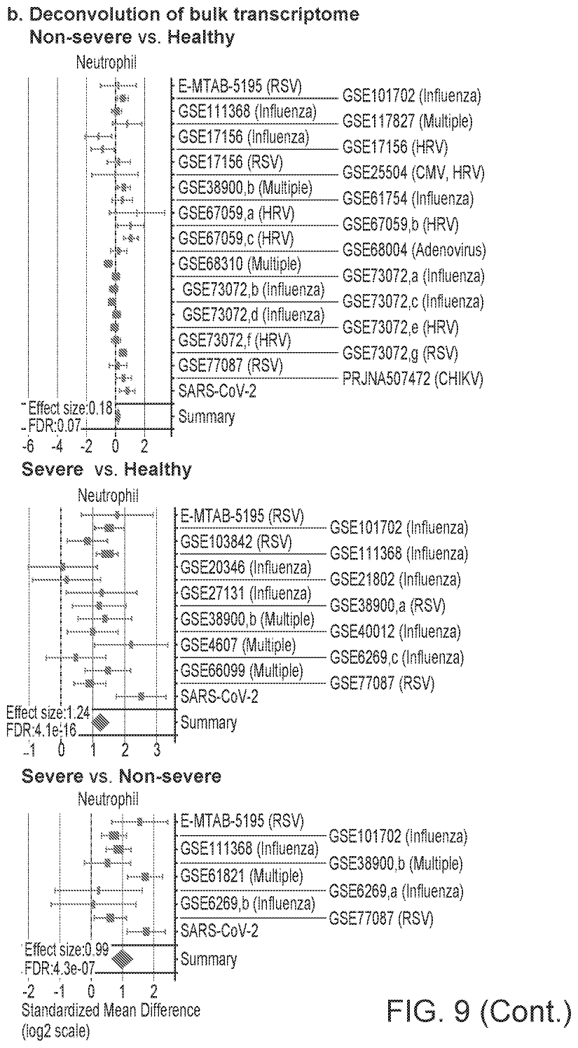

The three scRNA-seq cohorts were integrated using Seurat (Satija et al., 2015), and visualized the data in a low dimensional space using Uniform Manifold Approximation and Projection (UMAP) (FIG. 2A-D). Immune cells across the three cohorts clustered into myeloid cells (monocytes, myeloid dendritic cells, granulocytes, etc.), T and NK cells, and B cells (FIG. 2A-B). The MVS score was substantially higher in myeloid cells from hospitalized patients with viral infection (FIG. 2C-E and FIG. 9A) and positively correlated with the severity of viral infection in myeloid cells at the single-cell level (R=0.63, p=2e-08), which was driven by CD14+ monocytes (R=0.63, p=4.1e-08) compared to CD16+ monocytes (R=0.18, p=2.5e-03) (FIG. 2F). Further, proportions of myeloid cells increased with severity (R=0.33, p=4.5e-03), which was also driven by increased proportions of CD14+ monocytes (R=0.47, p=1e-04). In contrast, proportions of CD16+ monocytes decreased with increasing severity of viral infections (R=−0.55, p=4.5e-06) (FIG. 2G). Finally, the MVS score in myeloid cells at the single-cell level and proportions of myeloid cells were positively correlated (R=0.3, p=0.0097), which was also driven by CD14+ monocytes (R=0.51, p=6.7e-06) but not CD16+ monocytes (FIG. 2H). Collectively, these results demonstrated that in response to a viral infection, proportions of myeloid cells, primarily CD14+ monocytes, increase with severity along with the conserved host response to viral infections represented by the MVS at a single-cell level.

Next, it was investigated whether these changes in CD14+ and CD16+ monocytes were also observed in patients with viral infections at the bulk transcriptome level. We performed in silico cellular deconvolution of blood transcriptome profiles of 4357 patient samples from 32 independent cohorts using immunoStates (Bongen et al., 2018; Chowdhury et al., 2018; Scott et al., 2019; Vallania et al., 2018) to estimate proportions of 25 immune cell subsets in each sample. Then, three multi-cohort analyses were performed to compare changes in immune cell proportions in (1) non-severe viral infections compared to healthy controls, (2) severe viral infections compared to healthy controls, and (3) severe compared to non-severe viral infections.

In line with the scRNA-seq analysis, proportions of total monocytes were significantly higher in patients with non-severe viral infections compared to healthy controls (μS=1.10, FDR=4.33e-13), but were not significantly higher in those with severe viral infections (FIG. 2I-J). Further analysis of monocytes found the proportion of CD14+ monocytes increased significantly in patients with non-severe (ES=1.12, FDR=1.30e-21) and severe (ES=0.9, FDR=6.56e-10) viral infections compared to healthy controls, but were not significantly different between patients with non-severe and severe viral infections (FIG. 2I-K). In contrast, and in line with the scRNA-seq analysis, proportions of CD16+ monocytes were significantly lower in patients with severe viral infections compared to healthy controls (ES=−1.16, FDR=5.13e-08) and those with non-severe viral infections (ES=−0.88, FDR=1.73e-17) (FIG. 2J-K), but were unchanged in non-severe patients compared to healthy controls (FIG. 2I).

Cellular deconvolution analysis also found the proportions of neutrophils were significantly higher in patients with severe viral infections compared to healthy controls (ES=1.24, FDR=4.12e-16) and those with non-severe viral infection (ES=0.99, FDR=4.33e-07) (FIG. 9B). These results were in line with the scRNA-seq data, where granulocytes were detected only in patients with moderate through fatal viral infections (FIG. 2E, FIG. 9A). These cells are likely low-density immature granulocytes typically found in patients with sepsis. These results also demonstrated the complementarity of in silico deconvolution to scRNA-seq, which does not allow the profiling of neutrophils.

Collectively, the integrated analyses of scRNA-seq from 3 cohorts of 71 PBMC samples and in silico cellular deconvolution of bulk transcriptome profiles from 4357 samples across 32 independent cohorts using immunoStates showed that the conserved host response to viral infections is predominantly from myeloid immune cells. It also found that proportions of CD14+ monocytes increased and CD16+ monocytes decreased with increased severity of viral infection.

MVS Identifies Distinct Clusters of Patients with Non-Severe and Severe Viral Infections

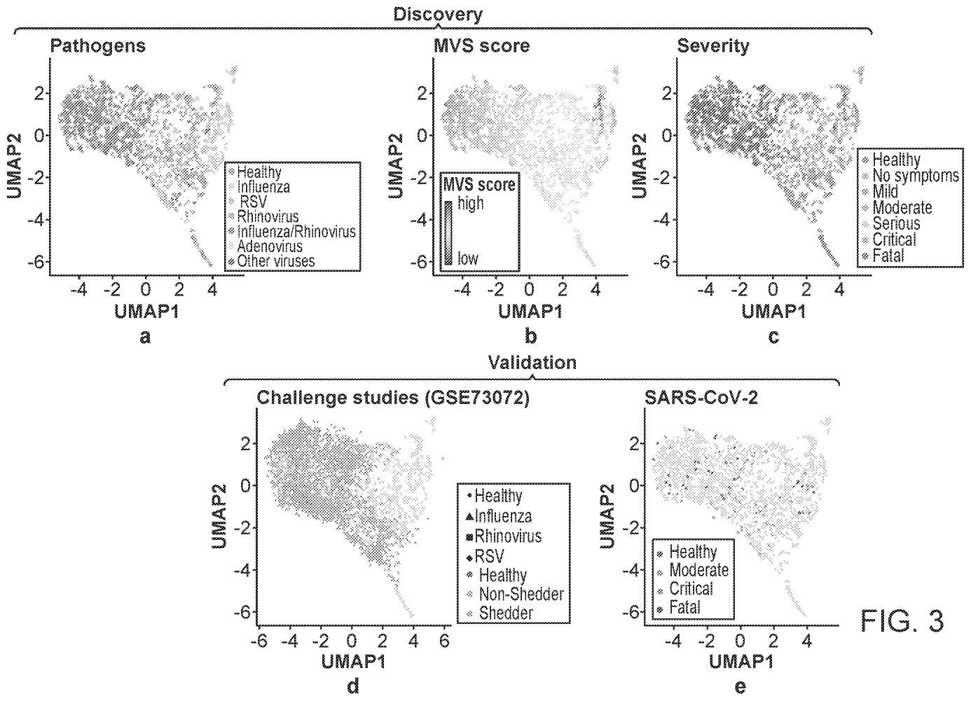

Despite the consistently significant correlation between the MVS score and severity of viral infection, there was a substantial overlap in the MVS score between patients with non-severe and severe viral infections (FIG. 1C). However, low dimensional visualization of 1674 co-normalized samples using UMAP showed that healthy controls were distinct from patients with viral infections irrespective of the infecting virus (FIG. 3A). Importantly, the MVS score increased along the first UMAP component (UMAP1; FIG. 3B), while patients with mild viral infections were clustered separately from those with severe viral infections along the second UMAP component (UMAP2; FIG. 3C). The robustness of the clusters observed in UMAP was further validated by mapping 8 independent cohorts consisting of 2,604 samples from patients with one of 4 viral infections to the same low dimensional space (FIGS. 3D and 3E). All but one of these 8 cohorts were challenge studies, where 129 healthy individuals were inoculated with either influenza (1,465 samples from 70 subjects), RSV (419 samples from 20 subjects), or HRV (634 samples from 39 subjects). Each of the infected subjects in these challenge studies had asymptomatic or mild infection. When mapped to the UMAP space created using the 1,674 samples, samples from the challenge studies clustered with mild viral infections (FIG. 3D). 86 of the samples (24 healthy controls, 62 SARS-CoV-2) was also mapped, which were profiled using RNA-seq and included patients with moderate, critical, and fatal SARS-CoV-2 infection (FIG. 3E). Patients with critical and fatal SARS-CoV-2 infection mapped to the region enriched for patients with critical and fatal viral infections, whereas patients with moderate SARS-CoV-2 infection mapped to the region enriched for patients with non-severe viral infections (FIG. 3E), again demonstrating that the host response to viral infection is conserved and associated with severity, irrespective of the virus. Collectively, this observation suggested that a distinct subset of genes in the MVS may be differentially associated with the severity of viral infection.

Hospitalized Patients with Viral Infection Follow a Different Trajectory from Non-Hospitalized Patients with Viral Infection

Based on low dimensional mapping of samples, it was hypothesized that patients with mild and severe viral infections follow different trajectories. Each sample in our analysis represents a snapshot of the host response to viral infection that spans from recognizing the presence of a virus to its elimination. This is analogous to cellular differentiation analysis using single-cell profiling data, where each cell represents a snapshot along the differentiation trajectory. Therefore, we tested our hypothesis by adapting tSpace (Dermadi et al., 2020), a method for identifying cellular differentiation trajectories using single-cell data, to identify disease trajectories using bulk RNA data. The modified method may be referred to as ‘disease space’ (dSpace) (Methods).

Because the viral challenge cohorts included a large number of longitudinal samples that can aid in a more accurate inference of the host response trajectories, four of the seven challenge studies (1,509 samples across 2 influenza, 1 HRV, and 1 RSV studies) were randomly selected and co-normalized them with 1674 samples from 19 datasets using COCONUT. Overall, dSpace was applied to 3,183 COCONUT co-normalized samples from 25 independent cohorts. All challenge studies when inferring the disease trajectories were not included to avoid introducing class imbalance because subjects in the challenge studies only had mild viral infections. These left-out challenge studies were used for validation of the inferred trajectories.

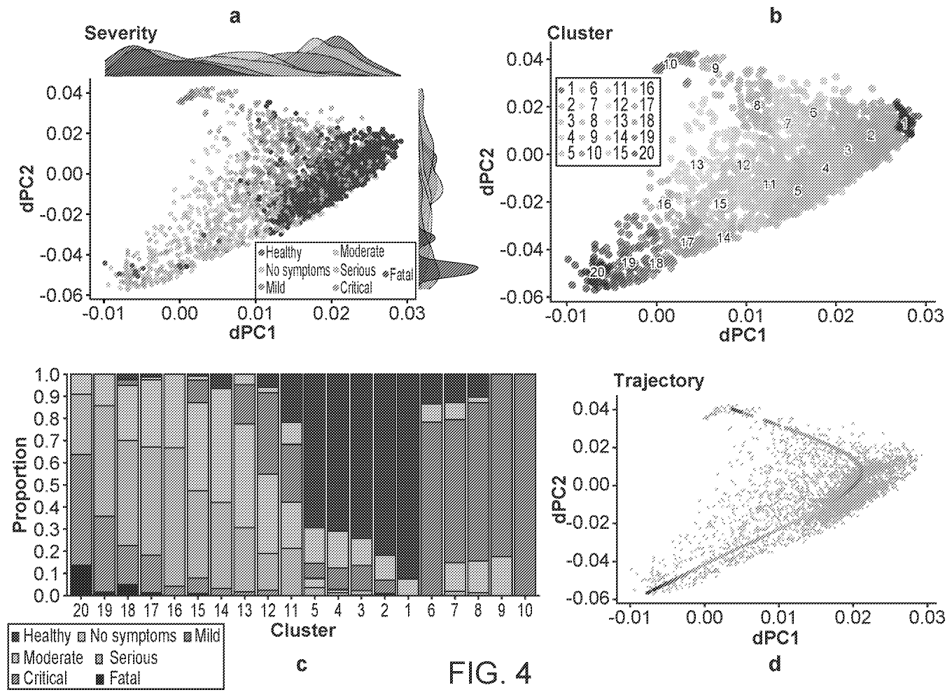

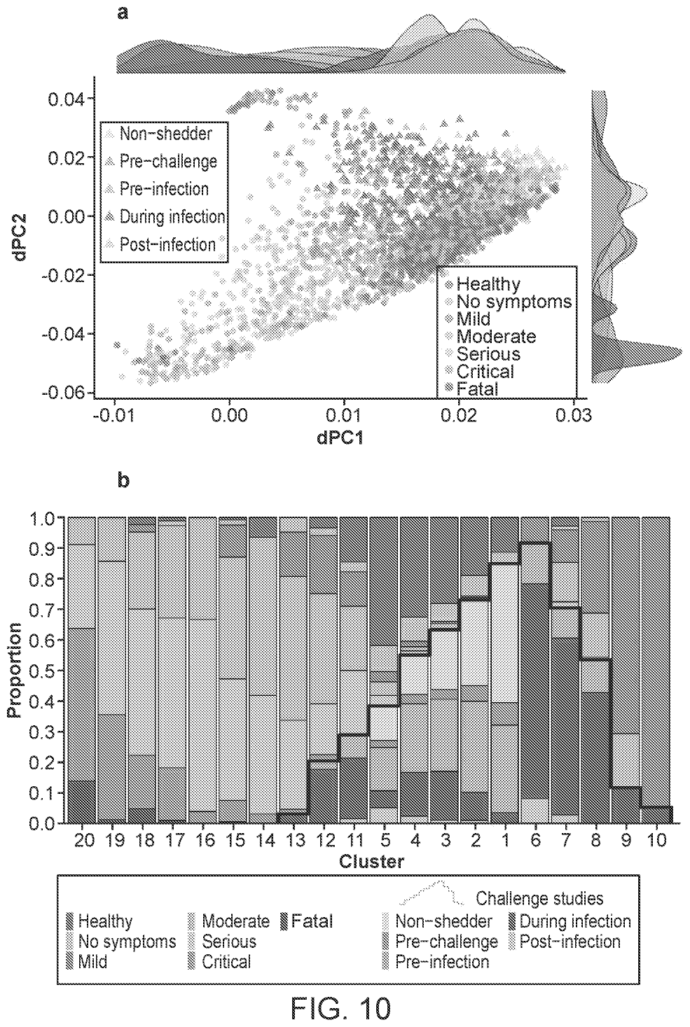

The first principal component of dSpace (dPC1) correlated with the severity of viral infection, whereas the second component (dPC2) distinguished hospitalized patients with viral infections from non-hospitalized patients with mild infections (FIG. 4A). Importantly, participants from the influenza, RSV, and HRV challenge studies clustered almost exclusively with patients with mild infection (FIG. 10A).

Next, samples were clustered using the disease space matrix, and used the resulting clusters to isolate trajectories associated with the severity of viral infection (Methods). 20 clusters were identified that identified 3 groups such that one category of samples dominated (FIG. 4B): clusters 1-5, in which healthy controls and asymptomatically infected or convalescent patients accounted for >80% of samples; clusters 6-10, in which patients with mild viral infection accounted for >68% of samples, and clusters 13-20, in which hospitalized patients with moderate, serious, critical, or fatal viral infections accounted for >77% of samples (FIG. 4C). Clusters 11 and 12 were heterogeneous as no one group of samples dominated them. Strikingly, 1,507 out of 1,509 samples (99.9%) from the influenza, RSV, and HRV challenge studies fell within clusters 1-12 (Figure S3B FIG. 10B), demonstrating the robustness of the clusters defined using dSpace. A principal trajectory line was fit to the dSpace matrix, which consisted of healthy patients in the center and two divergent trajectories: one dominated by patients with mild viral infection and the other dominated by hospitalized patients with viral infection (FIG. 4D, Methods). Hitherto, these trajectories are referred to as “mild trajectory” and “severe trajectory”. Collectively, trajectory analysis using dSpace demonstrated that hospitalized patients with viral infection follow a different trajectory than those with mild infections compared to healthy controls, irrespective of the infecting virus.

Proportions of NK Cells and the Expression of NK Cell-Specific Genes are Negatively Correlated with the Severity of Viral Infection

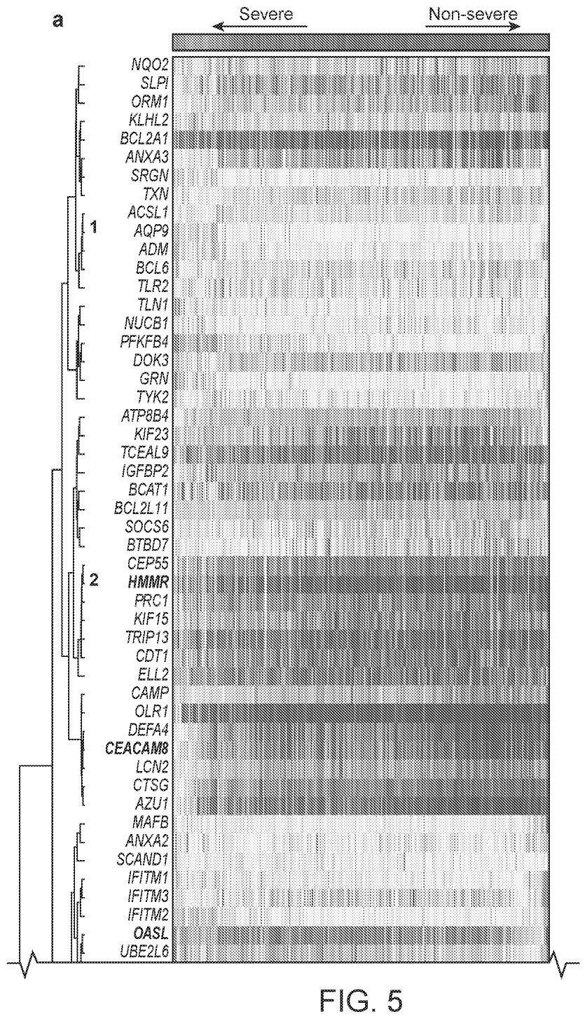

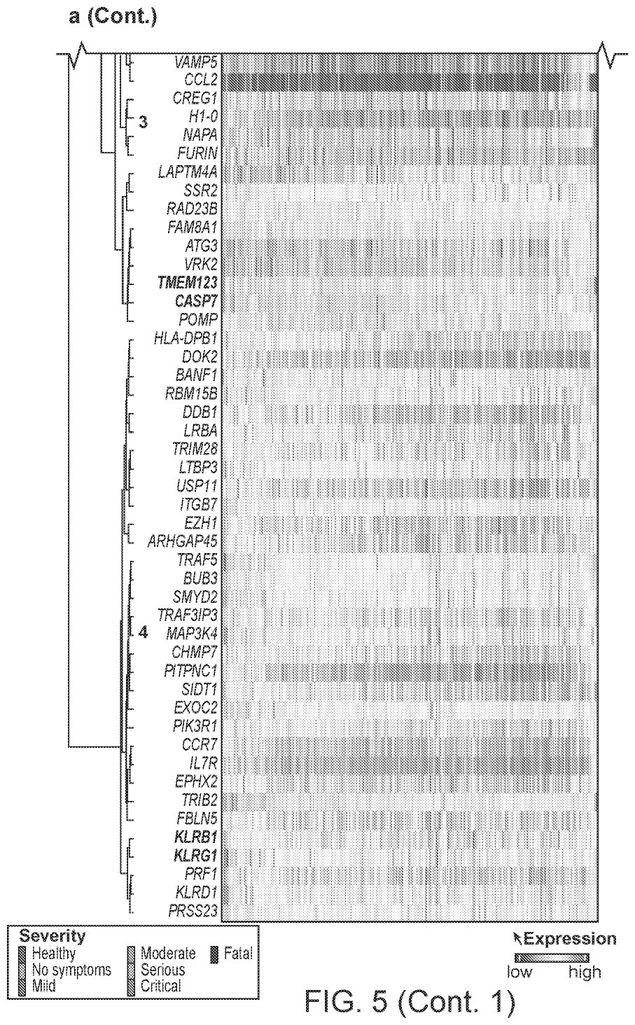

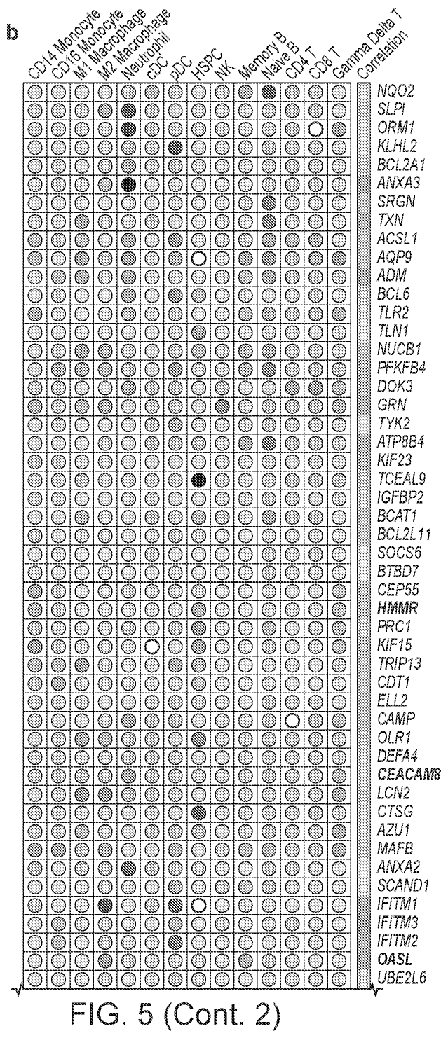

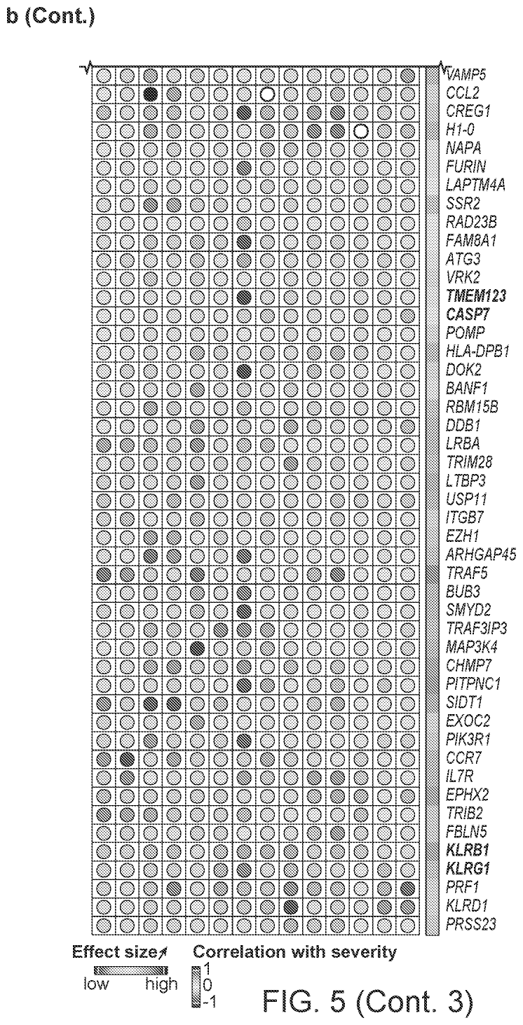

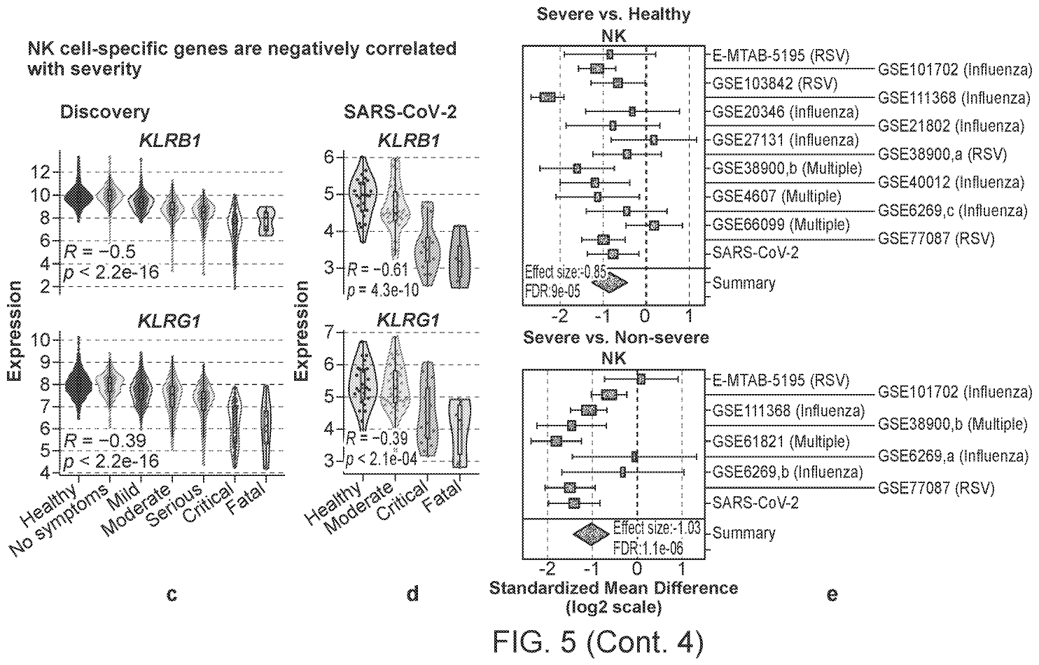

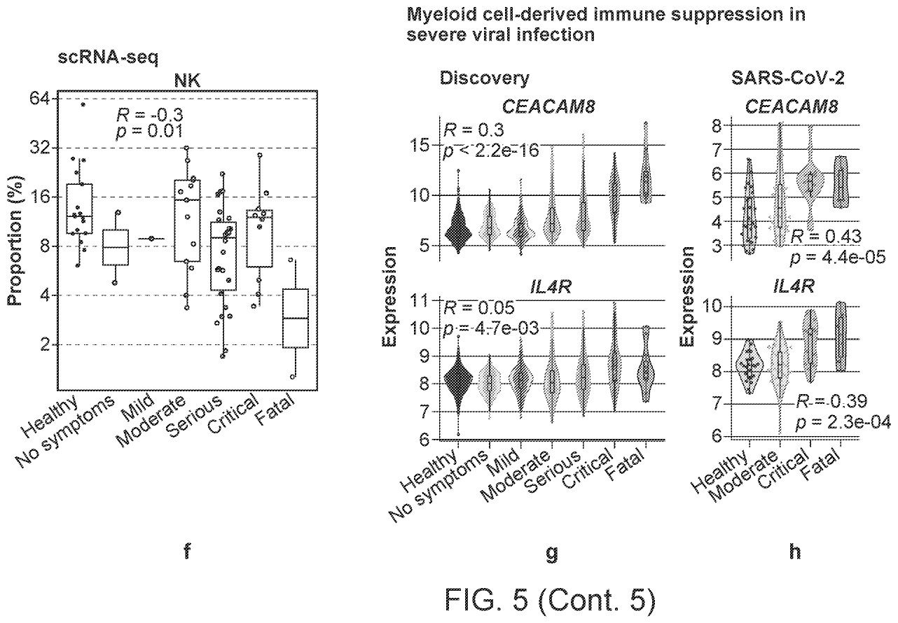

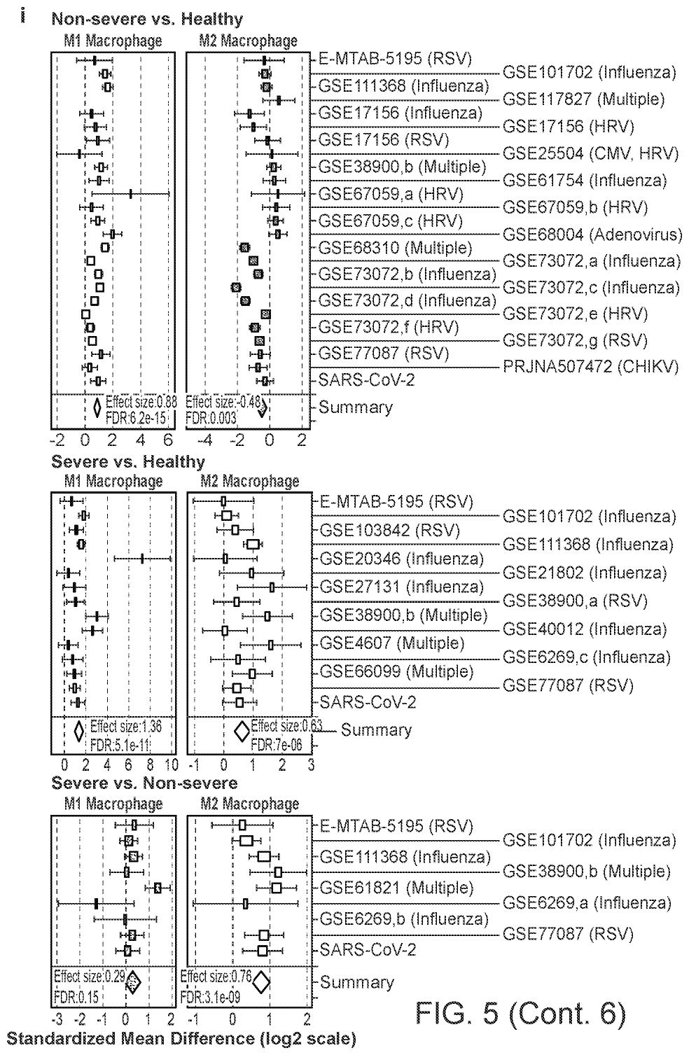

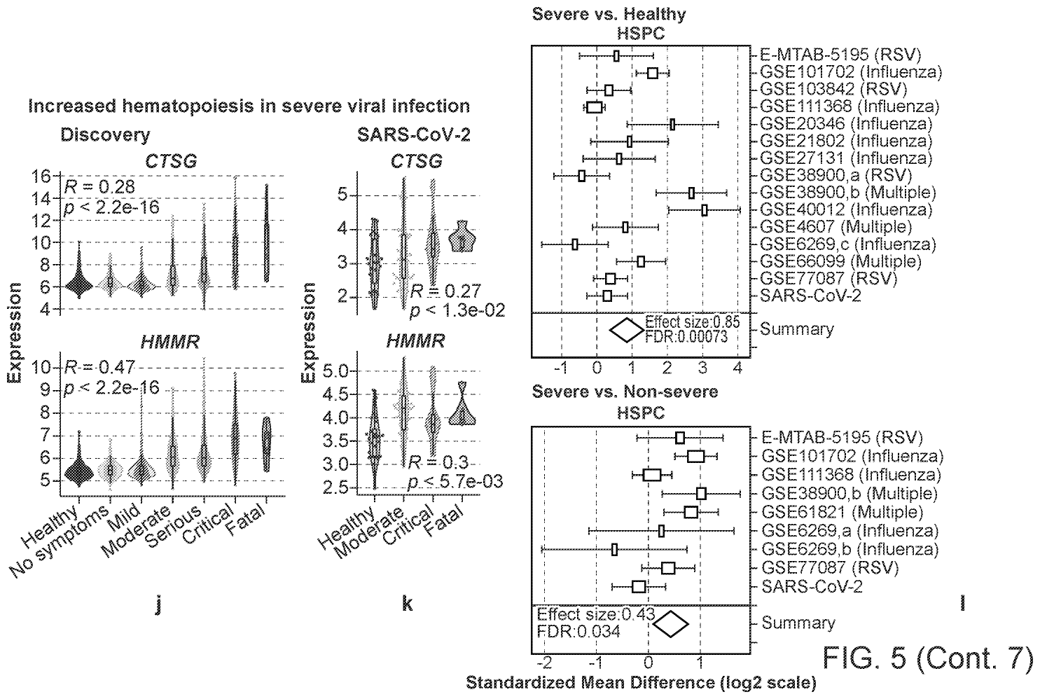

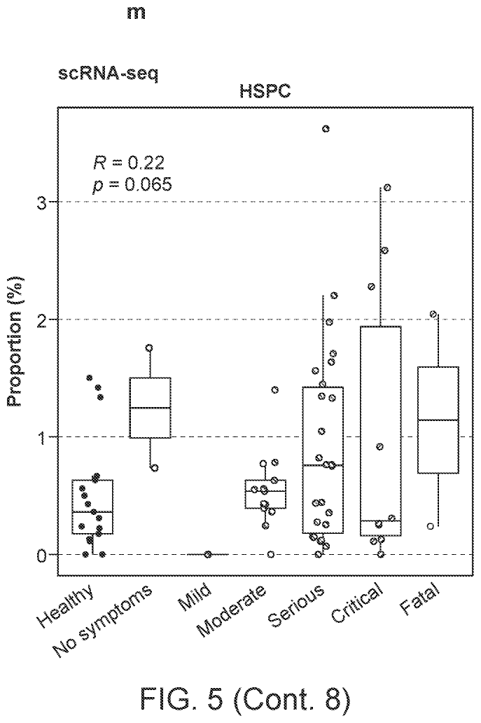

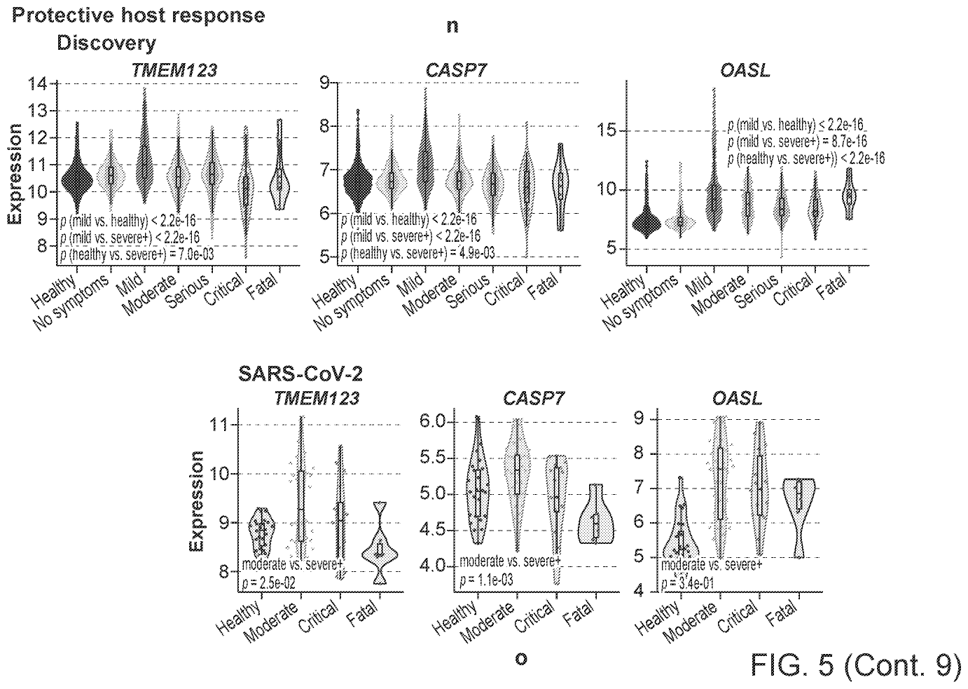

96 genes was identified within the MVS that were significantly different between the two trajectories (FIG. 5A). The majority of the genes negatively correlated with the severity of viral infection are preferentially expressed in lymphocytes (T cells, B cells and NK cells), whereas the majority of the genes positively correlated with severity are preferentially expressed in myeloid cells (granulocytes, monocytes, mDCs, and macrophages). Expression of each gene in a cell type was obtained, relative to all other cell types, from the MetaSignature database (FIG. 5B) (Haynes et al., 2017; Vallania et al., 2018).

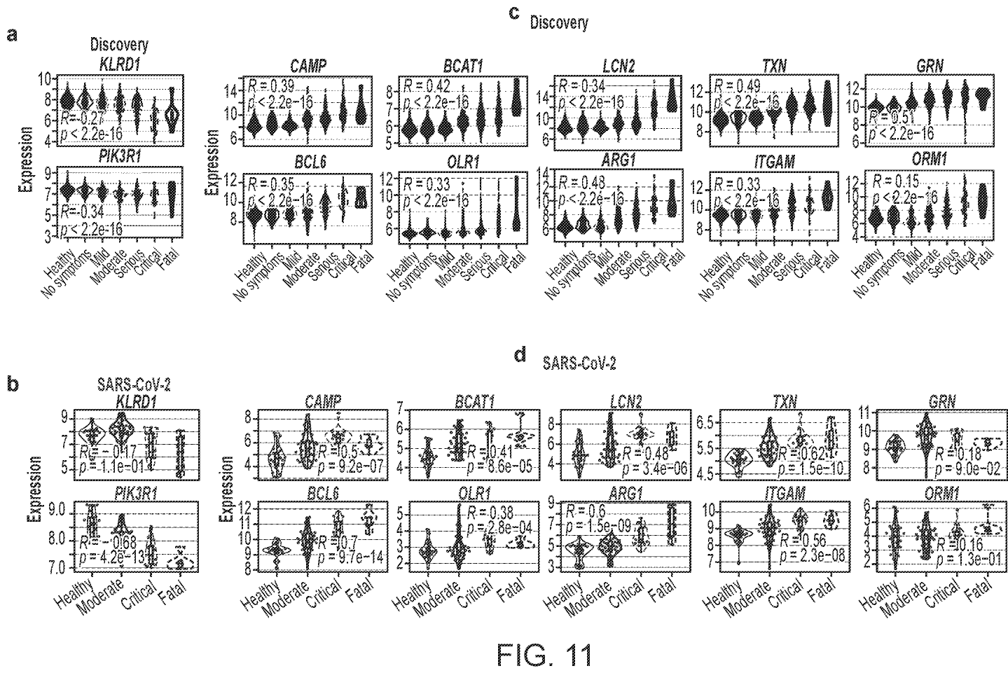

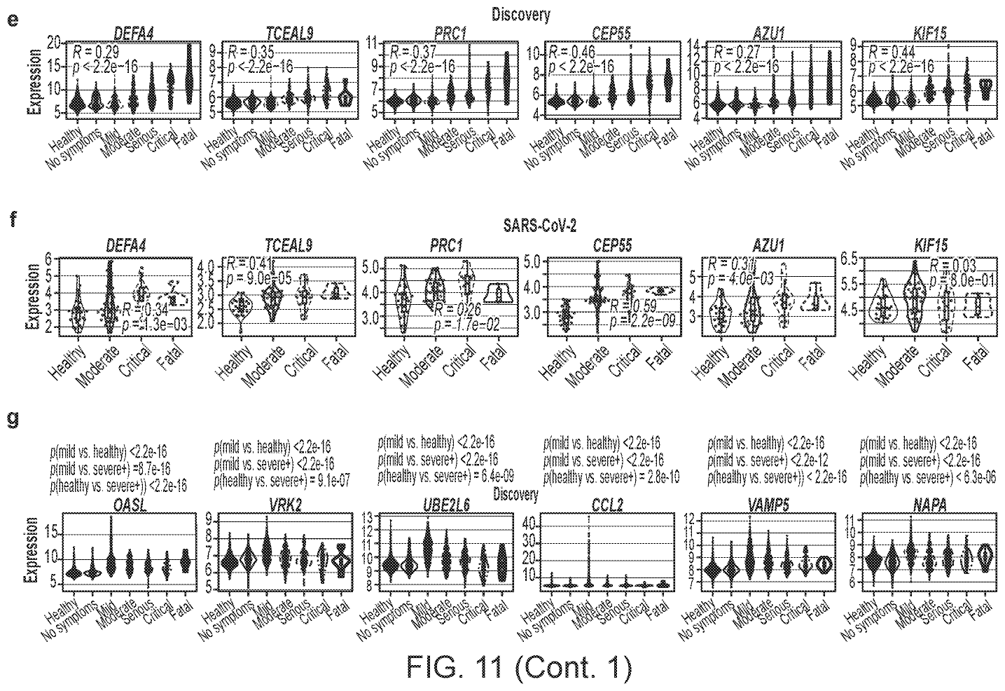

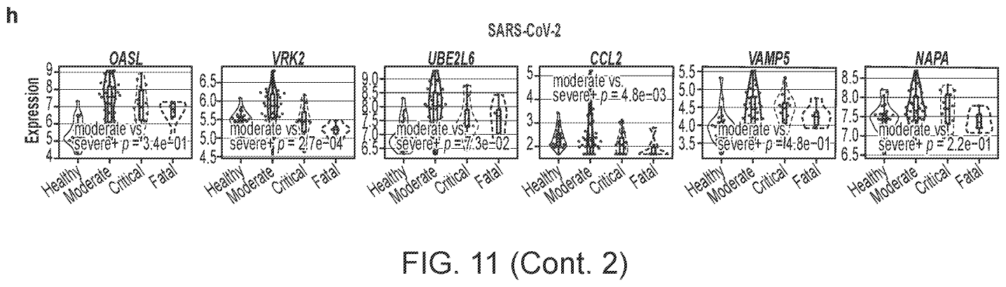

Trajectory analysis identified several NK cell-specific genes from the killer cell lectin-like receptor (KLR) family (KLRB1, KLRG1, KLRD1) and phosphoinositide-3-Kinase (P13K) signaling genes (PIK3R1), which negatively correlated with severity (FIG. 5C and Figure S4A). P13K signaling in NK cells and mutations in PIK3R1 have been linked with human immunodeficiency and viral infections (Mace, 2018). These genes also significantly decreased in critical and fatal SARS-CoV-2 infections compared to healthy controls (FIG. 5D and FIG. 11B). Therefore, it was hypothesized that NK cell proportions decreased with increased severity of viral infection. In silico cellular deconvolution analysis of bulk transcriptome profiles using immunoStates showed the proportions of NK cells were significantly lower in patients with severe viral infections compared to healthy controls (ES=−0.85, FDR=8.97e-05) and non-severe viral infections (ES=−1.03, FDR=1.13e-06) (FIG. 5E). Further, across the three independent cohorts profiled using scRNA-seq, NK cell proportions in severe patients were lower than in healthy controls (FIG. 5F). Collectively, trajectory analysis using dSpace, deconvolution using immunoStates, and scRNA-seq found that the proportions of NK cells and the expression of several NK cell-associated genes reduced with increased severity of viral infection, irrespective of the infecting virus.

Myeloid-Derived Immune Suppression is Higher in Patients with Severe Viral Infection

In line with the positive correlation between the MVS score, proportions of myeloid cells, and infection severity, several differentially expressed genes between the two trajectories (FIG. 5A) were preferentially expressed by immune cells of the myeloid lineage (FIG. 5B). As expected, a subset of genes, positively correlated with viral infection severity (CAMP, BCAT1, LCN2, TXN), have known proinflammatory functions in myeloid cells (Bertini et al., 1999; Bruns et al., 2015; Choi and Fujii, 2019; Eriksson et al., 2017; Papathanassiu et al., 2017; Ramos-Martínez et al., 2018) (FIG. 11C).

However, strong evidence of increased myeloid cell-derived immune suppression in patients with severe viral infection was also found. First, markers of polymorphonuclear myeloid-derived suppressor cells (PMN-MDSCs), CEACAM8 (CD66B; FIG. 5G) and OLR1 (LOX-1; FIG. 11C), were higher in patients with severe viral infection. Second, markers of monocytic MDSCs (M-MDSCs), IL-4R (FIG. 5G), ITGAM(CD11B; FIG. 11D), and a functional marker of MDSCs, ARG1 (FIG. 11D), were also positively correlated with the severity of viral infection. Third, ORM1, which drives the differentiation of monocytes to anti-inflammatory M2b macrophages (Nakamura et al., 2015), was significantly different between the two trajectories. Fourth, genes known to reduce type I interferon response, GRN and BCL6 (Wei et al., 2019; Wu et al., 2016), were positively correlated with severity (FIG. 11C). Most importantly, all genes but GRN positively correlated with severity in the independent cohort of patients with SARS-CoV-2 infection (FIG. 5H and FIG. 11D). Notably, ORM1 expression was significantly lower in non-severe patients but significantly higher in severe patients compared to healthy controls (FIG. 11C). ORM1 expression also showed the same trends in the independent cohort of patients with SARS-CoV-2 infection, although this was not statistically significant (FIG. 11D).

Based on these multiple lines of evidence, it was hypothesized that proportions of pro- and anti-inflammatory macrophages will differ between patients with non-severe versus severe viral infection. To test this hypothesis, we extended our in silico cellular deconvolution analysis using immunoStates. Proportions of pro-inflammatory (M1) macrophages were higher in patients with non-severe (ES=0.88, FDR=6.16e-15) and severe (ES=1.36, FDR=5.12e-11) viral infections compared to healthy controls. In contrast, when compared to healthy controls, proportions of anti-inflammatory (M2) macrophages were lower in non-severe patients (ES=−0.48, FDR=3.00e-03), but higher in severe patients (ES=0.63, FDR=7.02e-06) (FIG. 5I). Proportions of M2 macrophages were also higher in severe patients compared to non-severe patients (ES=0.76, FDR=3.12e-09), but proportions of M1 macrophages were not statistically different (FIG. 5I). Collectively, trajectory analysis and in silico deconvolution provide strong evidence of increased myeloid-derived immune suppression in patients with severe viral infection. Importantly, these results suggest MDSCs as potential therapeutic targets for patients with severe viral infection.

Increased Hematopoiesis in Patients with Severe Viral Infections

Several significantly different genes between the two trajectories (CTSG, PRC1, DEFA4, KIF15, TCEAL9, HMMR, CEP55, and AZU1) were over-expressed in patients with severe viral infection, but not in those with non-severe viral infection compared to healthy controls (FIG. 5J and FIG. 11E). Using the MetaSignature database, we found all but one of these genes (DEFA4) are preferentially expressed at significantly higher levels in circulating hematopoietic stem and progenitor cells (HSPCs) (FIG. 5B). These genes were also over-expressed in patients with severe SARS-CoV-2 viral infection in an independent cohort (FIG. 5K and FIG. 11F). Therefore, it was investigated whether HSPCs were higher in patients with severe viral infection, but not in those with non-severe viral infection. Deconvolution analysis using immunoStates found that HSPCs were significantly higher in patients with severe viral infection compared to healthy controls (ES=0.85, FDR=7.33e-04) and compared to patients with non-severe viral infection (ES=0.43, FDR=3.38e-02) (FIG. 5L), but not in those with non-severe viral infection compared to healthy controls. Finally, in line with the trajectory and deconvolution analyses, the proportions of HSPCs increased with severity in scRNA-seq across three independent cohorts of patients with SARS-CoV-2 (FIG. 5M).

Trajectory Analysis Identifies a Protective Host Response Associated with Mild Viral Infections

Finally, dSpace analysis identified several genes (CCL2, OASL, CASP7, TMEM123, MAFB, VRK2, UBE2L6, NAPA) significantly higher in patients with mild viral infection than those with severe viral infection or healthy controls (FIG. 5N and FIG. 11G). Specifically, it was observed that high expression of CCL2, a type I interferon receptor-mediated chemoattractant, which promotes monocyte migration to the site of infection, and OASL, a type I interferon-induced gene, in patients with mild viral infection. CASP7 is cleaved by CASP3 and CASP10, and is activated upon cell death stimuli and induces apoptosis. TMEM123, also known as PORIMIN, is a cell surface receptor that mediates oncosis, a type of cell death distinct from apoptosis characterized by a loss of cell membrane integrity without DNA fragmentation. These genes were also under-expressed in patients with severe SARS-CoV-2 viral infection compared to those with moderate infection (FIG. 5O, FIG. 11H). Collectively, these results suggest that patients with better ability to recruit monocytes and respond to interferon, and increased cell death are at a lower risk of severe viral infection.

Coordinated Protective and Detrimental Host Response Modules are Associated with the Severity of Viral Infections

Unsupervised hierarchical clustering grouped the 96 genes from dSpace analysis into four distinct modules (FIG. 5A). The modules are shown in the table below.

| | | | | | |
|---|---|---|---|---|---|
| TYK2 | 1 | ADM | 1 | KIF15 | 2 |
| TLN1 | 1 | BTBD7 | 2 | CEP55 | 2 |
| ORM1 | 1 | SOCS6 | 2 | HMMR | 2 |
| KLHL2 | 1 | IGFBP2 | 2 | KIF23 | 2 |
| NQO2 | 1 | AZU1 | 2 | ATP8B4 | 2 |
| TLR2 | 1 | CTSG | 2 | SSR2 | 3 |
| BCL2A1 | 1 | DEFA4 | 2 | LAPTM4A | 3 |
| BCL6 | 1 | CEACAM8 | 2 | RAD23B | 3 |
| SLPI | 1 | OLR1 | 2 | FAM8A1 | 3 |
| ACSL1 | 1 | TCEAL9 | 2 | CASP7 | 3 |
| NUCB1 | 1 | LCN2 | 2 | ANXA2 | 3 |
| SRGN | 1 | PRC1 | 2 | TMEM123 | 3 |
| AQP9 | 1 | TRIP13 | 2 | NAPA | 3 |
| PFKFB4 | 1 | BCL2L11 | 2 | FURIN | 3 |
| DOK3 | 1 | CAMP | 2 | ATG3 | 3 |
| TXN | 1 | ELL2 | 2 | SCAND1 | 3 |
| GRN | 1 | BCAT1 | 2 | VRK2 | 3 |
| ANXA3 | 1 | CDT1 | 2 | CCL2 | 3 |
| UBE2L6 | 3 | EPHX2 | 4 | ARHGAP45 | 4 |
| POMP | 3 | SIDT1 | 4 | RBM15B | 4 |
| MAFB | 3 | LTBP3 | 4 | TRIM28 | 4 |
| CREG1 | 3 | MAP3K4 | 4 | PIK3R1 | 4 |
| H1-0 | 3 | LRBA | 4 | PRSS23 | 4 |
| VAMP5 | 3 | HLA-DPB1 | 4 | FBLN5 | 4 |
| IFITM2 | 3 | DDB1 | 4 | TRIB2 | 4 |
| OASL | 3 | IL7R | 4 | EZH1 | 4 |
| IFITM1 | 3 | CCR7 | 4 | KLRD1 | 4 |
| IFITM3 | 3 | USP11 | 4 | DOK2 | 4 |
| TRAF5 | 4 | BUB3 | 4 | ITGB7 | 4 |
| PITPNC1 | 4 | SMYD2 | 4 | PRF1 | 4 |
| KLRB1 | 4 | TRAF3IP3 | 4 | EXOC2 | 4 |
| CHMP7 | 4 | KLRG1 | 4 | BANF1 | 4 |

Module 1 and 2 were composed of genes preferentially expressed in myeloid and HSPCs, and were higher in patients with severe viral infection (FIG. 5B), whereas module 4 was composed of genes preferentially expressed in lymphoid cells (NK, T, and B cells) and were higher in patients with mild viral infection compared to those with severe infection (FIG. 5B). Module 3 included genes expressed at higher levels in patients with mild viral infection and associated with a protective response. Therefore, these four modules broadly divided the host response genes differentially expressed between two trajectories into two categories: a detrimental host response represented by module 1 and 2 (higher in patients with severe viral infection), and a protective host response represented by module 3 and 4 (higher in patients with mild viral infection). 42 out of 96 genes were selected with significantly high effect size (|effect size|≥1) between the severe and mild trajectories, which included 11, 13, 10, and 8 genes in modules 1, 2, 3, and 4, respectively. These genes and their effect scores are shown below;

| | | | | | |
|---|---|---|---|---|---|
| TXN | 1.65 | DEFA4 | 1.35 | OASL | −1.32 |
| ORM1 | 1.58 | CTSG | 1.31 | TMEM123 | −1.32 |
| PFKFB4 | 1.50 | CEACAM8 | 1.30 | VRK2 | −1.26 |
| SLPI | 1.44 | BCAT1 | 1.26 | NAPA | −1.09 |
| BCL6 | 1.32 | BTBD7 | 1.23 | CCL2 | −1.09 |
| NQO2 | 1.26 | KIF15 | 1.19 | MAFB | −1.09 |
| AQP9 | 1.19 | AZU1 | 1.17 | VAMP5 | −1.07 |
| ANXA3 | 1.07 | CEP55 | 1.16 | ATG3 | −1.01 |
| DOK3 | 1.07 | PRC1 | 1.16 | HLA-DPB1 | −1.82 |
| KLHL2 | 1.01 | HMMR | 1.06 | SMYD2 | −1.39 |
| TYK2 | 1.01 | BCL2L11 | 1.02 | SIDT1 | −1.39 |
| CAMP | 1.54 | UBE2L6 | −1.84 | TRIB2 | −1.35 |
| LCN2 | 1.40 | CASP7 | −1.44 | DOK2 | −1.24 |
| KLRB1 | −1.13 | EXOC2 | −1.11 | BUB3 | −1.11 |

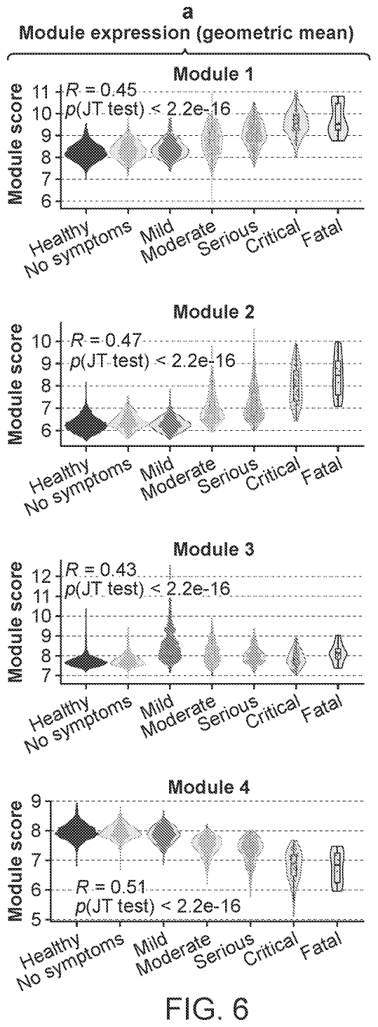

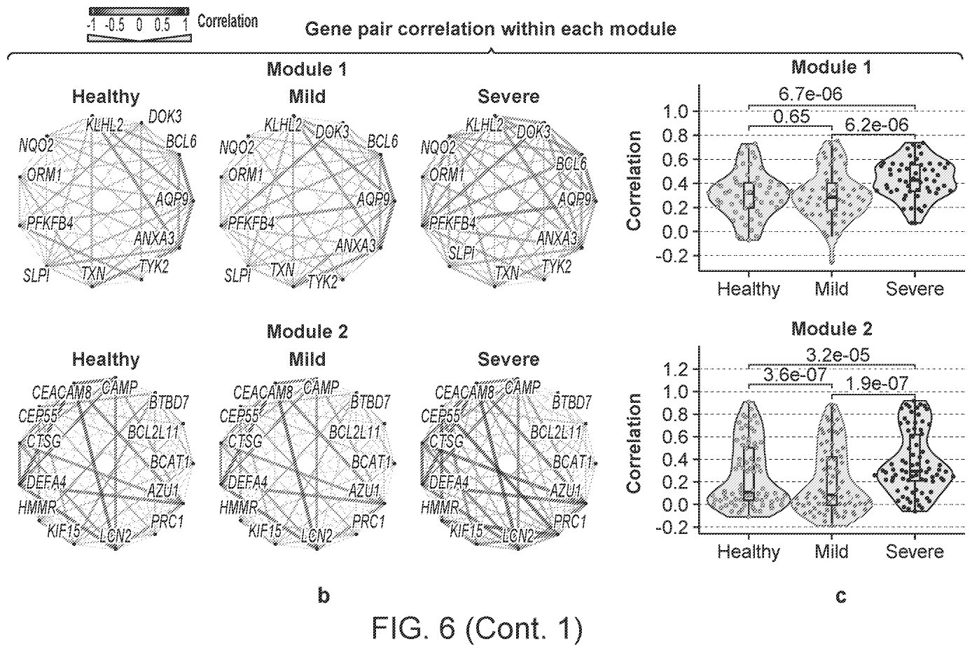

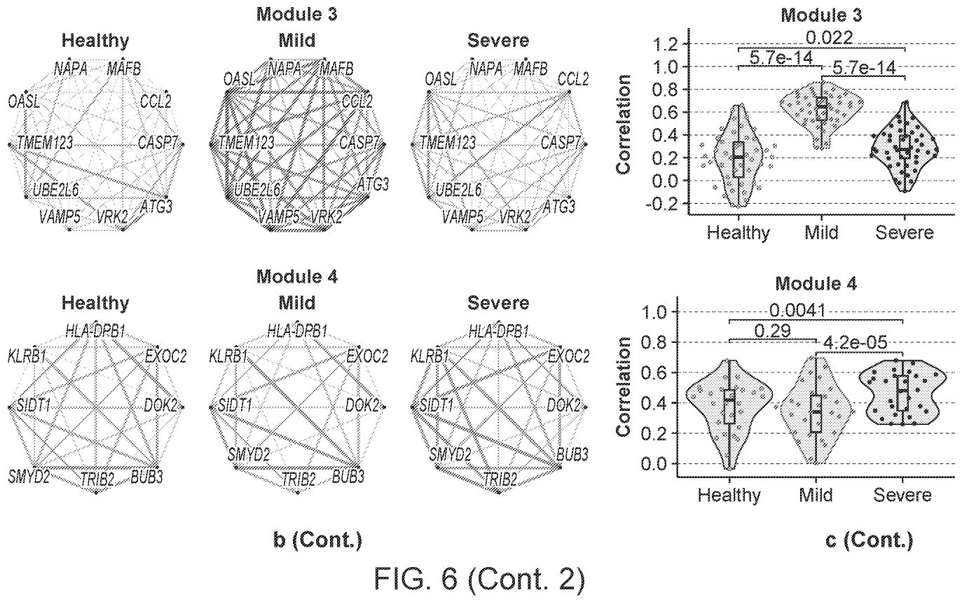

Module scores, defined as the geometric mean of expression of genes in a given module, using these reduced sets of genes continued to be significantly positively (module 1, 2, and 3) and negatively (module 4) correlated with severity of viral infection (|r|≥0.43, p<2.23-16; FIG. 6A), which further suggested that genes within each module are correlated with each other. Indeed, we found most pairs of genes within each module were positively correlated, irrespective of their infection status (FIG. 6B).

Interestingly, it was found the correlation structure within each module changed depending on the presence and severity of infection. The distribution of pairwise correlations between genes in modules 1, 2, and 4 was significantly higher in patients with severe viral infection than healthy controls or patients with mild viral infection (p<5e-05; FIG. 6C). Interestingly, the distribution of pairwise correlations in module 2 was significantly lower in patients with mild infection compared to healthy controls (p=3.6e-07; FIG. 6C). In contrast, pairwise correlations between genes in module 3, which included genes involved in the protective host response, were significantly higher in patients with mild infection compared to healthy controls and those with severe infection (p=5.7e-14; FIG. 6C). Collectively, these results demonstrate that the genes within each module are expressed in a coordinated manner depending on the infection status and severity of infection.

The Protective Host Response Module is Decoupled from the Interferon Response in Patients with Severe Viral Infection

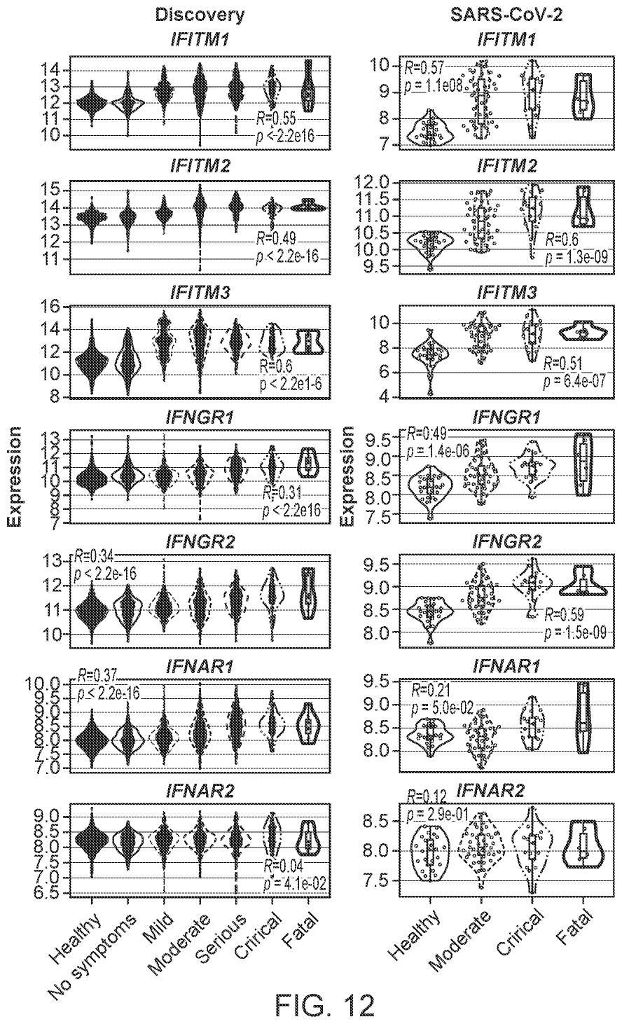

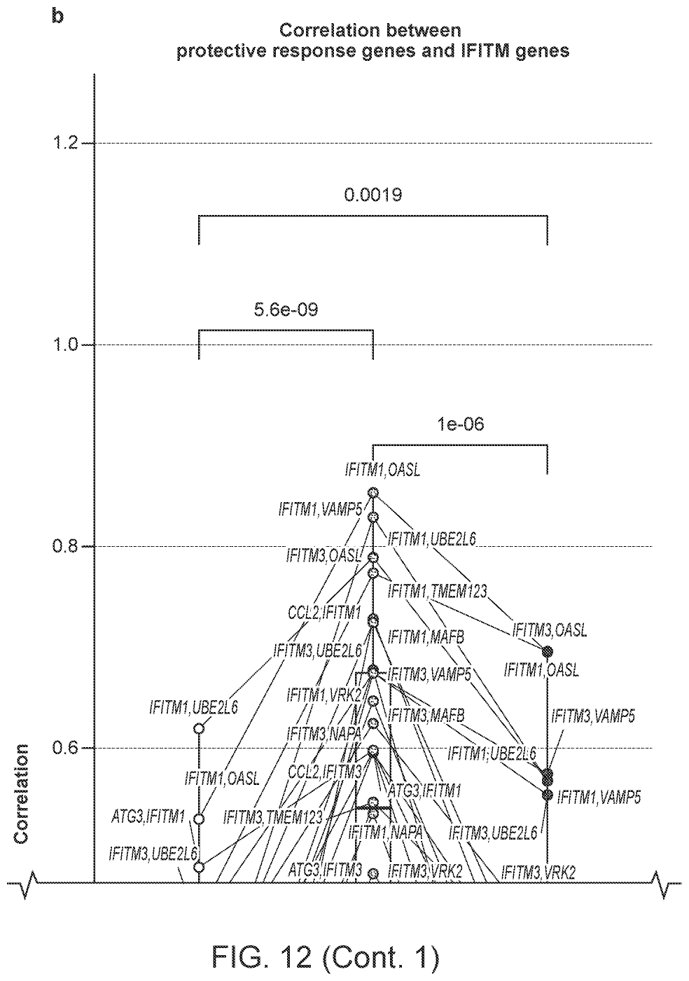

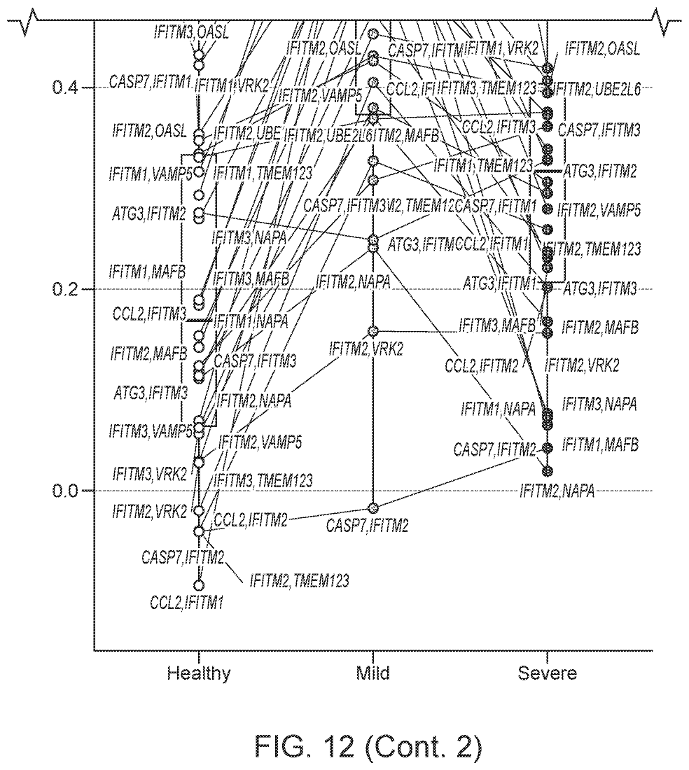

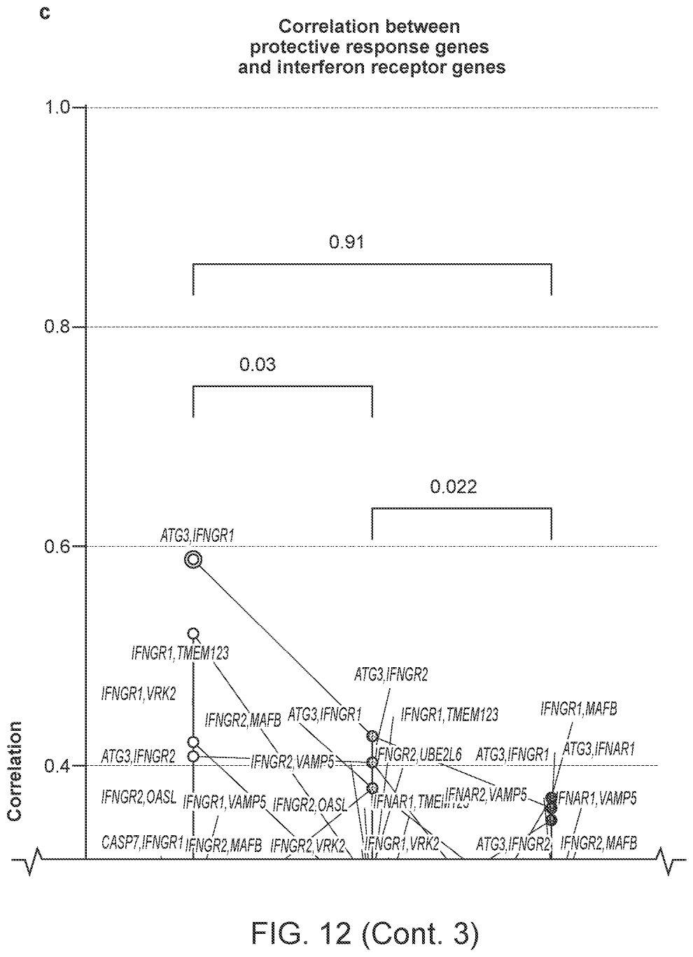

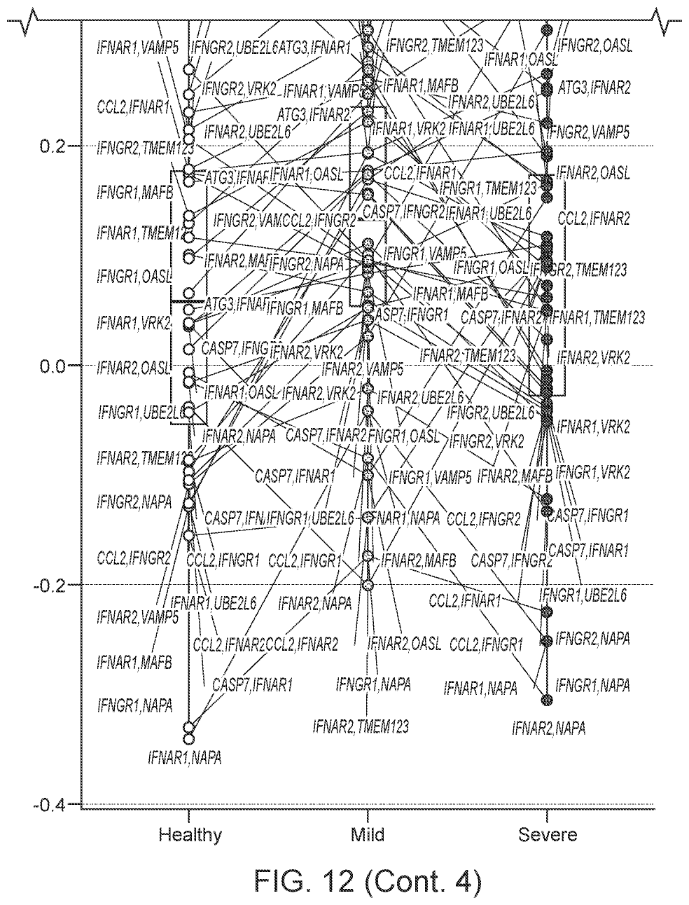

Recent reports have described higher expression of interferon-stimulated genes (ISGs) in patients with moderate SARS-CoV-2 infection than those with severe infection (Arunachalam et al., 2020). Therefore, it was investigated whether this observation is generalizable to other viruses. Indeed, module 3 included three interferon-induced transmembrane (IFITM) genes (IFITM1, IFITM2, IFITM3), involved in the restriction of multiple viruses (Bailey et al., 2014), that were over-expressed in patients with viral infections and positively correlated with severity (FIG. 12A). It was also found several type I and type II interferon receptors over-expressed during viral infection that positively correlated with severity, irrespective of the infecting virus (FIG. 12A). Strikingly, in patients with mild viral infection, the distribution of correlations between IFITMs and genes in the protective response module 3 was significantly higher than in patients with severe viral infection or healthy controls (p<1e-06; FIG. 12B). Further, the distribution of correlations between the type I and II interferon receptors and the protective response module 3 was not statistically different between healthy controls and patients with severe viral infection, but was significantly higher in patients with mild viral infection (FIG. 12C). These results show that while expression of the ISGs increase with the severity of viral infection, their correlation with the protective host response does not increase in patients with severe viral infection as much as those with mild viral infection. Collectively, these results demonstrate decoupling of the protective host response from the interferon response in patients with severe viral infection, irrespective of the virus.

Host Response-Based Module Score Improves Classification of Patients with Severe and Non-Severe Viral Infections

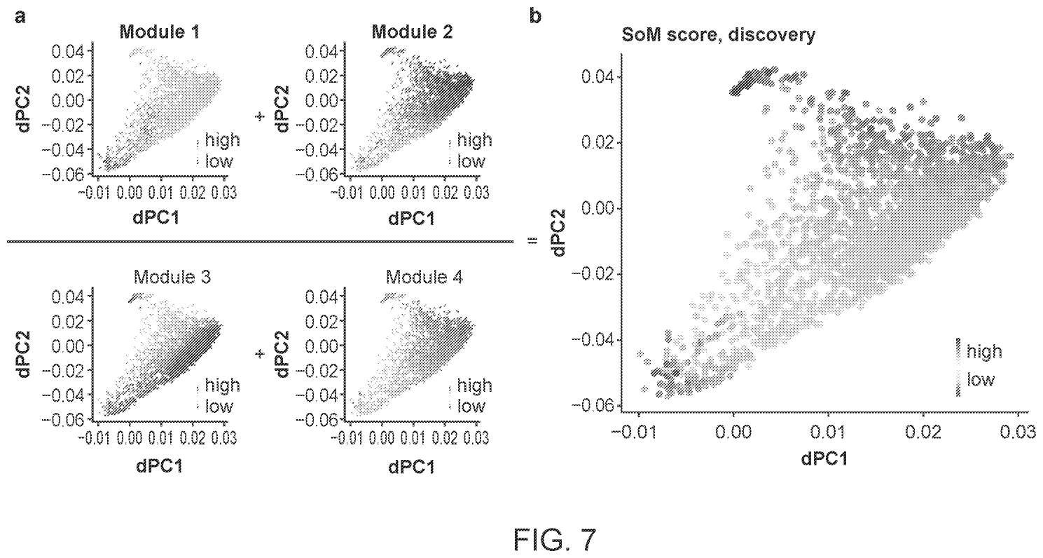

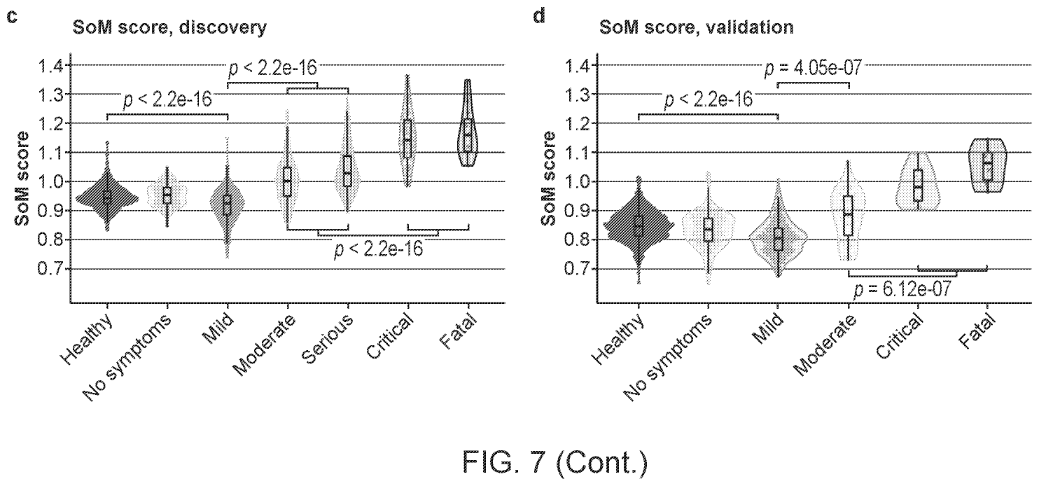

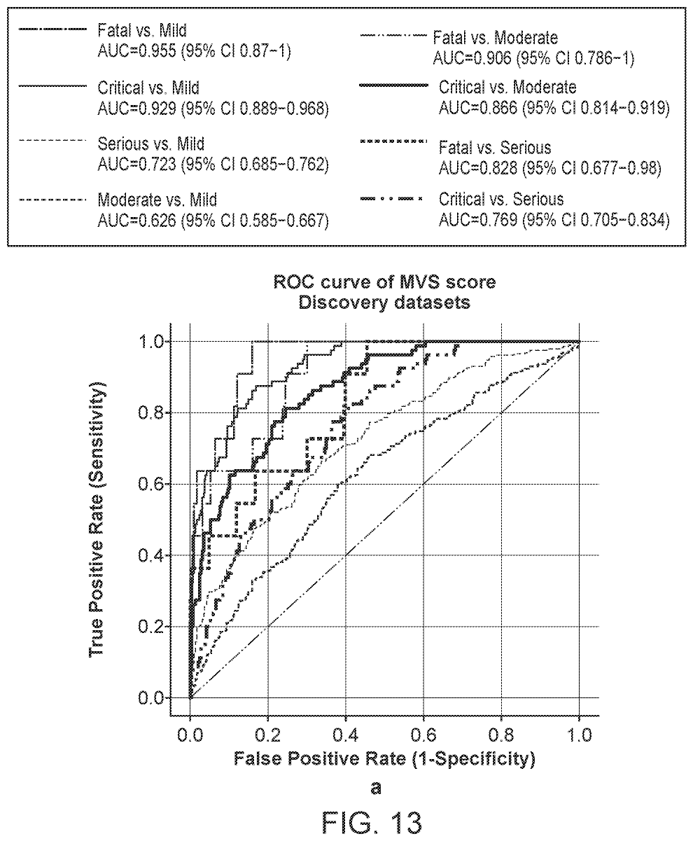

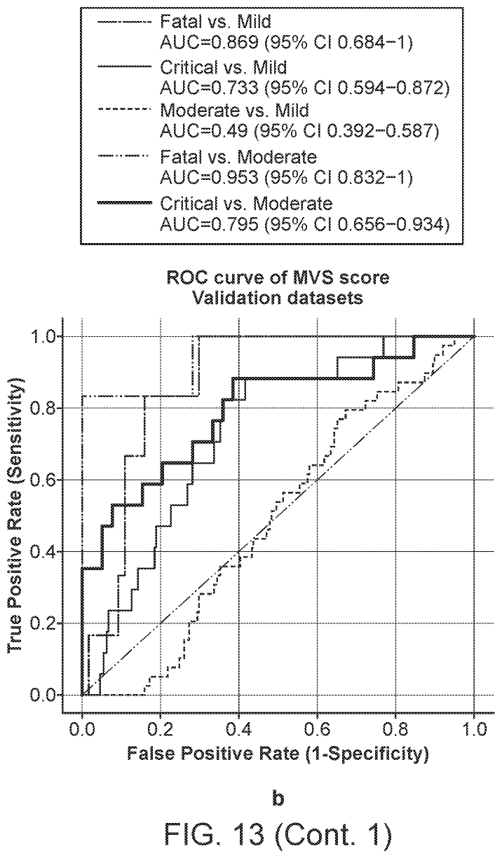

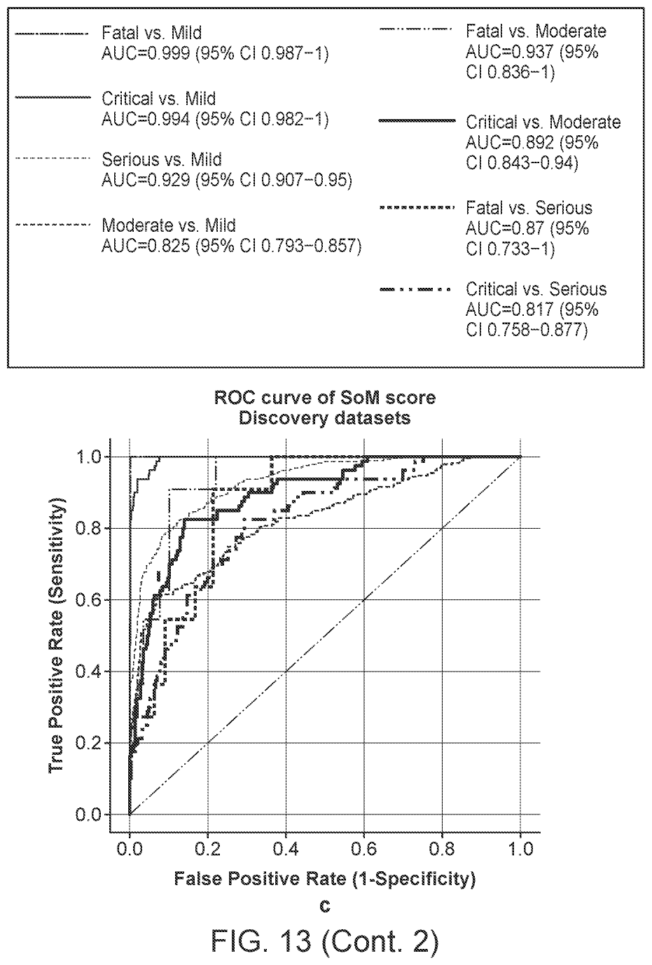

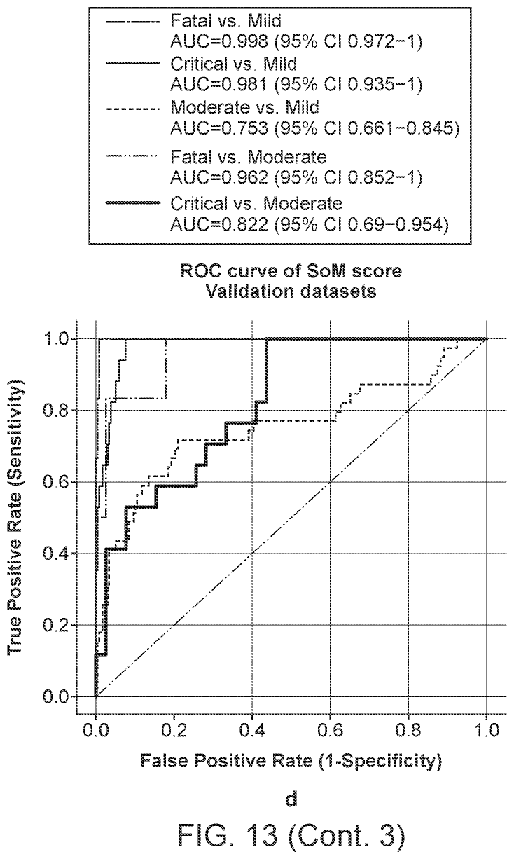

Despite correlating significantly with the severity of viral infection, the MVS score is unable to separate severe from non-severe patients with clinically relevant accuracy (FIG. 13A-B). It was hypothesized that a score that considers the distinction between the protective and detrimental host responses would improve discrimination between patients with severe and non-severe viral infection. The two deleterious host response module scores (modules 1 and 2) were higher in samples along the severe trajectory than those along the mild trajectory, whereas the two protective host response module scores (modules 3 and 4) had the opposite pattern of expression (FIG. 7A). The Severe-or-Mild (SoM) score of a sample was defined as the sum of the scores for module 1 and 2 divided by the sum of the scores for module 3 and 4 (Methods). The SoM score showed a more pronounced gradient between the severe and mild trajectories than any of the individual module scores (FIG. 7B). Indeed, across the 3,183 samples used for discovery of the trajectories, the SoM score distinguished patients with mild infection from those with severe infection with AUROC 0.929 (FIG. 7C, FIG. 13C). Importantly, the SoM score also distinguished patients with mild infection from those with severe infection with very high accuracy (AUROC>0.98) in 5 independent validation cohorts comprised of 1,154 samples from patients infected with 4 different viruses (SARS-CoV-2, influenza, HRV, chikungunya) (FIG. 7D, FIG. 13D). Collectively, these results demonstrate that the protective and detrimental host response modules identified by trajectory analysis improve discrimination accuracy between patients with mild and severe viral infection. These results further suggest that suppressing the detrimental host response modules or enhancing the protective host response modules could be therapeutic targets for host-directed broad-spectrum intervention in patients with severe viral infections.

The four pandemic viral outbreaks between 2009 and 2019 have underscored an urgent unmet need for identifying generalizable diagnostic and prognostic tests. Such tests could be readily deployed for triage by identifying patients at higher risk of severe outcome and avoid overwhelming healthcare systems worldwide that could wreak havoc with extremely high socioeconomic costs as the SARS-CoV-2 pandemic has shown.

The present study is believed to be the largest, most comprehensive systems immunology analysis of blood transcriptome profiles from patients with viral infections to date by integrating 4,780 blood transcriptome profiles from patients with one of 16 viral infections across 34 independent cohorts from 18 countries. Further, scRNA-seq profiles of 264,000 immune cells from 71 samples across 3 independent cohorts were integrated with blood transcriptome profiles. this analysis leveraged the biological, clinical, and technical heterogeneity across these 37 cohorts to demonstrate that a conserved host response to viral infection is (1) associated with severity, (2) predominantly driven by myeloid cells, and (3) defines distinct trajectories for mild or severe outcomes in patients with viral infection. Using these trajectories, it was shown that increased hematopoiesis, myelopoiesis and myeloid-derived suppressor cells, and reduced NK and T cells are associated with increased severity across all viral infections. Importantly, trajectory analysis identified four gene modules, two of which are associated with a detrimental response leading to a severe outcome, and the other two with a protective response leading to mild infection. Finally, the SoM score was defined using these modules that accurately distinguish patients with mild or moderate viral infections from those with severe outcomes.

This analysis provides strong evidence of a conserved host immune response to several viruses that is associated with severity of viral infection. Although the MVS was identified by analyzing three respiratory viruses (influenza, RSV, and HRV), it is generalizable across novel viruses, including SARS-CoV-2, chikungunya, and Ebola. The results also demonstrate that the conserved host response to viral infections is generalizable across ages. Out of 37 cohorts, 12 cohorts consisted of 931 samples from children (<18 years), most of which (643 samples in 6 cohorts) were children younger than 2 years. While majority of the research on the host immune response to SARS-CoV-2 during the ongoing pandemic has focused on understanding its dysregulation, how it differs from other viruses, and its association with severity, this conserved similarity in dysregulation of the host immune response in patients with severe outcome, irrespective of the virus, presents several opportunities for global pandemic preparedness for developing novel diagnostic and prognostic tests, identifying novel drug targets for host-directed broad-spectrum anti-viral therapies, and drug repurposing for the pandemics that will invariably come in the future.

Unlike the MVS score, the SoM score distinguished patients with a severe outcome from those with a non-severe outcome with very high accuracy. This clinically meaningful increase in accuracy for the SoM score is due to the four gene modules that are associated with either a detrimental or protective host response to viral infection. In contrast, the MVS score considers all genes equal irrespective of their protective or detrimental role. Such a conserved gene signature, identified using a large amount of heterogeneous data across multiple cohorts, can be further analyzed to identify a parsimonious, clinically useful, point-of-care test that is generalizable across patient populations. For example, a 3-gene host response-based signature for diagnosis of tuberculosis that has been shown to be generalizable across a large number of patient populations, and has been translated into a proof-of-concept point-of-care cartridge with high accuracy. In addition, given the high pairwise correlation between genes within each module, only a small subset of genes within each module would provide the same discriminatory power, further allowing selection of a parsimonious gene signature. Importantly, trajectory analysis suggests that the SoM score has the potential to predict severity of outcome in patients with viral infection, though it needs to be tested in additional cohorts. Such a test would be an indispensable tool to avoid overwhelming the healthcare system during a viral outbreak by identifying patients who can safely recover at home and those who should be admitted to a hospital. Importantly, this test could also be used for identifying patients at high risk of severe outcomes in clinical practice outside of outbreaks.

One of the four modules, module 3, included a monocyte chemoattractant (CCL2), a regulator of type I interferon transcription (MAFB), interferon-induced genes (ISGs; OASL, UBE2L6), and genes involved in cell death (TMEM123, CASP7). These genes were over-expressed in patients with mild viral infection compared to healthy controls and those with severe viral infection, and highly correlated with each other in patients with mild viral infection, but not in those with severe viral infection, irrespective of virus, suggesting highly coordinated immune response between monocyte recruitment, interferon response and cell death is associated with protection. The results are consistent with recent observations that ISGs are strongly induced in patients with moderate SARS-CoV-2 infection compared to those with severe SARS-CoV-2 infection (Arunachalam et al., 2020; Hadjadj et al., 2020), and generalize to patients with non-severe viral infections compared to those with severe infection, irrespective of the virus.

Importantly, the genes in module 3 were more correlated with multiple interferon-induced transmembrane proteins (IFITMs) in patients with mild infection compared to those with severe viral infection. IFITMs are involved in restricting viruses at various stages of the life cycle including (1) blocking host cell entry by trapping virions in endosomal vesicles, (2) inhibiting viral gene expression and protein synthesis, and (3) disrupting viral assembly (Liao et al., 2019; Zhao et al., 2018). The lower correlations between the expression of the IFITM genes and the genes in module 3 strongly suggest that in patients with severe viral infection, the interferon-induced response is “decoupled” from the protective response. Understanding the mechanisms underlying this decoupling could lead to targets for host-directed therapy for viral infection.

Analysis of scRNA-seq in 3 independent cohorts and in silico cellular deconvolution across 32 cohorts found increased HSPCs in patients with severe viral infections, irrespective of the virus. In contrast, it has previously shown reduced proportions of HSPCs in mild viral infections (Bongen Genome Med 2018 PMID: 29898768), which may reflect the production of myeloid cells at the expense of the lymphoid compartment to replenish myeloid cells during infection (Takizawa et al., 2012). Indeed, increased myeloid cells and reduced lymphoid cells have been observed in both scRNA-seq and in silico cellular deconvolution analysis. This result further supports a model where human HSPCs take an active role in the immune response by differentiating into myeloid cells, similar to what we have previously observed (Bongen et al., 2018). Increased HSPCs proportions in patients with severe viral infection suggests emergency hematopoiesis that is associated with increased risk of severity, irrespective of the virus, as the host immune response fails to adequately respond to the infecting virus.

The MVS is predominantly expressed in the myeloid cells. First, the MVS increased at a single-cell level in CD14+ monocytes, which increase with severity of viral infection, whereas CD16+ monocytes decrease, which is in line with several recent studies of SARS-CoV-2 infected patients (Gatti et al., 2020; Hadjadj et al., 2020; Silvin et al., 2020; Zhou et al., 2020). This suggests that reduced CD16+ monocytes in peripheral blood, possibly due to efflux to the site of infection in response to ongoing tissue damage or dysregulated cytokine sensing, is a conserved feature of the host response in severe viral infections across viruses, and may have prognostic significance. In addition, increased proportions of PMN- and monocytic-MDSCs, and anti-inflammatory macrophages along with higher expression of their phenotypic and functional markers in patients with severe viral infections, irrespective of the virus. Interestingly, in patients with mild viral infection, markers of MDSCs did not increase substantially, and proportions of anti-inflammatory macrophages decreased. These results suggest that lower myeloid-derived suppression in the early phase of infection is protective. These results provide strong evidence that, although increased PMN- and M-MDSCs may limit hyperinflammation as the viral infection continues, they lead to a detrimental amplification of immunosuppression, irrespective of the virus.

Among their immunosuppressive roles, MDSCs are known to suppress NK cell activity through arginase and ROS/RNS (Schrijver et al., 2019). Indeed, the trajectory and in silico deconvolution analyses and scRNA-seq data found several NK cell-specific genes (KLRB1, KLRG1, KLRD1, PIK3R1) are negatively correlated with the severity of viral infection, and proportions of NK cells reduced in patients with severe viral infections. It has been previously shown that healthy individuals with lower expression of KLRD1 are more likely to be infected when challenged (Bongen et al., 2018). A negative correlation between expression of KLRD1 and the severity of viral infections, including SARS-CoV-2, further emphasizes that KLRD1-expressing NK cells may play a protective role in infection upon exposure and severity, irrespective of the infecting virus.

Taken together, these analyses offer a systems view of the immune state during viral infection and factors that mediate and predict progression to mild or severe outcomes, irrespective of the clinical, biological, and technical heterogeneity and the infecting virus. Our findings identified host response modules that could lead to new intervention strategies, including diagnostics for predicting patients at higher risk of severe outcomes, and broad-spectrum host-directed therapies for pandemic preparedness.

### Methods

### Dataset Collection and Preprocessing

26 gene expression datasets were downloaded from the National Center for Biotechnology Information (NCBI) Gene Expression Omnibus (GEO), Sequence Read Archive (SRA), ArrayExpress, and European Nucleotide Archive (ENA) consisting of 4,780 samples across 34 independent cohorts derived from whole blood or peripheral blood mononuclear cells (PBMCs). The samples in these datasets represented the biological and clinical heterogeneity observed in the real-world patient population, including healthy controls and patients infected with 16 different viruses with severity ranging from asymptomatic to fatal viral infection over a broad age range (<12 months to 73 years) (FIG. 1A). Notably, the samples were from patients enrolled across 18 different countries representing diverse genetic backgrounds of patients and viruses. The technical heterogeneity in the analysis were included as these datasets were profiled using microarray and RNA sequencing (RNA-seq) from different manufacturers.

All microarray datasets were renormalized using standard methods when raw data were available from the GEO database. GC robust multiarray average (gcRMA) were applied to arrays with mismatch probes for Affymetrix arrays. Normal-exponential background correction followed by quantile normalization for Illumina, Agilent, GE, and other commercial arrays was used. Custom arrays and used preprocessed data as made publicly available by the study authors were not renormalized. Microarray probes was mapped in each dataset to Entrez Gene identifiers (IDs) to facilitate integrated analysis. If a probe matched more than one gene, the expression data was expanded for that probe to add one record for each gene. When multiple probes mapped to the same gene within a dataset, a fixed-effect model was applied. Within a dataset, cohorts assayed with different microarray types were treated as independent.

### Standardized Severity Assignment

For each dataset, the sample phenotypes were used as defined in the original publication. A severity category was manually assigned to each sample based on the cohort description for each dataset in the original publication as follows: (1) healthy controls —asymptomatic, uninfected healthy individuals, (2) asymptomatic or convalescents—afebrile asymptomatic individuals who tested positive for a virus or those fully recovered from a viral infection with completely resolved symptoms, (3) mild—symptomatic individuals with viral infection that were either managed as outpatient or discharged from the emergency department (ED), (4) moderate—symptomatic individuals with viral infection who were admitted to the general wards and did not require supplemental oxygen, (5) serious —symptomatic individuals with viral infection who were described as ‘severe’ by original authors, admitted to general wards with supplemental oxygen, or admitted to the intensive care unit (ICU) without requiring mechanical ventilation or inotropic support, (6) critical —symptomatic individuals with viral infection who were on mechanical ventilation in the ICU or were diagnosed with acute respiratory distress syndrome (ARDS), septic shock, or multiorgan dysfunction syndrome (MODS), and (7) fatal—patients with viral infection who died in the ICU.

For datasets that did not provide sample-level severity data (GSE101702, GSE38900, GSE103842, GSE66099, GSE77087), severity categories were assigned as follows. All samples in a dataset were categorized as “moderate” when either (1) >70% of patients were admitted to the general wards as opposed to discharged from the ED, (2) <20% of patients admitted to the general wards required supplemental oxygen, or (3) patients were admitted to the general wards and categorized as ‘mild’ or ‘moderate’ by the original authors. All samples were in a dataset categorized as “severe” when >20% of patients had either (1) been admitted to the general wards and categorized as ‘severe’ by original authors, (2) required supplemental oxygen, or (3) required ICU admission without mechanical ventilation.

### Viral Challenge Studies

GSE73072 included seven viral challenge studies that determined the infection status of a subject through reverse transcription PCR (RT-PCR) for a given virus (H1N1, H3N2, RSV, HRV) in longitudinally collected nasopharyngeal samples. In these studies, we assigned all baseline pre-challenge samples and subjects who never shed virus, as determined by RT-PCR, to the ‘healthy’ category. Samples from infected subjects, defined as those who had virus detected in any of their nasopharyngeal samples, were assigned to one of three categories: (1) before infection—blood samples collected after challenge but before a virus was detected in a nasopharyngeal sample, (2) after infection—blood samples collected after the last nasopharyngeal sample in which a virus was detected, and (3) during infection—blood samples collected between the first and last nasopharyngeal sample in which a virus was detected.

### COCONUT Co-Normalization

Combat CONormalization Using conTrols (COCONUT) was assigned for between-dataset normalization (Sweeney et al., 2016b). COCONUT allows for co-normalization of gene expression data without bias towards sample diagnosis by applying a modified version of the ComBat empirical Bayes normalization method (Johnson et al., 2006), which assumes a similar distribution between control samples. Briefly, healthy controls from each cohort undergo ComBat co-normalization without covariates, and the ComBat estimated parameters are computed for healthy samples in each dataset. By applying these parameters to the non-healthy samples, all datasets keep the same background distribution while retaining the same relative distance between healthy and disease samples, which preserves the biological variability between the two groups within a dataset. It has been previously shown that when COCONUT co-normalization is applied, housekeeping genes remain invariant across both conditions and cohorts, and each gene retains the same distribution across conditions within each dataset (Sweeney et al., 2016b).

### MVS Genes and Score

A de novo gene signature was not derived to represent a conserved host response to viral infections. Instead, a previously described 396-gene signature from peripheral blood (Andres-Terre et al., 2015) was used. Further, as previously described, the MVS score of a sample was defined as the difference between the geometric mean of the over-expressed genes and the geometric mean of the under-expressed genes in the MVS (Andres-Terre et al., 2015). Out of 396 genes in the MVS, 251 genes (111 over- and 140 under-expressed) were measured across all datasets. Across 4 independent datasets that measured all 396 genes in patients infected with SARS-CoV-2, Ebola, or chikungunya, we found that the MVS score using the 251-gene signature was highly correlated with the MVS score using the 396-gene signature (0.976≤r≤0.997). Thus, the 251-gene signature provided the same information and did not skew our results. We measured the correlation of the MVS score with viral infection severity using Spearman's rank correlation coefficient. The Mann-Whitney U test (Wilcoxon rank-sum test) was used to compare MVS scores between two groups. The trend of the MVS score along viral infection severity categories was tested using the Jonckheere-Terpstra trend test.

### RNA Sequencing Analysis

The raw reads for the Ebola (PRJNA352396) and chikungunya (PRJNA507472 and PRJNA390289) cohorts were obtained from from the European Nucleotide Archive (ENA). The RNA-seq raw reads of the SARS-CoV-2 cohort were obtained from Inflammatix. The quality of the raw reads was assessed with Trim Galore (v0.6.5), trimmed Illumina adaptors, and removed reads that were too short after adaptor trimming (less than 20 nt). The cleaned reads were mapped to human genome sequences (hg38) using STAR (v2.7.3) (Dobin et al., 2013). More quality control was performed by checking the quality of the mapped reads in BAM format with Qualimap (v.2.2.2) (Garcia-Alcalde et al., 2012). To quantify gene expression, human transcriptome sequences was obtained from GENCODE site (v32), then processed the cleaned reads with Salmon (1.2.1) (Patro et al., 2017) to get transcript-level expression. Using Tximport (v1.16.0) (Soneson and Robinson, 2018), then summarized to gene-level expression. Finally, the variance stabilizing transformation from DESeq2 (v1.26.0) (Love et al., 2014) was applied to normalize gene expression for downstream analysis and visualization.

### Detection of Viral Reads in RNA-Seq Data

The genome sequences of 501 human viruses were obtained from the NCBI virus database (accessed on Apr. 19, 2020). The list of viral sequences was concatenated with the list of human transcriptome sequences and then a decoy-aware index was built using Salmon. The reads were mapped to the concatenated index using Salmon with a selective-alignment algorithm, which together with the decoy-aware index, mitigates potential spurious mapping of reads arising from unannotated human genomic loci and reduces false positives. Extracted reads were mapped to viral genomes and filtered to remove secondary alignments and paired-end reads with only one mate mapped. The reads were checked with NCBI Nucleotide BLAST to ensure viral origin. The viral read counts were normalized by the total number of sequencing reads of each sample. The correlation between the MVS score and viral read was measured counts using Pearson correlation coefficient.

### Analysis of Single-Cell RNA-Seq Data

The scRNAseq data for (1) the Stanford Cohort from the COVID-19 Cell Atlas (Wilk et al., 2020), and (2) the Atlanta cohort from the NCBI GEO (Arunachalam et al., 2020) was downloaded. scRNAseq data for the Seattle cohort (Su et al., 2020) was processed using Cell Ranger (v3.1.0) (Zheng et al., 2017) and quality control on the three datasets was performed using Seurat (Satija et al., 2015). The read counts were normalized using regularized negative binomial regression with the ‘SCTransform’ function. Then integration workflow was applied in Seurat to integrate the three datasets using canonical correlation analysis. Principal component analysis (PCA), Uniform Manifold Approximation and Projection (UMAP) and Shared Nearest Neighbors clustering was performed on the integrated expression data. Cell type annotation of clusters was performed with both SingleR (Aran et al., 2019) and manual annotation using cell type markers.

In Silico Cellular Deconvolution Using immunoStates and Multi-Cohort Analysis of Estimated Cellular Proportions

In silico cellular deconvolution was done using immunoStates as a basis matrix with support vector regression to estimate proportions of 25 immune cell subsets in each sample (Vallania et al., 2018).

To investigate changes in the immune cell proportions between patients with different severity of viral infection, three multi-cohort analyses were conducted using Metalntegrator R package (Haynes et al., 2017) between samples from the following categories: 1) subjects with non-severe viral infections (severity categories ‘mild’ and ‘moderate’) vs healthy controls, 2) subjects with severe viral infections (severity categories ‘serious’, ‘critical’, and ‘fatal’) vs healthy controls, and 3) subjects with severe viral infections vs subjects with non-severe viral infections. Effect sizes were combined across studies using a random-effects inverse variance model. For each meta-analysis, the change in proportions for each immune cell type between groups in each cohort as the Hedges' g effect size (ES) were calculated. p-values for multiple hypotheses testing were corrected using the Benjamini-Hochberg correction to obtain the false discovery rate (FDR). A threshold of FDR<20% and representation in a minimum of 5 studies in conjunction with leave-one-out analysis was used to identify immune cell types with increased or decreased proportions between groups. Individual samples that met the following criteria were excluded: non-viral infection, non-healthy controls, and one sample from PRJNA252396 (SRR4888654) which had the same expression value for all 317 genes. Datasets with less than two samples in each of the compared groups were excluded from meta-analysis.

### Trajectory Inference Analysis

1674 samples from 21 cohorts in 19 datasets with 1509 samples from four independent challenge studies were co-normalized using COCONUT. Each challenge study inoculated healthy volunteers with one of four viruses (HRV, RSV, H1N1, and H3N2). tSpace, a method for identifying cellular differentiation trajectories using scRNA-seq data (Dermadi et al., 2020), was adapted to identify disease trajectories using bulk transcriptome microarray profiles. The adaption to bulk transcriptome data is referred to as disease space (dSpace) although the core method remains identical to tSpace. The tSpace algorithm involves three steps: (1) calculation of a set of sub-graphs, (2) calculation of the trajectory space matrix across the sub-graphs and (3) visualization. In the first step, a set of sub-graphs keeping L out of K nearest neighbors in a KNN graph were calculated. User defines the number of sub-graphs (G), neighborhood size (K), and how many nearest neighbors will be preserved in the sub-graphs (L). The second step of computes a trajectory space distance matrix using a modified Dijkstra algorithm that implements waypoints (WP) to exponentially weigh and refine distances. The final trajectory space matrix is a dense matrix in which each sample is a row, and calculated trajectories are columns. Number of trajectories (T>150) is user-defined and very robust across wide dynamic range. Finally, we visualize the samples and their relationships in trajectory space using PCA or UMAP.

The following parameters were ised for the dSpace analysis: G=5, K=65, L=49, T=500, WP=20. Pearson correlation was used as the metric for computing distance between two samples. A principal line was fitted through data visualized in the first two components of tSpace (tPC1, tPC2) using the princurve R package. Princurve calculates lambda, an arc length distance for each data point, which we used to align subjects along the isolated trajectory. Furthermore, covariance matrix of the transposed trajectory matrix (covariance mapping) coupled with the hierarchical clustering identified clusters of patients with shared trajectory space. Covariance matrix of the transposed trajectory matrix allows identification of patients that belong to diverging trajectories, and hierarchical clustering of covariance matrix allowed us to group patients that are in severe and non-severe branches, thus enabled isolation of both branches. Each of the determined clusters is a reflection of patients positions in the trajectory space. Hierarchical clustering was calculated using hclust and Dist R functions with “euclidean” and “complete” parameters.

Severe and non-severe branches shared a substantial number of healthy patients. Therefore, they were aligned using dynamic time warping (dtw R package) and split them into 4 stages. All 251 genes and the fitted trajectory (lambda value) were used for alignment. A permutation test (Efron and Tibshirani, 2002) was applied for each of the 4 stages and identified total of 96 genes that were differentially expressed within the same stage between the two severity branches. In our testing we used 1000 permutations, and for significance FDR<0.001 and |effects size|>0.3.

### Calculation of the SoM Score

The Severe or Mild (SoM) score can calculated using a 42-gene model that utilizes the expression of genes from the 4 gene modules to distinguish between severe and mild viral infections. Equivalent results can be used if less genes are used. For each sample, the geometric mean of the expression of genes from each module. Then, we calculate a score by taking the sum of the geometric means of modules 1 and 2 and dividing that by the sum of the geometric means of modules 3 and 4, as shown in the following equation:

$$SoM score = \frac{\begin{matrix}{( {\prod }_{gene ∈ Module 1} {x}_{i} ( gene ) )}^{\frac{1}{‖ Module 1 ‖}} + \\ {( {\prod }_{gene ∈ Module 2} {x}_{i} ( gene ) )}^{\frac{1}{‖ Module 2 ‖}}\end{matrix}}{\begin{matrix}{( {\prod }_{gene ∈ Module 3} {x}_{i} ( gene ) )}^{\frac{1}{‖ Module 3 ‖}} + \\ {( {\prod }_{gene ∈ Module 4} {x}_{i} ( gene ) )}^{\frac{1}{‖ Module 4 ‖}}\end{matrix}} . \qquad ( 1 )$$

SoM Score from Nasal Swab Samples Correlates with Severity of Viral Infection

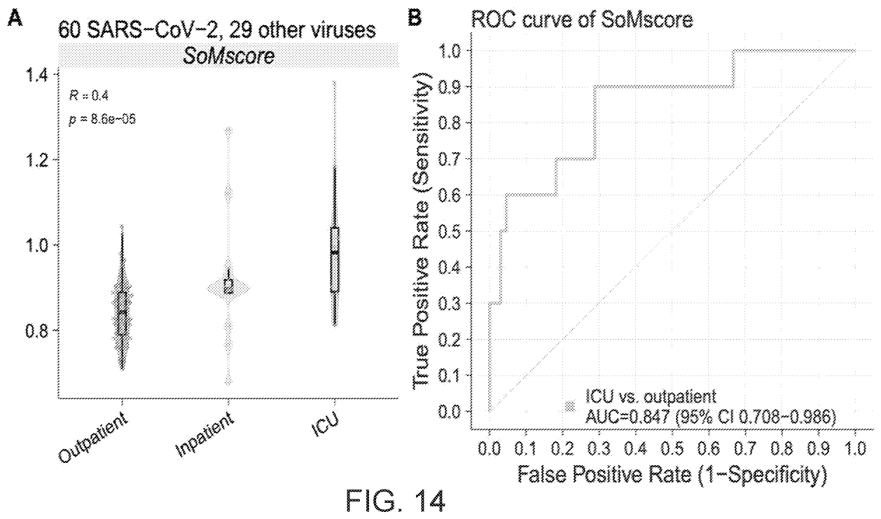

Gene expression data from in nasal swab samples from patients with SARS-CoV-2 (N=60) and other viral infections (N=29) 1. Patients were classified into three groups according to disease severity: outpatient, inpatient, and ICU. SoM score, calculated from four gene modules, was correlated with disease severity (R=0.4, p=8.6e-05) (FIG. 14A). The AUROC of SoM score in differentiating ICU patients from outpatients is 0.847 (95% Cl 0.708-0.986) (FIG. 14B). Collectively, the biomarkers for predicting severe outcome in patients with infection can be measured not only in blood, but also in nasal swab samples.

### REFERENCES

Andres-Terre, M., McGuire, H. M., Pouliot, Y., Bongen, E., Sweeney, T. E., Tato, C. M., and Khatri, P. (2015). Integrated, Multi-cohort Analysis Identifies Conserved Transcriptional Signatures across Multiple Respiratory Viruses. Immunity 43, 1199-1211.
Aran, D., Looney, A. P., Liu, L., Wu, E., Fong, V., Hsu, A., Chak, S., Naikawadi, R. P., Wolters, P. J., Abate, A. R., et al. (2019). Reference-based analysis of lung single-cell sequencing reveals a transitional profibrotic macrophage. Nature Immunology 20, 1-15.
Arunachalam, P. S., Wimmers, F., Mok, C. K. P., Perera, R. A. P. M., Scott, M., Hagan, T., Sigal, N., Feng, Y., Bristow, L., Tak-Yin Tsang, O., et al. (2020). Systems biological assessment of immunity to mild versus severe COVID-19 infection in humans. Science 369, 1210-1220.
Bailey, C. C., Zhong, G., Huang, I.-C., and Farzan, M. (2014). IFITM-Family Proteins: The Cell's First Line of Antiviral Defense. Annu Rev Virol 1, 261-283.
Bertini, R., Howard, O. M., Dong, H. F., Oppenheim, J. J., Bizzarri, C., Sergi, R., Caselli, G., Pagliei, S., Romines, B., Wilshire, J. A., et al. (1999). Thioredoxin, a redox enzyme released in infection and inflammation, is a unique chemoattractant for neutrophils, monocytes, and T cells. The Journal of Experimental Medicine 189, 1783-1789.
Bongen, E., Vallania, F., Utz, P. J., and Khatri, P. (2018). KLRD1-expressing natural killer cells predict influenza susceptibility. Genome Med 10, 1-12.
Bruns, H., Büttner, M., Fabri, M., Mougiakakos, D., Bittenbring, J. T., Hoffmann, M. H., Beier, F., Pasemann, S., Jitschin, R., Hofmann, A. D., et al. (2015). Vitamin D-dependent induction of cathelicidin in human macrophages results in cytotoxicity against high-grade B cell lymphoma. Science Translational Medicine 7, 282ra47-282ra47.
Choi, J. W., and Fujii, T. (2019). The Prevalence of Low Plasma Neutrophil Gelatinase-Associated Lipocalin Level in Systemic Inflammation and its Relationship with Proinflammatory Cytokines, Procalcitonin, Nutritional Status, and Leukocyte Profiles. Clin. Lab. 65.
Chowdhury, R. R., Vallania, F., Yang, Q., Angel, C. J. L., Darboe, F., Penn-Nicholson, A., Rozot, V., Nemes, E., Malherbe, S. T., Ronacher, K., et al. (2018). A multi-cohort study of the immune factors associated with M. tuberculosis infection outcomes. Nature 560, 1-23.
Christiansen, J. (2018). Global Infections by the Numbers. Scientific American 318, 48-49.
David M Morens, M. D., and Anthony S Fauci, M. D. (2020). Emerging Pandemic Diseases: How We Got To COVID-19. Cell Research 1-76.
Dermadi, D., Bscheider, M., Bjegovic, K., Lazarus, N. H., Szade, A., Hadeiba, H., and Butcher, E. C. (2020). Exploration of Cell Development Pathways through High-Dimensional Single Cell Analysis in Trajectory Space. Iscience 23, 100842.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Efron, B., and Tibshirani, R. (2002). Empirical bayes methods and false discovery rates for microarrays. Genetic Epidemiology 23, 70-86.
Eriksson, J., Gidlöf, A., Eriksson, M., Larsson, E., Brattström, O., and Oldner, A. (2017). Thioredoxin a novel biomarker of post-injury sepsis. Free Radic. Biol. Med. 104, 138-143.
Facão, A. L. E., Barros, A. G. de A., Bezerra, A. A. M., Ferreira, N. L., Logato, C. M., Silva, F. P.,
do Monte, A. B. F. O., Tonella, R. M., de Figueiredo, L. C., Moreno, R., et al. (2019). The prognostic accuracy evaluation of SAPS 3, SOFA and APACHE II scores for mortality prediction in the surgical ICU: an external validation study and decision-making analysis. Ann Intensive Care 9, 18-10.
García-Alcalde, F., Okonechnikov, K., Carbonell, J., Cruz, L. M., Götz, S., Tarazona, S., Dopazo, J., Meyer, T. F., and Conesa, A. (2012). Qualimap: evaluating next-generation sequencing alignment data. Bioinformatics 28, 2678-2679.
Gatti, A., Radrizzani, D., Viganó, P., Mazzone, A., and Brando, B. (2020). Decrease of Non-Classical and Intermediate Monocyte Subsets in Severe Acute SARS-CoV-2 Infection. Cytometry A 30, 371.
Gupta, R. K., Turner, C. T., Venturini, C., Esmail, H., Rangaka, M. X., Copas, A., Lipman, M., Abubakar, I., and Noursadeghi, M. (2020). Concise whole blood transcriptional signatures for incipient tuberculosis: a systematic review and patient-level pooled meta-analysis. The Lancet Resp Med 8, 395-406.
Hadjadj, J., Yatim, N., Barnabei, L., Corneau, A., Boussier, J., Smith, N., Pere, H., Charbit, B., Bondet, V., Chenevier-Gobeaux, C., et al. (2020). Impaired type I interferon activity and inflammatory responses in severe COVID-19 patients. Science 369, 718-724.
Haynes, W. A., Vallania, F., Liu, C., Bongen, E., Tomczak, A., Andres-Terre, M., Lofgren, S., Tam, A., Deisseroth, C. A., Li, M. D., et al. (2017). EMPOWERING MULTI-COHORT GENE EXPRESSION ANALYSIS TO INCREASE REPRODUCIBILITY. Pac Symp Biocomput 22, 144-153.
Johnson, W. E., Li, C., and Rabinovic, A. (2006). Adjusting batch effects in microarray expression data using empirical Bayes methods. Biostatistics 8, 118-127.
Liao, Y., Goraya, M. U., Yuan, X., Zhang, B., Chiu, S.-H., and Chen, J.-L. (2019). Functional Involvement of Interferon-Inducible Transmembrane Proteins in Antiviral Immunity. Front Microbiol 10, 1097.
Liu, F., Li, L., Xu, M., Wu, J., Luo, D., Zhu, Y., Li, B., Song, X., and Zhou, X. (2020). Prognostic value of interleukin-6, C-reactive protein, and procalcitonin in patients with COVID-19. J. Clin. Virol. 127, 104370.
Love, M. I., Huber, W., and Anders, S. (2014). Moderated estimation of fold change and dispersion for RNA-seq data with DESeq2. Genome Biol. 15, 550-21.
Mace, E. M. (2018). Phosphoinositide-3-Kinase Signaling in Human Natural Killer Cells: New Insights from Primary Immunodeficiency. Front Immunol 9, 310-311.
Mayhew, M. B., Buturovic, L., Luethy, R., Midic, U., Moore, A. R., Roque, J. A., Shaller, B. D., Asuni, T., Rawling, D., Remmel, M., et al. (2020). A generalizable 29-mRNA neural-network classifier for acute bacterial and viral infections. Nat Commun 1-10.
Nakamura, K., Ito, I., Kobayashi, M., Herndon, D. N., and Suzuki, F. (2015). Orosomucoid 1 drives opportunistic infections through the polarization of monocytes to the M2b phenotype. Cytokine 73, 8-15.
Papathanassiu, A. E., Ko, J.-H., Imprialou, M., Bagnati, M., Srivastava, P. K., Vu, H. A., Cucchi, D., McAdoo, S. P., Ananieva, E. A., Mauro, C., et al. (2017). BCAT1 controls metabolic reprogramming in activated human macrophages and is associated with inflammatory diseases. Nat Commun 8, 16040-13.
Patro, R., Duggal, G., Love, M. I., Irizarry, R. A., and Kingsford, C. (2017). Salmon provides fast and bias-aware quantification of transcript expression. Nature Publishing Group 14, 417-419.
Ramos-Martínez, E., López-Vancell, M. R., de Córdova-Aguirre, J. C. F., Rojas-Serrano, J., Chavarría, A., Velasco-Medina, A., and Veldzquez-Sámano, G. (2018). Reduction of respiratory infections in asthma patients supplemented with vitamin D is related to increased serum IL-10 and IFNy levels and cathelicidin expression. Cytokine 108, 239-246.
Rast, A. C., Mueller, B., and Schuetz, P. (2014). Clinical scores and blood biomarkers for early risk assessment of patients presenting to the emergency department. OA Emergency Medicine 2,1-9.
Satija, R., Farrell, J. A., Gennert, D., Schier, A. F., and Regev, A. (2015). Spatial reconstruction of single-cell gene expression data. Nature Biotechnology 33, 495-502.
Schrijver, I. T., Théroude, C., and Roger, T. (2019). Myeloid-Derived Suppressor Cells in Sepsis. Front Immunol 10, 327.
Scott, M. K. D., Quinn, K., Li, Q., Carroll, R., Warsinske, H., Vallania, F., Chen, S., Carns, M. A., Aren, K., Sun, J., et al. (2019). Increased monocyte count as a cellular biomarker for poor outcomes in fibrotic diseases: a retrospective, multicentre cohort study. The Lancet Resp Med 1-12.
Silvin, A., Chapuis, N., Dunsmore, G., Goubet, A.-G., Dubuisson, A., Derosa, L., Almire, C., Hénon, C., Kosmider, O., Droin, N., et al. (2020). Elevated calprotectin and abnormal myeloid cell subsets discriminate severe from mild COVID-19. Cell Research 1-45.
Soneson, C., and Robinson, M. D. (2018). Bias, robustness and scalability in single-cell differential expression analysis. Nature Publishing Group 1-15.
Su, Y., Chen, D., Lausted, C., Yuan, D., Choi, J., Dai, C., Voillet, V., Scherler, K., Troisch, P., Duvvuri, V. R., et al. (2020). Multiomic Immunophenotyping of COVID-19 Patients Reveals Early Infection Trajectories. bioRxiv 2020.07.27.224063.
Sweeney, T. E., Braviak, L., Tato, C. M., and Khatri, P. (2016a). Genome-wide expression for diagnosis of pulmonary tuberculosis: a multicohort analysis. The Lancet Resp Med 4, 213-224.
Sweeney, T. E., Perumal, T. M., Henao, R., Nichols, M., Howrylak, J. A., Choi, A. M., Bermejo-Martin, J. F., Almansa, R., Tamayo, E., Davenport, E. E., et al. (2018a). A community approach to mortality prediction in sepsis via gene expression analysis. Nat Commun 9, 1-10.
Sweeney, T. E., Perumal, T. M., Henao, R., Nichols, M., Howrylak, J. A., Choi, A. M., Bermejo-Martin, J. F., Almansa, R., Tamayo, E., Davenport, E. E., et al. (2018b). A community approach to mortality prediction in sepsis via gene expression analysis. Nat Commun 9, 694-10.
Sweeney, T. E., Shidham, A., Wong, H. R., and Khatri, P. (2015). A comprehensive time-course-based multicohort analysis of sepsis and sterile inflammation reveals a robust diagnostic gene set. Science Translational Medicine 7, 287ra71-287ra71.
Sweeney, T. E., Wong, H. R., and Khatri, P. (2016b). Robust classification of bacterial and viral infections via integrated host gene expression diagnostics. Science Translational Medicine 8, 346ra91-346ra91.
Takizawa, H., Boettcher, S., and Manz, M. G. (2012). Demand-adapted regulation of early hematopoiesis in infection and inflammation. Blood 119, 2991-3002.
Turner, C. T., Gupta, R. K., Tsaliki, E., Roe, J. K., Mondal, P., Nyawo, G. R., Palmer, Z., Miller, R. F., Reeve, B. W., Theron, G., et al. (2020). Blood transcriptional biomarkers for active pulmonary tuberculosis in a high-burden setting: a prospective, observational, diagnostic accuracy study. The Lancet Resp Med 8, 407-419.
Vallania, F., Tam, A., Lofgren, S., Schaffert, S., Azad, T. D., Bongen, E., Haynes, W., Alsup, M., Alonso, M., Davis, M., et al. (2018). Leveraging heterogeneity across multiple datasets increases cell-mixture deconvolution accuracy and reduces biological and technical biases. Nat Commun 9,1-8.
Warsinske, H. C., Rao, A. M., Moreira, F. M. F., Santos, P. C. P., Liu, A. B., Scott, M., Malherbe,
S. T., Ronacher, K., WaIzI, G., Winter, J., et al. (2018). Assessment of Validity of a Blood-Based 3-Gene Signature Score for Progression and Diagnosis of Tuberculosis, Disease Severity, and Treatment Response. JAMA Network Open 1, e183779-13.
Wei, F., Jiang, Z., Sun, H., Pu, J., Sun, Y., Wang, M., Tong, Q., Bi, Y., Ma, X., Gao, G. F., et al. (2019). Induction of PGRN by influenza virus inhibits the antiviral immune responses through downregulation of type I interferons signaling. PLoS Pathog 15, e1008062.
Wilk, A. J., Rustagi, A., Zhao, N. Q., Roque, J., Martínez-Colón, G. J., McKechnie, J. L., Ivison, G. T., Ranganath, T., Vergara, R., Hollis, T., et al. (2020). A single-cell atlas of the peripheral immune response in patients with severe COVID-19. Nat Med 1-21.
Wu, T., Ji, Y., Moseman, E. A., Xu, H. C., Manglani, M., Kirby, M., Anderson, S. M., Handon, R., Kenyon, E., Elkahloun, A., et al. (2016). The TCF1-Bcl6 axis counteracts type I interferon to repress exhaustion and maintain T cell stemness. Science Immunology 1, eaai8593-eaai8593.
Wu, Z., and McGoogan, J. M. (2020). Characteristics of and Important Lessons From the Coronavirus Disease 2019 (COVID-19) Outbreak in China: Summary of a Report of 72314 Cases From the Chinese Center for Disease Control and Prevention. JAMA: the Journal of the American Medical Association 323, 1239-1242.
Zhao, X., Li, J., Winkler, C. A., An, P., and Guo, J.-T. (2018). IFITM Genes, Variants, and Their Roles in the Control and Pathogenesis of Viral Infections. Front Microbiol 9, 3228.
Zheng, G. X. Y., Terry, J. M., Belgrader, P., Ryvkin, P., Bent, Z. W., Wilson, R., Ziraldo, S. B., Wheeler, T. D., McDermott, G. P., Zhu, J., et al. (2017). Massively parallel digital transcriptional profiling of single cells. Nat Commun 8, 14049-12.
Zhou, Z., Ren, L., Zhang, L., Zhong, J., Xiao, Y., Jia, Z., Guo, L., Yang, J., Wang, C., Jiang,
S., et al. (2020). Heightened Innate Immune Responses in the Respiratory Tract of COVID-19 Patients. Cell Host Microbe 27, 883-890.e2.

1. A method for determining a virally-infected subject's risk of developing of severe symptoms, comprising:
(a) measuring the amount of RNA transcripts encoded by at least two of HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFITM1, DDB1, BCL2L11, LAPTM4A, KIF23, TYK2, PIK3R1, BANF1, TRIM28, SOCS6, LRBA, ANXA2, IFITM3, CREG1, and NAPA in a sample of RNA obtained from the subject, to obtain gene expression data; and
(b) based on the gene expression data, providing a report indicating the subject's risk of developing severe symptoms, wherein:
(i) increased expression of BCL6, NQO2, ORM1, DEFA4, CTSG, LCN2, AZU1, TXN, CCL2, CEACAM8, AQP9, GRN, CAMP, TLR2, ANXA3, SLPI, KLHL2, CEP55, SRGN, TRIP13, PRC1, TCEAL9, BCAT1, FURIN, ACSL1, HMMR, UBE2L6, CASP7, OLR1, SCAND1, DOK3, KIF15, ATP8B4, IGFBP2, IFITM2, PFKFB4, VAMP5, ELL2, POMP, H1-0, ADM, VRK2, MAFB, CDT1, OASL, TMEM123, TLN1, NUCB1, FAM8A1, BTBD7, ATG3, BCL2A1, IFITM1, BCL2L11, KIF23, SOCS6, ANXA2, IFITM3, CREG1 and NAPA; and
(ii) decreased expression of HLA-DPB1, KLRB1, DOK2, KLRG1, KLRD1, EPHX2, EXOC2, PRF1, PRSS23, TRIB2, EZH1, BUB3, ITGB7, SIDT1, RAD23B, ARHGAP45, MAP3K4, USP11, SMYD2, SSR2, IL7R, FBLN5, TRAF5, TRAF31P3, CCR7, LTBP3, CHMP7, PITPNC1, RBM15B, DDB1, LAPTM4A, TYK2, PIK3R1, BANF1, TRIM28 and LRBA
increases the risk of the subject will develop severe symptoms.

2. The method of claim 1, wherein the measuring step is done by sequencing.

3. The method of claim 1, wherein the measuring step is done by RT-PCR or an isothermal quantification method.

4. The method of claim 1, wherein the measuring step is done by labeling the RNA or cDNA made from the same and hybridizing the labeled RNA or cDNA to a support.

5. The method of claim 1, wherein the RNA is isolated from a nasal swab, whole blood, peripheral blood mononuclear cells, white blood cells, neutrophils or buffy coat.

6. The method of claim 1, wherein step (b) comprises calculating a severe or mile (SoM) score based on the amounts of the RNA transcripts, wherein the score indicates the probability that the subject will develop severe symptoms.

7. The method of claim 1, wherein the RNA transcripts analyzed in step (a) comprise at least one gene from each of the following modules:
module 1: NQO2, SLPI, ORM1, KLHL2, ANXA3, TXN, AQP9, BCL6, DOK3, PFKFB4 and TYK2;
module 2: BCL2L11, BCAT1, BTBD7, CEP55, HMMR, PRC1, KIF15, CAMP, CEACAM 8, DEFA4, LCN2, CTSG and AZU1;
module 3: MAFB, OASL, UBE2L6, VAMP5, CCL2, NAPA, ATG3, VRK2, TMEM123 and CASP7; and
module 4: DOK2, HLA-DPB1, BUB3, SMYD2, SIDT1, EXOC2, TRIB2 and KLRB1.

8. A method for treating a subject having a viral infection, comprising:
(c) receiving a report indicating a virally-infected subject's risk of developing severe symptoms, wherein the report is based on the gene expression data obtained by measuring the amount of RNA transcripts encoded by at least two of HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFITM1, DDB1, BCL2L11, LAPTM4A, KIF23, TYK2, PIK3R1, BANF1, TRIM28, SOCS6, LRBA, ANXA2, IFITM3, CREG1, and NAPA in a sample of RNA obtained from the subject, wherein:
(i) increased expression of BCL6, NQO2, ORM1, DEFA4, CTSG, LCN2, AZU1, TXN, CCL2, CEACAM8, AQP9, GRN, CAMP, TLR2, ANXA3, SLPI, KLHL2, CEP55, SRGN, TRIP13, PRC1, TCEAL9, BCAT1, FURIN, ACSL1, HMMR, UBE2L6, CASP7, OLR1, SCAND1, DOK3, KIF15, ATP8B4, IGFBP2, IFITM2, PFKFB4, VAMP5, ELL2, POMP, H1-0, ADM, VRK2, MAFB, CDT1, OASL, TMEM123, TLN1, NUCB1, FAM8A1, BTBD7, ATG3, BCL2A1, IFITM1, BCL2L11, KIF23, SOCS6, ANXA2, IFITM3, CREG1 and NAPA; and
(ii) decreased expression of HLA-DPB1, KLRB1, DOK2, KLRG1, KLRD1, EPHX2, EXOC2, PRF1, PRSS23, TRIB2, EZH1, BUB3, ITGB7, SIDT1, RAD23B, ARHGAP45, MAP3K4, USP11, SMYD2, SSR2, IL7R, FBLN5, TRAF5, TRAF31P3, CCR7, LTBP3, CHMP7, PITPNC1, RBM15B, DDB1, LAPTM4A, TYK2, PIK3R1, BANF1, TRIM28 and LRBA
increases the subject's risk of developing severe symptoms; and
(b) treating the subject based on whether the subject has a high risk of developing severe symptoms.

9. The method of claim 8, wherein the comparing the risk to a threshold, determining that the risk is above a threshold, and administering intensive care or an antiviral therapy to the patient.

10. The method of claim 9, wherein the intensive care comprises one or more of providing supplemental oxygen to the patient, putting the patient on mechanical ventilation, connecting the patient with a device to monitor a bodily function selected from one or more of heart and pulse rate, air flow to the lungs, blood pressure, blood flow, central venous pressure, amount of oxygen in the blood, and body temperature, and adding an intravenous line to the patient.

11. The method of claim 9, wherein the antiviral therapy comprises administering a therapeutic dose of camostat mesylate, nafamostat mesylate, chloroquine phosphate, hydroxychloroquine, cepharanthine/selamectin/mefloquine hydrochloride, remdesivir, N4, hydroxyctidine, lopinavir/ritonavir, umifenovir, favipiravir, oseltamivir or N3 to the subject.

12. The method of claim 9, wherein the antiviral therapy comprises administering a therapeutic dose of broad-spectrum antiviral agent, an antiviral vaccine, a neuraminidase inhibitor (e.g., zanamivir (Relenza) and oseltamivir (Tamiflu)), a nucleoside analogue (e.g., acyclovir, zidovudine (AZT), and lamivudine), an antisense antiviral agent (e.g., phosphorothioate antisense antiviral agents (e.g., Fomivirsen (Vitravene) for cytomegalovirus retinitis), morpholino antisense antiviral agents), an inhibitor of viral uncoating (e.g., Amantadine and rimantadine for influenza, Pleconaril for rhinoviruses), an inhibitor of viral entry (e.g., Fuzeon for HIV), an inhibitor of viral assembly (e.g., Rifampicin), or an antiviral agent that stimulates the immune system (e.g., interferons). Exemplary antiviral agents include Abacavir, Aciclovir, Acyclovir, Adefovir, Amantadine, Amprenavir, Ampligen, Arbidol, Atazanavir, Atripla (fixed dose drug), Balavir, Cidofovir, Combivir (fixed dose drug), Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type Ill, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), or Zidovudine to the patient.

13-14. (canceled)

15. A kit comprising reagents for measuring the amount of RNA transcripts encoded by at least 2, at least 3, at least 5, at least 10, at least 15, at least 20, at least 30, at least 40 or at least 50 or all of HLA-DPB1, BCL6, NQO2, ORM1, DEFA4, KLRB1, CTSG, LCN2, AZU1, TXN, DOK2, CCL2, CEACAM8, AQP9, KLRG1, KLRD1, EPHX2, GRN, CAMP, TLR2, ANXA3, SLPI, KLHL2, CEP55, SRGN, TRIP13, PRC1, TCEAL9, EXOC2, BCAT1, PRF1, PRSS23, TRIB2, FURIN, ACSL1, EZH1, HMMR, UBE2L6, CASP7, OLR1, BUB3, SCAND1, ITGB7, DOK3, SIDT1, RAD23B, KIF15, ARHGAP45, MAP3K4, ATP8B4, IGFBP2, IFITM2, USP11, SMYD2, PFKFB4, VAMP5, ELL2, POMP, H1-0, ADM, SSR2, VRK2, IL7R, FBLN5, MAFB, TRAF5, CDT1, OASL, TRAF31P3, TMEM123, TLN1, CCR7, LTBP3, CHMP7, PITPNC1, NUCB1, RBM15B, FAM8A1, BTBD7, ATG3, BCL2A1, IFITM1, DDB1, BCL2L11, LAPTM4A, KIF23, TYK2, PIK3R1, BANF1, TRIM28, SOCS6, LRBA, ANXA2, IFITM3, CREG1, and NAPA.

16. The kit of claim 15, wherein the reagents comprise, for each RNA transcript, a sequence-specific oligonucleotide that hybridizes to the transcript.

17. The kit of claim 16, wherein sequence-specific oligonucleotide is biotinylated and/or labeled with an optically-detectable moiety.

18. The kit of claim 15, wherein the reagents comprise, for each RNA transcript, a pair of PCR primers that amplify a sequence from the RNA transcript, or cDNA made from the same.

19. The kit of claim 15, wherein the reagents comprise an array of oligonucleotide probes, wherein the array comprises, for each RNA transcript, at least one sequence-specific oligonucleotide that hybridizes to the transcript.
